(19) United States
(12) Patent Application Publication
(10) Pub. No.: US 2025/0092171 A1
(43) Pub. Date: Mar. 20, 2025

(54) HIGH-PURITY 4-(2-BROMOETHYL)BENZENESULFONIC ACID, HIGH-PURITY STYRENESULFONIC ACID COMPOUND DERIVED THEREFROM, AND POLYMER THEREOF, AND METHODS FOR PRODUCING SAME

(71) Applicant: TOSOH FINECHEM CORPORATION, Shunan-shi (JP)

(72) Inventors: Shinji OZOE, Shunan-shi (JP); Yusuke SHIGETA, Shunan-shi (JP); Hiroshi AWANO, Shunan-shi (JP)

(73) Assignee: TOSOH FINECHEM CORPORATION, Shunan-shi (JP)

(21) Appl. No.: 18/828,269

(22) Filed: Sep. 9, 2024

Related U.S. Application Data
(63) Continuation of application No. PCT/JP2023/008234, filed on Mar. 6, 2023.

(30) Foreign Application Priority Data
Mar. 9, 2022 (JP) 2022-035847

(51) Int. Cl. C08F 112/08; C07C 303/06; C07C 311/15

(52) U.S. Cl. CPC C08F 112/08; C07C 303/06; C07C 311/15

(57) ABSTRACT

Provided are a high-purity styrenesulfonic acid compound having a markedly decreased amount of bonded bromine and a polymer thereof, which are useful for modifiers for secondary batteries, dopants for conductive polymers, additives for semiconductor polishing and cleaning agents, organic EL elements, and photoresists, and particularly for members for electronic materials. A high-purity 4-(2-bromoethyl)benzenesulfonic acid having a decreased amount of nuclear-brominated forms, a high-purity styrenesulfonic acid compound having a markedly decreased amount of bonded bromine, which are derived from the high-purity 4-(2-bromoethyl)benzenesulfonic acid, and a polymer of the high-purity styrenesulfonic acid compound are used.

### TECHNICAL FIELD

The present invention relates to a high-purity 4-(2-bromoethyl)benzenesulfonic acid having a decreased amount of nuclear-brominated forms, a high-purity styrenesulfonic acid compound derived therefrom and having a decreased amount of bonded bromine, and a polymer thereof, and methods for producing the same.

### BACKGROUND ART

Styrenesulfonic acid compounds and polystyrenesulfonic acid compounds derived therefrom are functional monomers and polymers thereof, which are used for fuel cell films, polymeric solid electrolytes and additives for secondary batteries, dispersants also serving as dopants for conductive polymers and carbon nanotubes, semiconductor cleaning agents, electron-accepting substances and thermal acid generators for organic EL elements, thermal acid generators and protective films for photoresists, and the like (see, for example, Patent Documents 1 to 5). Since use in the field of electronic materials is dominant, high-purity styrenesulfonic acid compounds and polymers thereof are required in which impurities such as halogen that causes metal corrosion are reduced as much as possible (see, for example, Patent Document 6).

Among the styrenesulfonic acid compounds, styrenesulfonic acid esters are particularly useful in the above-described applications because they are oil-soluble liquid monomers, and are easily copolymerized with an oil-soluble monomers or subjected to a coating process to form a polymer coating film on various substrate surfaces or produce a polymer electrolyte film. In addition, styrenesulfonic acid salts such as styrenesulfonic acid amine salts and lithium salts are useful in that they have high solubility in water and aprotic polar solvents, thus can be applied to a coating process, and enable a polystyrenesulfonic acid aqueous solution to be produced without an organic solvent (see, for example, Patent Document 7).

A styrenesulfonic acid ester can be produced by the following method (e.g., Non-Patent Document 1). That is, a styrenesulfonic acid salt such as sodium styrenesulfonate and thionyl chloride are reacted to obtain styrenesulfonyl chloride, which is then esterified using a base such as potassium hydroxide and alcohol.

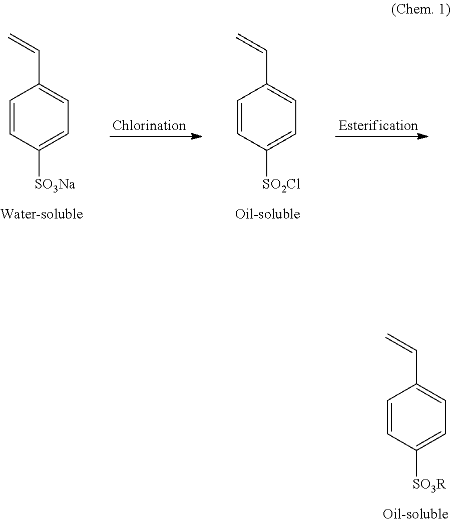

Applications of polymerizable styrenesulfonic acid esters which are vinyl monomers include production of aqueous colloids of a conductive polymer with a polystyrenesulfonic acid whose molecular weight distribution is controlled (e.g., Patent Document 2). For example, it has been reported that a polystyrenesulfonic acid aqueous solution can be produced by performing living radical polymerization of ethyl styrenesulfonate, then hydrolyzing ester groups with sodium hydroxide, and subsequently removing metal cations, low-molecular impurities and unreacted monomers using a cation-exchange resin and an ultrafiltration membrane.

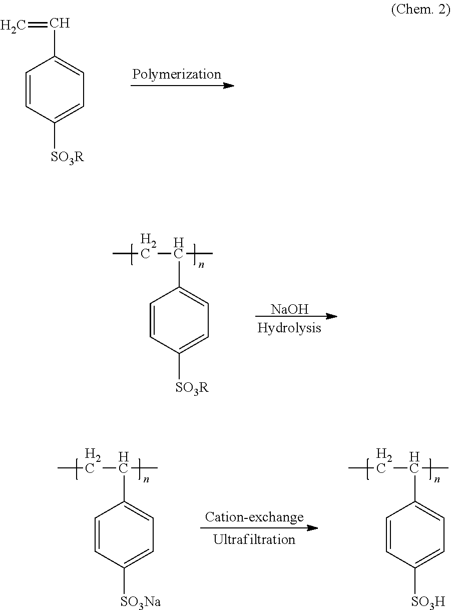

The polystyrenesulfonic acid can be produced by another method. For example, it has been reported that a polystyrenesulfonic acid aqueous solution can be produced by performing radical polymerization of sodium styrenesulfonate in water, adding sodium hydroxide, heating the mixture at 60° C., and then performing purification using a cation-exchange resin and an ultrafiltration membrane as described above (e.g., Patent Document 7).

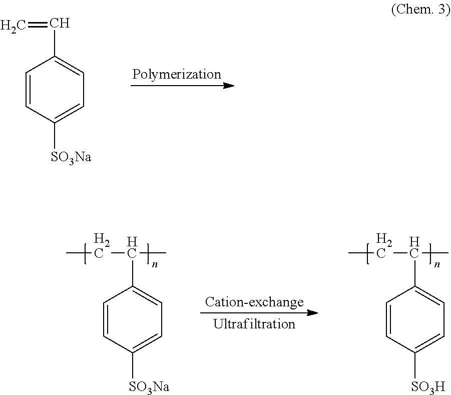

The polystyrenesulfonic acid can be produced by still another method. That is, polystyrene is sulfonated in a solvent inactive against a sulfonating agent (e.g., Patent Document 8). The production method has an advantage that ingress of halogenated alkali metal hardly occurs, as well as a disadvantage that the degree of freedom in the polymer design related to, for example, the polymer composition and molecular weight is low, and branched structures are easily formed.

### PRIOR ART DOCUMENT

### Patent Document

Patent Document 1: Japanese Patent Laid-Open No. 2017-43700
Patent Document 2: Japanese Patent Laid-Open No. 2011-213823
Patent Document 3: International Publication No. WO 2015/114966
Patent Document 4: International Publication No. WO 2007/099808
Patent Document 5: Japanese Patent Laid-Open No. 2004-177666
Patent Document 6: Japanese Patent Laid-Open No. 1993-326477
Patent Document 7: Japanese Patent Laid-Open No. 2009-1624
Patent Document 8: Japanese Patent Laid-Open No. 1996-299777

### Non-Patent Document

Non-Patent Document 1: H. Okamura et al., Polymer, Vol 43, pp. 3155-3162, 2002

### SUMMARY OF THE INVENTION

### Problems to be Solved by the Invention

As described above, heretofore, high-purity styrenesulfonic acid compounds and polymers thereof in which halogen impurities are reduced as much as possible have been required, and it has been necessary to reduce halogen impurities.

Patent Document 7 discloses a polystyrenesulfonic acid which has a number average molecular weight of 50,000 to 1,000,000 and in which the total amount of remaining bromine and chlorine is 500 ppm (on a mass basis) or less, and the amount of remaining styrenesulfonic acid monomers is 1 mass % or less. Sodium styrenesulfonate is polymerized, the resulting sodium polystyrenesulfonate is then treated with an alkali such as ammonia or sodium hydroxide to release bromine and chlorine, which are removed by an ethanol precipitation method or an ultrafiltration method, and a sodium polystyrenesulfonate aqueous solution free of bromine, chlorine and unreacted sodium styrenesulfonate is obtained (claim 1 and paragraph [0030]). However, bromine sources and chlorine sources are not specifically described, and are unknown.

The ion-chromatographic analysis of the halogen concentration in an aqueous solution of a polystyrenesulfonic acid prepared in the same manner as described above indeed shows that for example, a bromide ion concentration of less than 1 ppm can be apparently achieved in the fresh aqueous solution immediately after the production. However, the result of detailed examination of the long-term stability of the polystyrenesulfonic acid aqueous solution by the present inventors has revealed that the bromide ion concentration in the aqueous solution markedly increases with time even under mild conditions, indicating that there remains a problem. That is, the presence of previously unreported labile bonded bromine which cannot be removed by the above-described purification method has been pointed out. It has been heretofore difficult to identify a cause of increasing the bromide ion concentration with time and obtain a polystyrenesulfonic acid that can be preserved for a long period.

For the bonded bromine in sodium styrenesulfonate, the presence of sodium bromostyrenesulfonate has been reported (e.g., International Publication No. WO 2014/061357). The patent discloses high-purity sodium styrenesulfonate having a sodium bromostyrenesulfonate content of 0.01% (on a basis of an area measured by high-performance liquid chromatography) (paragraph in the publication of the patent).

Specifically, Example 2 in the publication of the patent shows high-purity sodium para-styrenesulfonate, production of PSS sodium, and Evaluation Example 2 as synthetic glue for a fabric conditioner for use in ironing of clothes, which will be described below. Note that in relation to the following area-based relative values of the peak areas of compounds (a) to (e) which are measured by high-performance liquid chromatography, (a) represents sodium ortho-styrenesulfonate, (b) represents sodium β-bromoethylbenzenesulfonate, (c) represents sodium meta-styrenesulfonate, (d) represents sodium bromostyrenesulfonate, and (e) represents sodium β-hydroxyethylbenzenesulfonate.

<Production of High-Purity Sodium Para-Styrenesulfonate>

A jacketed stainless reactor equipped with a stirrer was charged with 1,000 g of the high-purity sodium para-styrenesulfonate obtained in Example 1, 1 g of sodium nitrite, 20 g of sodium hydroxide and 950 g of pure water, and the mixture was stirred in a nitrogen atmosphere at 60° C. for 1 hour. Thereafter, the reaction liquid was cooled to room temperature over 3 hours, and then subjected to solid-liquid separation with a centrifuge to obtain 899 g of a wet cake of high-purity sodium para-styrenesulfonate.

The high-purity sodium para-styrenesulfonate had a purity of 89.1 wt %, and contained a moisture content at 8.2 wt %, an iron content at 0.58 μg/g, a sodium bromide content at 0.20 wt %, and organic impurities such as isomers at 0.05% for (a), 0.00% for (b), 1.34% for (c), 0.01% for (d) and 0.01% for (e). The sodium para-styrenesulfonate had a median diameter of 63 μm, contained small grains of less than 10.00 μm at 2.0%, and had a repose angle of 49 degrees, and it took 155 seconds for the sodium para-styrenesulfonate to dissolve in water. The sodium para-styrenesulfonate had a WI value of 96.5, a YI value of 2.9, and an APHA value of 15 in the form of a 15 wt % aqueous solution, and was obviously superior in hue to conventional products (Comparative Example 1). Further, for unknown reasons, it is obvious that reduction of impurities such as sodium bromide and isomers further improves the hue although the iron content level is equivalent to that in Example 1.

In the patent, the purpose to be achieved is to improve the hue of sodium para-styrenesulfonate and PSS sodium (sodium polystyrenesulfonate), and the hue is improved with the synergic effect of simultaneously reducing an iron content and various organic impurities that may be contained in the sodium para-styrenesulfonate. As a specific method, an iron content in a 4-(2-bromoethyl)benzenesulfonic acid aqueous solution which is a precursor of sodium para-styrenesulfonate is eliminated by cation-exchange treatment, and the resulting sodium para-styrenesulfonate is purified by recrystallization to remove organic impurities. Here, effects of the sodium bromostyrenesulfonate on the hue are not described.

On the other hand, in the present invention, the purpose to be achieved is reduction of halogen impurities which is strongly desired in the field of electronic materials, in particular, reduction of organohalogen impurity that is difficult to remove, that is, bonded halogen.

According to the patent document described above, it indeed appears that the decreased amount of sodium bromostyrenesulfonate leads to a decrease in amount of bonded halogen. However, when the present inventors analyzed the total halogen content in the high-purity sodium styrenesulfonate by combustion decomposition ion chromatography and the like, a bromine content was detected at more than 400 ppm, a level that is at least 10 times the estimated value from high-performance liquid chromatography. Thereupon, a polystyrenesulfonic acid aqueous solution was actually prepared from the high-purity sodium styrenesulfonate, and the stability was examined. The result showed that the bromide ion concentration markedly increased with time.

Thus, high-purity styrenesulfonic acid compounds in which labile bonded bromine is reduced as much as possible and polymers thereof, and methods for producing the same have been desired. To address the demand, it is necessary that the process and the chemical structure which are involved in incorporation of a bromine content such as bonded bromine be examined in detail rather than making an observation merely on the bromine content.

The present invention has been made in view of the above-described problems, and an object of the present invention is to provide a styrenesulfonic acid compound having a decreased amount of labile bonded bromine, a polymer thereof, and methods for producing the same.

### Means for Solving the Problems

To solve the above-described problems, studies have been extensively conducted with attention focused on 4-(2-bromoethyl)benzenesulfonic acid (hereinafter, sometimes abbreviated as BEBS) which is a precursor of a styrenesulfonic acid compound. As a result, it has been found that BEBS contains nuclear bromides of BEBS (hereinafter, sometimes abbreviated as nuclear-brominated BEBS) including 2-bromo-4-(2-bromoethyl)benzenesulfonic acid which is a previously unreported compound. In addition, it has been found that when control is performed so that the concentrations of an iron content, hydrogen bromide and a moisture content which may be present in the reaction system each fall below a certain level, and a sulfonation reaction is carried out at a specific sulfonating agent concentration and molar ratio of the sulfonating agent to BEBS, high-purity BEBS having a low content of nuclear-brominated BEBS can be produced without impairing productivity, and the amount of bonded bromine in a styrenesulfonic acid compound and a polymer thereof which are derived from the high-purity BEBS can be markedly decreased. In this way, the present invention has been completed.

That is, it is considered that if a stage and a form in which the bonded bromine in the styrenesulfonic acid compound (and polymer thereof) is formed in the process of producing the styrenesulfonic acid compound from styrene are known, conditions for the formation of the bonded bromine can be found.

A reaction process to reach sodium styrenesulfonate from styrene by way of 2-bromoethylbenzene and 4-(2-bromoethyl)benzenesulfonic acid. The thought in the beginning is that during the reaction process, bonded bromine in the styrenesulfonic acid compound (and polymer thereof) is formed by bromine released in the reaction step of adding an alkali such as NaOH to 4-(2-bromoethyl)benzenesulfonic acid at a high temperature of 70° C. to 90° C. to perform vinylation (de-NaBr-ation).

However, studies by the present inventors have shown that bonded bromine (here, nuclear-brominated BEBS) is formed in the step of sulfonating at least one 2-bromoethylbenzene to produce a 4-(2-bromoethyl)benzenesulfonic acid.

Therefore, the present invention has been achieved on the basis of the thought that ingress of bonded bromine into the styrenesulfonic acid compound (and polymer thereof) can be suppressed by identifying a structure of a molecule formed as bonded bromine and a stage in which the molecule is formed.

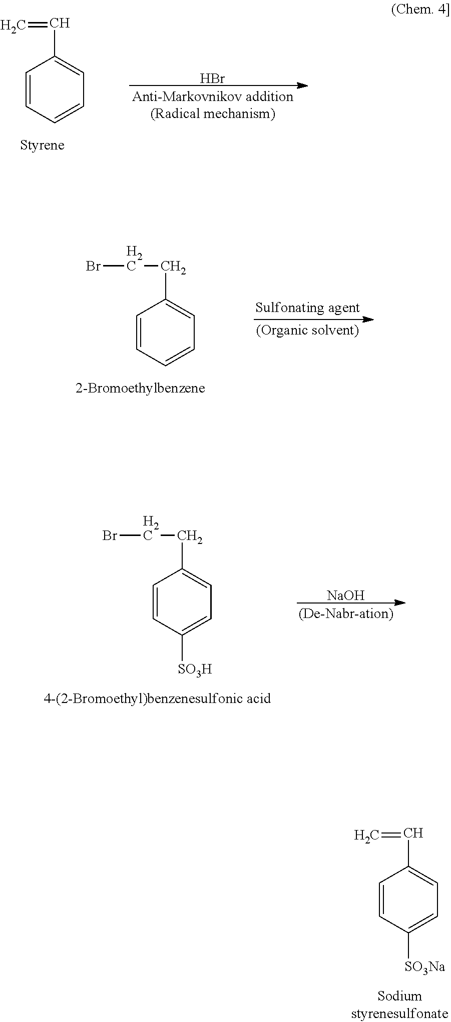

The term “nuclear-brominated BEBS” as used herein refers to BEBS in which at least one bromine atom is covalently bonded to the benzene ring of BEBS (the following general formula (1)), and examples thereof include BEBS of the following general formula (1′) in which one bromine atom is bonded to the benzene ring. Here, the position at which a bromine atom is bonded to the benzene ring is not limited, and examples of the case where one bromine atom is bonded to the benzene ring include 2-bromo-4-(2-bromoethyl)benzenesulfonic acid shown in, for example, FIG. 1 below.

The term “bonded bromine” refers to bromine covalently bonded to the following styrenesulfonic acid compound having a polymerizable vinyl group, and include at least one bromine bonded to the benzene ring of the styrenesulfonic acid compound (the following general formula (2), and an example of one bromine bonded to the benzene ring is given by the following formula (2′). Here, the position at which a bromine atom is bonded to the benzene ring is not limited, and examples of the case where one bromine atom is bonded to the benzene ring include 2-bromo-4-styrenesulfonic acid.

The decreased amount of total bonded bromine leads to a decrease in content of problematic labile bromine in the bonded bromine.

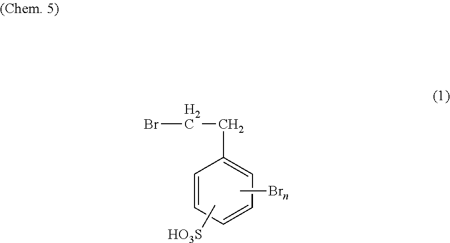

wherein n is an integer of 1 to 3.

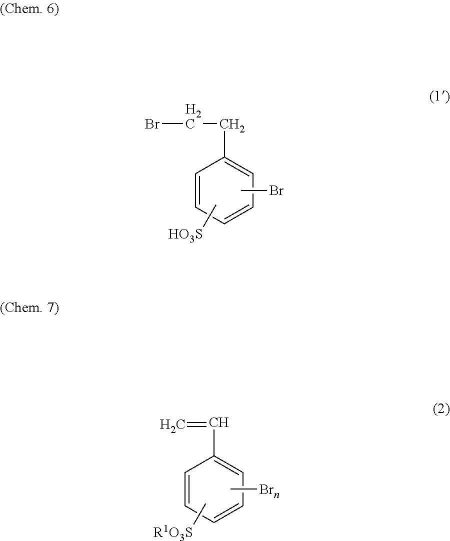

wherein R¹ is as defined in the following general formula (B), and n is an integer of 1 to 3.

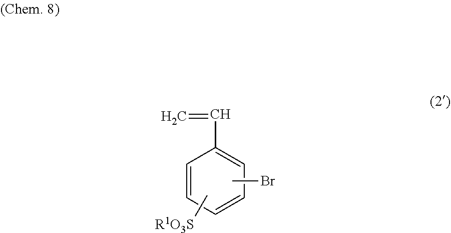

wherein R¹ is as defined in the following general formula (B).

That is, the present invention relates to the following inventions.

(1) A high-purity 4-(2-bromoethyl)benzenesulfonic acid in which a ratio of nuclear-brominated 2-bromoethylbenzenesulfonic acid represented by the following general formula (A) with respect to 4-(2-bromoethyl)benzenesulfonic acid is 0.10% or less [peak area % of nuclear-brominated 2-bromoethylbenzenesulfonic acid when the peak area of 4-(2-bromoethyl)benzenesulfonic acid is defined as 100% is determined by liquid chromatography (LC)]:

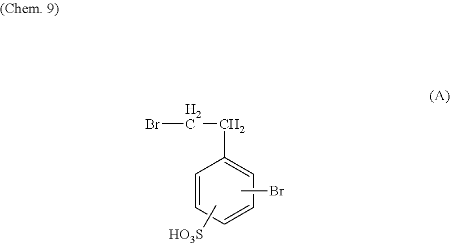

(2) The high-purity 4-(2-bromoethyl)benzenesulfonic acid according to item (1), wherein the nuclear-brominated 2-bromoethylbenzenesulfonic acid is 2-bromo-4-(2-bromoethyl)benzenesulfonic acid.

(3) The high-purity 4-(2-bromoethyl)benzenesulfonic acid according to item (1) or (2), wherein a purity of the 4-(2-bromoethyl)benzenesulfonic acid which is determined by liquid chromatography (LC) is 93 area % or more.

(4) A method for producing the high-purity 4-(2-bromoethyl)benzenesulfonic acid according to any one of items (1) to (3), comprising continuously supplying 2-bromoethylbenzene or a solution of 2-bromoethylbenzene in an organic solvent and anhydrous sulfuric acid or a solution of anhydrous sulfuric acid in an organic solvent to a reactor, the method comprising carrying out the reaction while performing control so that the 2-bromoethylbenzene and the organic solvent each contain an iron content at 5 μg/g or less, hydrogen bromide at 100 ppm or less and a moisture content at 1,000 ppm or less, maintaining a weight percentage of the anhydrous sulfuric acid supplied at 5.00 wt % to 20.00 wt % with respect to the total reaction liquid inside the reactor, and maintaining a molar ratio of the anhydrous sulfuric acid at 0.50 to 2.00 with respect to the 2-bromoethylbenzene inside the reactor.

(5) A method for producing the high-purity 4-(2-bromoethyl)benzenesulfonic acid according to any one of items (1) to (3), comprising continuously supplying anhydrous sulfuric acid or a solution of anhydrous sulfuric acid in an organic solvent to 2-bromoethylbenzene or a solution of 2-bromoethylbenzene in an organic solvent, the method comprising carrying out the reaction while performing control so that the 2-bromoethylbenzene and the organic solvent each contain an iron content at 5 μg/g or less, hydrogen bromide at 100 ppm or less and a moisture content at 1,000 ppm or less, maintaining a weight percentage of the anhydrous sulfuric acid supplied at 20.00 wt % or less with respect to the total reaction liquid inside a reactor, and maintaining a molar ratio of the anhydrous sulfuric acid at 2.00 or less with respect to the 2-bromoethylbenzene inside the reactor.

(6) The production method according to item (4) or (5), wherein the organic solvent is one or more organic solvents selected from the group consisting of a halogenated solvent, a nitrated solvent and an aliphatic hydrocarbon.

(7) The production method according to any one of items (4) to (6), wherein the anhydrous sulfuric acid contains acetic acid or anhydrous acetic acid at 5 wt % to 10 wt % with respect to the anhydrous sulfuric acid.

(8) The production method according to any one of items (4) to (7), wherein the anhydrous sulfuric acid or the solution of anhydrous sulfuric acid in an organic solvent is continuously supplied over 0.5 hours to 7 hours.

(9) The production method according to any one of items (4) to (8), wherein in the reaction, a reaction temperature is 10 to 60° C. and a reaction time is 0.5 hours to 10 hours.

(10) The production method according to any one of items (4) to (9), comprising continuously extracting the reaction liquid in the reaction.

(11) A high-purity styrenesulfonic acid compound, which is represented by the following general formula (B), and has a bonded bromine content of 400 ppm or less as determined by combustion decomposition ion chromatography (CIC):

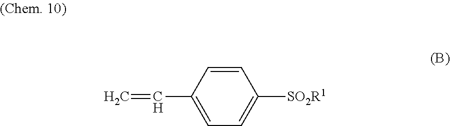

wherein R¹ represents the following general formula (C), the following general formula (D), an amino group, or a chlorine atom:

(Chem. 11)

—OR² (C)
wherein R² represents a substituted or unsubstituted alkyl group having 1 to 6 carbon atoms, a hydrogen atom, an alkali metal, a substituted or unsubstituted ammonium cation, or a substituted or unsubstituted phosphonium cation

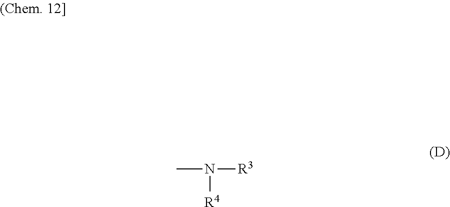

wherein R³ represents a substituted or unsubstituted alkyl group, a hydrogen atom, an alkali metal, or a substituted or unsubstituted ammonium cation, and R⁴ represents a trifluoromethylsulfonyl group, a perfluorobuthylsulfonyl group, a fluorosulfonyl group, a trifluoromethylacetyl group, or a 4-ethenylphenylsulfonyl group,
wherein when R¹ in general formula (B) is an amino group, the amino group may be a primary amino group, a secondary amino group, a tertiary amino group or a quaternary amino group, except for the group of general formula (D).

(12) A high-purity styrenesulfonic acid compound represented by the following general formula (B′), which is sodium 4-styrenesulfonate, lithium 4-styrenesulfonate, potassium 4-styrenesulfonate, ammonium 4-styrenesulfonate, N,N-dimethylcyclohexylamine 4-styrenesulfonate, trioctylamine 4-styrenesulfonate, 4-styrenesulfonyl chloride, 4-styrenesulfonamide, ethyl 4-styrenesulfonate, neopentyl 4-styrenesulfonate, 4-styrenesulfonyl(trifluoromethylsulfonylimide), 4-styrenesulfonyl(perfluorobutylsulfonylimide), 4-styrenesulfonyl(fluorosulfonylimide) or lithium bis-(4-styrenesulfonyl)imide, and has a bonded bromine content of 400 ppm or less as determined by combustion decomposition ion chromatography (CIC):

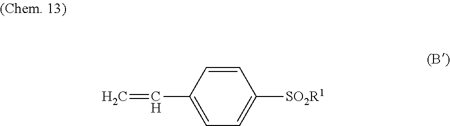

wherein R¹ is as defined in the above general formula (B).

(13) A method for producing the high-purity styrenesulfonate compound according to item (11) or (12), comprising using the high-purity 4-(2-bromoethyl)benzenesulfonic acid according to item (1), or high-purity 4-(2-bromoethyl)benzenesulfonic acid obtained by the production method according to item (4) or (5).

(14) A polystyrenesulfonic acid compound comprising the following repeating structural unit (E), or the following repeating structural unit (E) and the following repeating structural unit (F), wherein a bromide ion concentration in a 10 wt % aqueous solution of the polystyrenesulfonic acid compound is 30 ppm or less when the aqueous solution is held at 70° C. for 20 days:

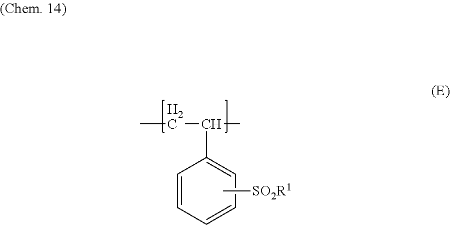

wherein R¹ is the same as R¹ in the general formula (B) described in (11)

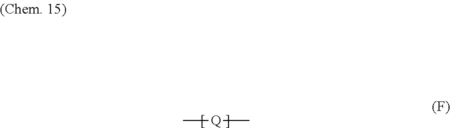

wherein Q represents a repeating structural unit derived from a vinyl monomer that can be copolymerized with a styrenesulfonic acid compound.

(15) The polystyrenesulfonic acid compound according to item (14), wherein a number average molecular weight of the polystyrenesulfonic acid compound is 500 to 5,000,000.

(16) The polystyrenesulfonic acid compound according to item (14) or (15), wherein Q in the repeating structural unit (F) comprises a repeating structural unit derived from a vinyl monomer which is one or a combination of two or more selected from the group consisting of (meth)acrylic acid, a (meth)acrylic acid ester, (meth)acrylamide, N-substituted maleimide, a styrene compound and vinyl pyridine.

(17) The polystyrenesulfonic acid compound according to any one of items (14) to (16), wherein Q in the repeating structural unit (F) comprises a repeating structural unit derived from a crosslinkable monomer which is one or a combination of two or more selected from the group consisting of a substituted styrene compound, a (meth)acrylic acid ester compound, a (meth)acrylamide compound and a N-substituted maleimide compound.

(18) A method for producing the polystyrenesulfonic acid compound according to any one of items (14) to (17), comprising polymerizing the high-purity styrenesulfonic acid compound according to item (11), or a high-purity styrenesulfonic acid compound obtained by the production method according to item (13).

(19) The polystyrenesulfonic acid compound according to any one of items (14) to (17), wherein a bromide ion concentration in a 10 wt % aqueous solution of the polystyrenesulfonic acid compound is 10 ppm or less when the aqueous solution is held at 70° C. for 20 days.

(20) A method for producing the polystyrenesulfonic acid compound according to item (19), comprising chemically treating the polystyrenesulfonic acid compound according to items (14) to (17) through the following step (i) or (ii):
(i) heating a solution of the polystyrenesulfonic acid compound at 90° C. to 110° C. for 5 hours to 30 hours while maintaining the solution at a pH of 13 or more by adding an alkali or an alkali and a reducing agent thereto, followed by purification of the polymer
(ii) adding a reducing agent and a palladium catalyst to a solution of the polystyrenesulfonic acid compound, and heating the mixture at 80° C. to 110° C. for 5 hours to 30 hours, followed by purification of the polymer.

(21) A polystyrenesulfonic acid compound aqueous solution composition in which a content of the polystyrenesulfonic acid compound according to item (14) or item (19), or a polystyrenesulfonic acid compound obtained by the production method according to item (18) is 1 wt % to 60 wt %, a content of a phenolic antioxidant with respect to a net content of the polystyrenesulfonic acid compound is 20 ppm to 2,000 ppm.

(22) The polystyrenesulfonic acid aqueous solution composition according to item (21), wherein the phenolic antioxidant is at least one selected from the group consisting of 2-methoxyphenol, 3-methoxyphenol, 4-methoxyphenol, 2,6-di-tert-butylphenol, 2,4-di-tert-butylphenol, 2,6-di-tert-butyl-4-methylphenol, 4-tert-butylcatechol, hydroquinone and methoxyhydroquinone.

### Advantageous Effects of the Invention

High-purity 4-(2-bromoethyl)benzenesulfonic acid, high-purity styrenesulfonic acid compounds derived therefrom, and polymers thereof according to the present invention, in which the amount of bonded bromine such as nuclear bromides is smaller as compared to conventional products and release of bromine over time is suppressed, thus are extremely useful for electronic material applications such as secondary batteries, capacitors, polymer solid electrolytes, conductive polymers, organic EL elements, photoresists and semiconductor cleaning agents.

### BRIEF DESCRIPTION OF THE DRAWINGS

FIG. 1 is a HPLC chart (enlarged) for a BEBS aqueous solution prepared by a conventional method described in Comparative Example 4, where the horizontal axis represents a dissolution time (min), and the vertical axis represents a peak intensity (mV). In the chart, (A) indicates a peak for 4-(2-hydroxyethyl)benzenesulfonic acid, (B) indicates a peak for a para-isomer of BEBS, (C) indicates a peak for an ortho-isomer of BEBS, (D) indicates a peak for 4-(1-bromoethyl)benzenesulfonic acid, and (E) indicates a peak for 2-bromo-4-(2-bromoethyl)benzenesulfonic acid (nuclear-brominated BEBS).

FIG. 2 is a HPLC chart (enlarged) for a BEBS aqueous solution prepared by a method of the present invention described in Example 1, where the horizontal axis represents a dissolution time (min), and the vertical axis represents a peak intensity (mV). The symbols (A) to (E) in FIG. 2 are as described for FIG. 1.

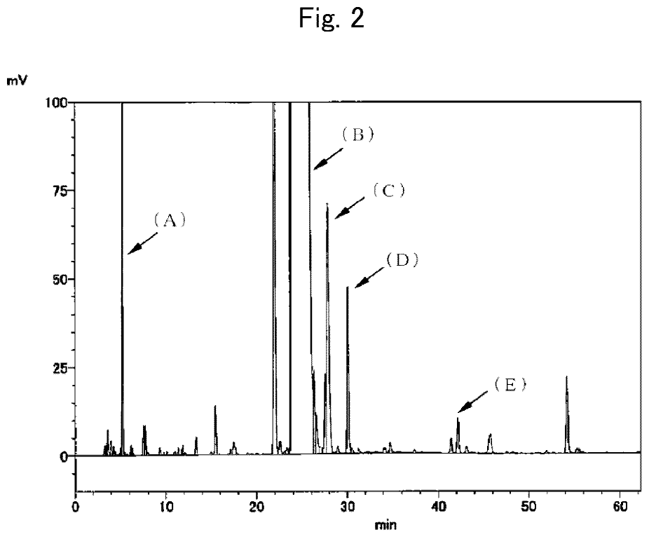

FIG. 3 shows a proton nuclear magnetic resonance spectrum of a sodium salt for the impurity peak (E) shown in FIG. 1, where the horizontal axis represents a chemical shift (ppm), the integer in the vicinity of each peak indicates a carbon type numbered in the chemical structural formula of sodium 2-bromo-4-(2-bromoethyl)benzenesulfonate, and the two-fractional digit value in the vicinity of each peak indicates an area ratio of protons bonded to the carbon.

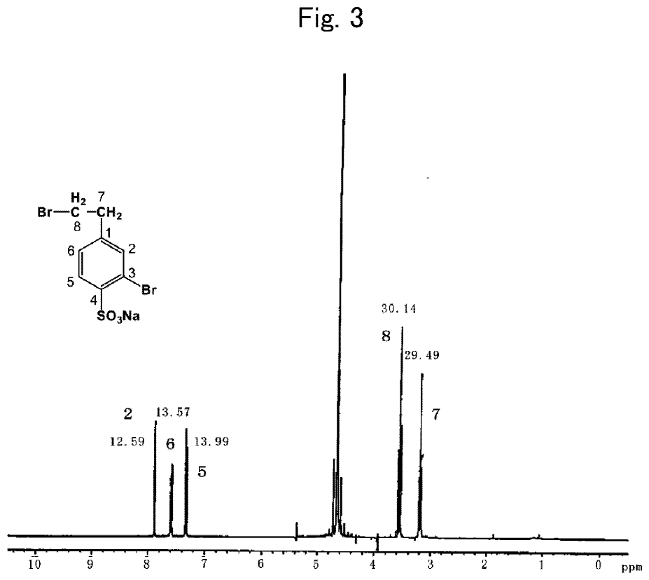

FIG. 4 shows a TOF-MS spectrum of the impurity peak (E) shown in FIG. 1, where the horizontal axis represents a mass/charge ratio m/z (m represents a molecular mass and z represents a charge number), and the vertical axis represents a signal intensity. The mass/charge ratio by mass analysis, the presumed elemental composition, the presumed structure and the desorbed ion species are shown in the middle on the left side of FIG. 4. The enlarged TOF-MS spectrum chart is shown in the middle on the right side of FIG. 4.

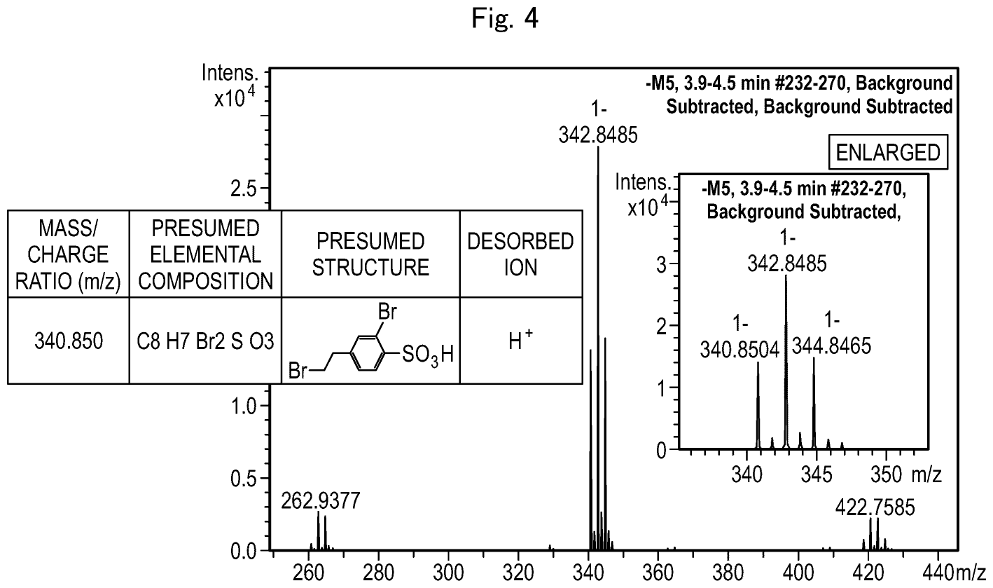

FIG. 5 is a HPLC chart (enlarged) for high-purity sodium styrenesulfonate prepared using BEBS synthesized by a conventional method, which is described in Comparative Example 8, where the horizontal axis represents a dissolution time (min), and the vertical axis represents a peak intensity (mV). The peaks or arrows shown in the chart indicate peaks or the positions of peaks derived (presumed to be derived) from sodium ortho-styrenesulfonate for arrow (a), sodium 4-(2-bromoethyl)benzenesulfonate for arrow (b), sodium meta-styrenesulfonate for arrow (c), sodium bromostyrenesulfonate for arrow (d), and sodium 4-(2-hydroxyethyl)benzenesulfonate (e).

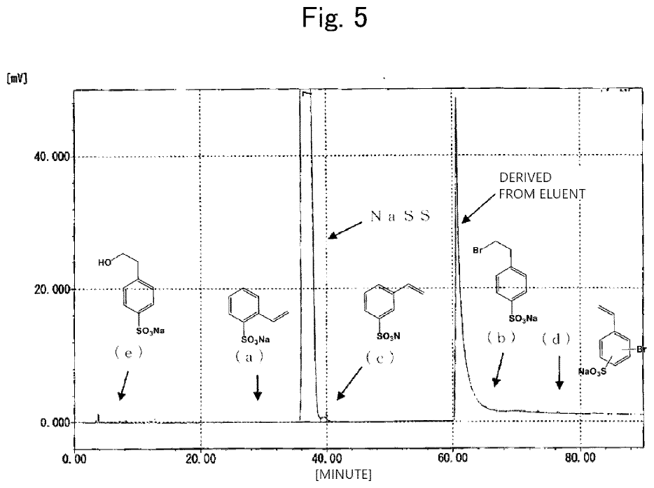

FIG. 6 is a HPLC chart (enlarged) for high-purity sodium styrenesulfonate prepared using BEBS synthesized by the method of the present invention, which is described in Example 12, wherein the horizontal axis represents a dissolution time (min), and the vertical axis represents a peak intensity (mV). Symbol (a) to (e) in FIG. 6 are as described for FIG. 5.

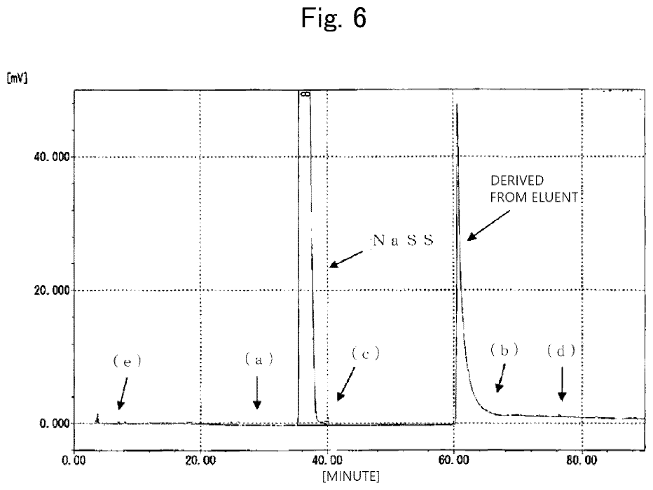

### MODE FOR CARRYING OUT THE INVENTION

The present invention relates to high-purity BEBS in which nuclear-brominated BEBS that may be contained as impurities is at 0.10% or less [peak area % of nuclear-BEBS when the peak area of 4-(2-bromoethyl)benzenesulfonic acid is defined as 100% is determined by liquid chromatography (LC)], a high-purity styrenesulfonic acid compound which is derived from the high-purity BEBS and in which the amount of bonded bromine is 400 ppm or less, and a polymerized product or polymer thereof, and methods for producing the same. It has been found that by decreasing the amount of total bonded bromine in the styrenesulfonic acid compound, the amount of labile bonded bromine which may be present in the styrenesulfonic acid compound can be decreased. In this way, the present invention has been achieved.

The term “area % determined by liquid chromatography (LC)” refers to the percentage (%) of the peak area of nuclear-brominated 2-bromoethylbenzenesulfonic acid when the peak area of 4-(2-bromoethyl)benzenesulfonic acid of interest is defined as 100% in measurement of the 4-(2-bromoethyl)benzenesulfonic acid by liquid chromatography (LC) such as high-performance liquid chromatography (HPLC). Therefore, the phrase “nuclear-brominated BEBS is at 0.10% or less” means that the peak area of nuclear-brominated 2-bromoethylbenzenesulfonic acid when the peak area of 4-(2-bromoethyl)benzenesulfonic acid is defined as 100% is 0.10% or less. That is, the peak area is 1/1,000 or less as compared to that of 4-(2-bromoethyl)benzenesulfonic acid.

The decrease in amount of labile bonded bromine in the bonded bromine is confirmed by forming a polystyrenesulfonic acid aqueous solution from a styrenesulfonic acid compound and monitoring a change in bromide ion concentration as described above.

### 4-(2-Bromoethyl)benzenesulfonic acid (BEBS)

First, a method for producing BEBS having a decreased amount of nuclear-brominated BEBS according to the present invention will be described.

The elementary process for producing BEBS is such that as in a conventional manner, 2-bromoethylbenzene is sulfonated (see, for example, Japanese Patent Publication No. 55-21030), where the 2-bromoethylbenzene is produced by bromination of styrene (see, for example, Japanese Patent Laid-Open No. 1994-172232).

That is, styrene dissolved in a hydrocarbon such as hexane or a halogenated hydrocarbon such as perchloroethylene as shown in the following reaction formula is prepared. The styrene is supplied with hydrogen bromide gas while being irradiated with ultraviolet rays or supplied with a small amount of radical generator such as an azo compound, thereby performing Markovnikov addition to the vinyl group of the styrene. Through this reaction, 2-bromoethylbenzene is obtained. Subsequently, in a dried reactor having acid resistance, the 2-bromoethylbenzene is sulfonated with a sulfonating agent such as anhydrous sulfuric acid (sulfur trioxide), fuming sulfuric acid, concentrated sulfuric acid or chlorosulfuric acid to obtain 4-(2-bromoethyl)benzenesulfonic acid.

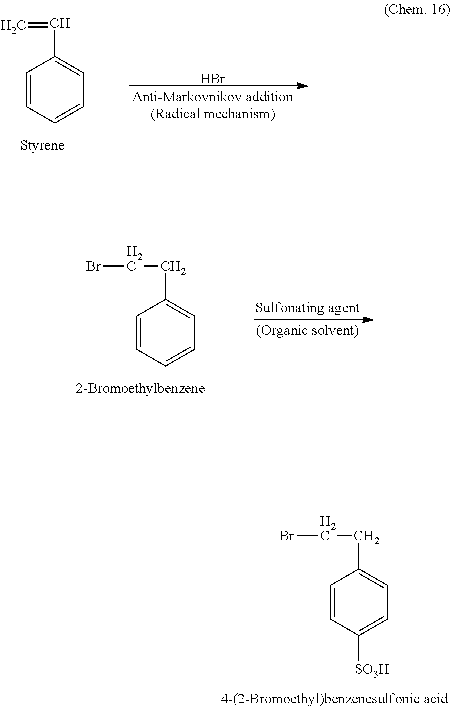

Here, the present invention has the following characteristics (i) to (iv), and thus is distinct from conventional methods.
(i) Control is performed so that hydrogen bromide which may be present in each of 2-bromoethylbenzene and a reaction solvent is at 100 ppm or less.
(ii) Control is performed so that the iron content which may be present in each of 2-bromoethylbenzene and a reaction solvent is at 5 ppm or less.
(iii) Control is performed so that the moisture content which may be present in each of 2-bromoethylbenzene and a reaction solvent is at 1,000 ppm or less.
(iv) Control is performed so that the concentration of the sulfonating agent supplied into the reactor and the molar ratio of the sulfonating agent to the 2-bromoethylbenzene fall within specific ranges.

If these conditions are not met, nuclear-brominated forms may be easily produced as a side product, resulting in an increase in amount of bonded bromine in the styrenesulfonic acid compound, which is derived from the nuclear-brominated forms.

The hydrogen bromide which may be present in 2-bromoethylbenzene may be an unreacted content of hydrogen bromide used as a production raw material, and is controlled to be at 100 ppm or less by performing heating, heating under reduced pressure, bubbling with an inert gas, or washing with pure water, slightly alkaline water or a sodium chloride solution on the 2-bromoethylbenzene, and/or distilling away the hydrogen bromide together with unreacted styrene and the reaction solvent. Normally, it is unlikely that a fresh reaction solvent such as a reagent contains hydrogen bromide. However, in production at a facility such as a chemical plant for producing a material of interest in a practical manner, unreacted raw materials and reaction solvents are reused (recycled), and ingress of hydrogen bromide into the raw materials and solvents. Therefore, when recycled raw materials and reaction solvents are used, the content of hydrogen bromide is analyzed, and controlled to be 100 ppm or less in the same manner as described above.

The iron content which may be present in the reaction system may be iron bromide (III) derived from hydrogen bromide and moisture in the reaction system, the structure of which is unknown. The iron content is controlled to be at 5 ppm or less, preferably 1 ppm or less by subjecting the 2-bromoethylbenzene and the reaction solvent to water washing, purification by distillation, and/or treatment with a cation-exchange resin (for example, AMBERLITE (registered trademark) from Organo Corporation), chelate fiber (for example, CHELEST FIBER (registered trademark) from CHELEST CORPORATION), a cation-exchange filter (for example, KURANGRAFT from KITZ MICRO FILTER CORPORATION), activated carbon (for example, SEITZ AKSJ (registered trademark) from Osaka Gas Chemicals Co., Ltd.) or the like.

The moisture content which may be contained in the 2-bromoethylbenzene and the reaction solvent is derived from water washing of the 2-bromoethylbenzene and the reaction solvent recycled, strongly stimulates production of nuclear-brominated BEBS as a side product, and is controlled to be at 1,000 ppm or less, preferably 500 ppm or less by distillation and/or a drying agent.

As the drying agent, silica gel, zeolite, molecular sieves, calcium chloride, magnesium sulfate, calcium sulfate, sodium sulfate, calcium hydride, phosphorus pentaoxide, alumina and the like are exemplified. They are used to treat the 2-bromoethylbenzene and the reaction solvent, thereby reducing the moisture content in the reaction system.

The BEBS obtained by the above method is typically extracted with water from the reaction solution, then freed of the entrapped reaction solvent and water, and concentrated to obtain a 65 wt % to 75 wt % BEBS aqueous solution, which is used for production of a styrenesulfonic acid alkali metal salt. The organic solvent used in the step of sulfonating the 2-bromoethylbenzene and unreacted 2-bromoethylbenzene is typically collected and recycled. The moisture content particularly easily remains in the collected organic solvent, and it is very important to control the moisture content by the above method.

The reaction solvent is not limited as long as it is inactive against a sulfonating agent, and examples of the reaction solvent that can be used include halogenated solvents such as carbon tetrachloride, 1,2-dichloroethane, methylene chloride, 1,1,2-trichloroethane, chloroform, chlorobenzene, dichlorobenzene, bromobenzene, dibromobenzene and bromohexane, nitrated solvents such as nitromethane and nitrobenzene, and aliphatic hydrocarbons such as hexane, cyclohexane and methylcyclohexane.

In addition, it is important that the concentration of the sulfonating agent supplied to the reactor and the molar ratio of the sulfonating agent to the 2-bromoethylbenzene are controlled to fall within specific ranges. From the viewpoint of suppressing production of nuclear-brominated BEBS, it is preferable that the inside of the reaction system be free or almost free of an iron content, hydrogen bromide and a moisture content, and the lower the substrate concentration, in particular, the lower the concentration of the sulfonating agent, the more preferable, but when practical productivity is considered, the decrease in the substrate concentration is limited. For producing high-purity BEBS without impairing productivity, it is preferable to carry out the reaction while continuously supplying 2-bromoethylbenzene (or a solution thereof in an organic solvent) and a sulfonating agent (or a solution thereof in an organic solvent) simultaneously into the reactor, and a batch system using a tank reactor, or a flow system using a tube-type or tubular reactor can be applied. In the case of mass production, a flow system is more preferable.

In the present invention, when anhydrous sulfuric acid that is most suitable as a sulfonating agent is used, it is preferable that the reaction be carried out at 10° C. to 60° C. for 0.5 hours to 5.0 hours while the anhydrous sulfuric acid concentration within the reactor is maintained at 5.00 wt % to 20.00 wt %, and the molar ratio of anhydrous sulfuric acid to 2-bromoethylbenzene inside the reactor, that is, the ratio between the numbers of moles of the raw materials supplied into the reactor is maintained at 0.50 to 2.00. Here, the anhydrous sulfuric acid concentration is calculated from (weight of anhydrous sulfuric acid supplied to reactor/total reaction liquid weight inside reactor)×100.

For achieving a high reaction conversion rate and selection rate, it is preferable that the reaction be carried out at 20° C. to 50° C. for 0.5 hours to 3.0 hours while the anhydrous sulfuric acid concentration inside the reactor [(weight of anhydrous sulfuric acid supplied to reactor/total reaction liquid weight inside reactor)×100] is maintained at 10.00 wt % to 20.00 wt %, and the molar ratio of the anhydrous sulfuric acid to the 2-bromoethylbenzene inside the reactor (ratio between the numbers of moles of the raw materials supplied into the reactor) is maintained at 0.95 to 1.50.

As a reaction mode other than the method in which raw materials are continuously supplied simultaneously into a reactor, the reaction can be carried out while anhydrous sulfuric acid (or a solution thereof in an organic solvent) is continuously supplied to 2-bromoethylbenzene (or a solution thereof in an organic solvent) and a low anhydrous sulfuric acid concentration inside the reactor system is maintained. In this case, the reaction may be carried out at 10° C. to 60° C. for 0.5 hours to 10.0 hours while the anhydrous sulfuric acid concentration inside the reactor [(weight of anhydrous sulfuric acid supplied to reactor/total reaction liquid weight inside reactor)×100] is increased up to 20.00 wt % over 0.5 hours to 5 hours, and the molar ratio of the anhydrous sulfuric acid to the 2-bromoethylbenzene inside the reactor (ratio between the numbers of moles of the raw materials supplied into the reactor) is increased up to 2.00.

The sulfonating agent is preferably anhydrous sulfuric acid that has high reactivity, can be used in a stoichiometric amount to complete the reaction, and does not generate side products such as hydrochloric acid. In the sulfonation reaction, 5 wt % to 10 wt % of an organic carboxylic acid such as acetic acid or anhydrous acetic acid is preferably added to the sulfonating agent for preventing formation of a sulfone, and the reaction is carried out with sufficient stirring for preventing localization of the sulfonating agent.

The proper amount of the sulfonating agent with respect to the 2-bromoethylbenzene, which is not necessarily the same for all types of sulfonating agents, is preferably 0.50 equivalents to 2.00 equivalents, and more preferably 0.95 equivalent to 1.20 equivalents for achieving a high conversion rate and a high selection rate (suppressing side reactions) when anhydrous sulfuric acid that is most suitable for the present invention is used.

For the purpose of moderating the reactivity of anhydrous sulfuric acid, a compound that forms a complex with anhydrous sulfuric acid, such as a tertiary amine compound such as triethylamine and pyridine, an aprotic polar solvent such as N,N-dimethylformamide, dioxane and dimethylsulfoxide, and a phosphoric acid triester compound such as trimethyl phosphate and triethyl phosphate, may be added in an amount of 0.5 equivalents to 1.5 equivalents with respect to the anhydrous sulfuric acid.

Anhydrous sulfuric acid has extremely high reactivity, and therefore can sufficiently react even at a temperature of lower than 10° C., but the temperature is preferably 10° C. or higher in view of temperature control in production with actual equipment, and more preferably 60° C. or lower in view of the selectivity rate in the reaction.

The nuclear-brominated BEBS that readily comes to mind is an electrophilic substitution reaction of a benzene ring with Br₂ (see, for example, Vollhardt/Schore, Gendai Yuki Kagaku, pp. 698-700, Kagaku-Dojin Publishing Company, INC, published in 2000). That is, it is likely that hydrogen bromide which may be contained in 2-bromoethylbenzene and a recycled solvent is oxidized to form Br₂, and a very small amount of iron which may be present in the reaction system serves as a catalyst, leading to formation of nuclear-brominated BEBS.

However, the present inventors have detailed studies on the reaction conditions, and resultantly found that although coexistence of hydrogen bromide and the iron content certainly causes formation of nuclear-brominated BEBS, it is the moisture content inside the reaction system that has greater effects in actual production processes.

That is, it has been revealed that even though hydrogen bromide and an iron content from raw materials are removed, a large amount of nuclear-brominated BEBS is formed if a moisture content is present in sulfonation of 2-bromoethylbenzene with anhydrous sulfuric acid. The bromine source here may be nothing but bromine bonded to the ethyl group of the 2-bromoethylbenzene, but a relevant reaction mechanism is unknown.

Various isomers may be present in nuclear-brominated BEBS. The impurities found in liquid chromatography analysis of BEBS have been separated and identified, and the results have demonstrated that 2-bromo-4-(2-bromoethyl)benzenesulfonic acid is contained as one of major isomers. It is considered that in production of a styrenesulfonic acid compound using BEBS, the larger the amount of nuclear-brominated BEBS contained in BEBS, the larger the amount of bonded bromine contained in the styrenesulfonic acid compound and labile bonded bromine.

### <Styrenesulfonic Acid Compound>

Next, styrenesulfonic acid compounds of the present invention will be described. The method for producing a styrenesulfonic acid alkali metal salt having a decreased amount of bonded bromine, among styrenesulfonic acid compounds, can be essentially the same as a known method except that high-purity BEBS having a decreased content of nuclear-brominated BEBS is used as a raw material. For example, sodium styrenesulfonate, lithium styrenesulfonate or potassium styrenesulfonate can be produced by making crystallization occur while reacting BEBS and an alkali such as sodium hydroxide, lithium hydroxide or potassium hydroxide in an aqueous solution as described above (e.g., International Publication No. WO 2014/061357, Japanese Patent Laid-Open No. 2015-164911).

The method for producing a styrenesulfonic acid ester having a decreased amount of bonded bromine, among styrenesulfonic acid compounds, is essentially the same as the above method except that high-purity BEBS having a decreased content of nuclear-brominated BEBS is used as a raw material. That is, sodium styrenesulfonate and thionyl chloride are reacted to obtain styrenesulfonyl chloride, which is then esterified with a base such as potassium hydroxide and an alcohol.

The method for producing a styrenesulfonylimide, for example, a 4-styrenesulfonyl(trifluoromethylsulfonylimide) sodium salt having bonded bromine, among styrenesulfonic acid compounds, can be essentially the same as a known method except that high-purity BEBS having a decreased content of nuclear-brominated BEBS is used as a raw material (precursor). For example, a method can be applied in which sodium carbonate, trifluoromethanesulfonamide and the styrenesulfonyl chloride are reacted in an organic solvent (e.g., Japanese Patent Laid-Open No. 2017-132728). A 4-styrenesulfonyl(fluorosulfonylimide) potassium salt can be produced by, for example, a method in which styrenesulfonyl chloride, dipotassium hydrogen phosphate, 4-tert-butylcathecol and dimethylaminopyridine are mixed in acetonitrile at 0° C. in a nitrogen atmosphere, fluorosulfonamide is added thereto, and the mixture is reacted at room temperature for 72 hours. Further, the potassium salt and lithium perchlorate are reacted, whereby a 4-styrenesulfonyl(fluorosulfonylimide) lithium salt can be formed (e.g., Qiang Ma et al.; RSC Advances, 2016, No. 6, pp. 32454-32461). Among bis(styrylsulfonylimide) salts, for example, a lithium salt can be produced by a method in which styrenesulfonyl chloride and styrenesulfonylamide are reacted in the presence of lithium hydride in a dehydrated organic solvent (e.g., Japanese Patent Laid-Open No. 2016-128562).

The method for producing a styrenesulfonic acid amine salt having a decreased amount of bonded bromine, among styrenesulfonic acid compounds, can be essentially the same as a known method except that a styrenesulfonic acid alkali metal salt having a decreased amount of bonded bromine is used as a raw material. For example, a method can be applied in which to a sodium styrenesulfonate aqueous solution is added an aqueous solution of a N,N′-dimethylcyclohexylamine hydrochloric acid salt to perform cation-exchange, a styrenesulfonic acid N,N′-dimethylcyclohexylamine salt is then extracted with an organic solvent such as chloroform, and dried to solidness (e.g., International Publication No. WO 2019/031454).

The method for producing ammonium styrenesulfonate having a decreased amount of bonded bromine, among styrenesulfonic acid compounds, can be essentially the same as a known method except that a styrenesulfonic acid alkali metal salt having a decreased amount of bonded bromine is used as a raw material. For example, when sodium styrenesulfonate and ammonium sulfonate are mixed at 65° C. in methanol, ammonium styrenesulfonate that is soluble in methanol is formed. Sodium styrenesulfonate that is insoluble in methanol is separated by filtration, and the methanol is distilled away, whereby ammonium styrenesulfonate can be produced (e.g., Japanese Patent Laid-Open No. 50-149642).

As the method for producing phosphonium styrenesulfonate having a decreased amount of bonded bromine, among styrenesulfonic acid compounds, essentially a known method can be applied except that a styrenesulfonic acid alkali metal salt having a decreased amount of bonded bromine is used as a raw material. For example, tetrabutylphosphonium bromide and sodium styrenesulfonate are added into water, the mixture is sufficiently stirred to achieve dissolution, and then extracted with an organic solvent, and the extract is washed with pure water, whereby tetrabutylphosphonium styrenesulfonate can be produced (e.g., International Publication No. WO 2015/147749).

### <Polystyrenesulfonic Acid Compound>

For the method for producing a polystyrenesulfonic acid compound according to the present invention, a styrenesulfonic acid compound having a decreased amount of bonded bromine can be used as a monomer. It is also possible to apply a known method to a specific production process.

That is, it is possible to apply common radical polymerization methods and emulsion polymerization methods using a radical polymerization initiator, a photosensitizer, an ultraviolet ray and a radiation (e.g., Hasuike et al.; Shin Kaiteiban Radical Jugo Handbook, 2010, NTS Inc., Lovel Peter A. et al.; Emulsion Polymerization and Emulsion Polymers, 1997, John Wiley & Son Ltd), and controlled radical polymerization methods such as atom transfer radical polymerization (ATRP), reversible addition fragmentation transfer (RAFT) polymerization, iodine transfer polymerization (ITP), stable nitroxyl-mediated polymerization (NMP) and organotellurium-mediated radical polymerization (TERP) (Yamago, et al., Journal of the Society of Rubber Science and Technology, Japan Vol. 82, No. 8, pp. 363-369, 2009; Kamigaito et al., Network Polymer, Vol. 30, No. 5, pp. 234-249, 2009). In addition, when a styrenesulfonic acid ester, among styrenesulfonic acid compounds, is used, anionic polymerization using an organometal catalyst (e.g., Tadaki et al.; Network Polymer, Vol. 38, No. 1, pp. 14-20, 2017) can also be applied.

Among the polymerization methods described above, versatile radical polymerization methods will be described in detail. For example, a solvent and a styrenesulfonic acid compound, and if necessary, a monomer other than a styrenesulfonic acid compounds, which can be copolymerized with a styrenesulfonic acid compound, are added to a reaction vessel. A polymerization control agent such as the stable nitroxyl compound, or a molecular weight modifier such as a mercaptan compound and a radical polymerization initiator such as azo compound are added. The inside of the reaction system is deoxidized, and then subjected to polymerization while being heated to a predetermined temperature, whereby a polymer can be produced which has a desired molecular weight, and is soluble in a solvent. The molecular weight of the polymer on a number average molecular weight basis is 500 to 5,000,000 dalton, but is preferably 500 to 1,000,000 dalton, and more preferably 1,000 to 600,000 dalton, in view of the polymerization property of the styrenesulfonic acid compound.

Among styrenesulfonic compounds, styrenesulfonic acid esters, styrenesulfonic acid amine and lithium styrenesulfonate have high solubility in various solvents, and thus can form a high-concentration solution. For this reason, for example, a monomer solution obtained by adding a photopolymerization initiator, a photosensitizer, and a crosslinkable monomer such as divinylbenzene, and if necessary, a molecular weight modifier and a thickener to such a styrenesulfonic acid compound is injected between transparent glass sheets or films, or infiltrated into a nonwoven fabric, and irradiated with ultraviolet light or the like to be polymerized, whereby a coating film or a crosslinked film of a polystyrenesulfonic acid compound can be easily produced. However, in the case of a crosslinked film, it is difficult to measure a number average molecular weight because the polymer is insoluble in a solvent.

The solvent used in the above reaction is not limited as long as it is capable of dissolving a monomer mixture. Examples thereof include anisole, dimethyl sulfoxide, N,N-dimethylformamide, N-methylpyrrolidone, N,N-dimethylacetamide, dihydrolevoglucosenone, acetonitrile, dioxane, tetrahydrofuran, toluene, benzene, chlorobenzene, xylene, diethyl carbonate, dimethyl carbonate, ethylene carbonate, acetone, methanol, ethanol, propanol, butanol, methoxyethanol, methoxypropanol, propylene glycol monomethyl ether acetate, water, a halogenated alkali metal salt aqueous solution, and mixtures thereof.

The amount of the polymerization solvent used is typically 0 parts by weight to 2,000 parts by weight per 100 parts by weight of all monomers. When a powdered form of a monomer such as a styrenesulfonic acid salt is polymerized, 50 parts by weight to 1,000 parts by weight of a polymerization solvent is typically used.

On the other hand, among styrenesulfonic acid compounds, styrenesulfonic acid esters and specific amine salts, which are liquid or low-melting-point monomers, thus do not necessarily need a reaction solvent. Among styrenesulfonic acid compounds, styrenesulfonic acid esters, which are oil-soluble monomers, thus are miscible with common monomers such as styrene and (meth)acrylic acid esters, and therefore can also be applied to emulsion polymerization, suspension polymerization or dispersion polymerization. For example, a styrenesulfonic acid ester and a monomer that can be copolymerized therewith are emulsified or finely dispersed in water using a nonionic emulsifier, an anionic emulsifier, a cationic emulsifier and/or a water-soluble polymer, and then polymerized with the addition of a radical polymerization initiator, whereby polystyrenesulfonic acid ester fine particles and fine particles modified with styrenesulfonic acid ester structural units can be produced.

The molecular weight modifier is not limited, and examples thereof include disulfide compounds such as diisopropyl xanthogen disulfide, diethyl xanthogen disulfide, diethyl thiuram disulfide, 2,2′-dithiodipropionic acid, 3,3′-dithiodipropionic acid, 4,4′-dithiodibutanoic acid and 2,2′-dithiobisbenzoic acid, mercaptan compounds such as n-dodecyl mercaptan, octyl mercaptan, t-butyl mercaptan, thioglycolic acid, thiomalic acid, 2-mercaptopropionic acid, 3-mercaptopropionic acid, thiosalicyclic acid, 3-mercaptobenzoic acid, 4-mercaptobenzoic acid, thiomalonic acid, dithiosuccinic acid, thiomaleic acid, thiomaleic anhydride, dithiomaleic acid, thioglutaric acid, cysteine, homocysteine, 5-mercaptotetrazolacetic acid, 3-mercapto-1-propanesulfonic acid, 3-mercaptopropane-1,2-diol, mercaptoethanol, 1,2-dimethylmercaptoethane, a 2-mercaptoethylamine hydrochloric acid salt, 6-mercapto-1-hexanol, 2-mercapto-1-imidazole, 3-mercapto-1,2,4-triazole, cysteine, N-acylcysteine, glutathione, N-butylaminoethanethiol and N,N-diethylaminoethanethiol, iodinated hydrocarbons such as iodoform, benzyl dithiobenzoate, 2-cyanoprop-2-yldithiobenzoate, diphenylethylene, p-chlorodiphenylethylene, p-cyanodiphenylethylene, an «-methylstyrene dimer, an organotellurium compound, sulfur, sodium sulfite, potassium sulfite, sodium bisulfite, potassium bisulfite, sodium pyrosulfite, potassium pyrosulfite, and sodium hypophosphite.

The amount of the molecular weight modifier used is typically 0.0 parts by weight to 15.0 parts by weight per 100 parts by weight of all monomers. The molecular weight modifier is an additive that is effective for decreasing the molecular weight and branching of a polymer produced, or improving the homogeneity of a film in production of a polymer electrolyte using a crosslinkable monomer. On the other hand, however, since the molecular weight modifier causes a decrease in polymerization rate and ability to copolymerize, or odors, the molecular weight modifier is not necessarily required for all purposes, and the molecular weight can be adjusted by increasing of the amount of the polymerization initiator, adjustment of the polymerization temperature, or conditions under which the monomer and the polymerization initiator are added.

Examples of the radical polymerization initiator include peroxide compounds such as di-t-butyl peroxide, dicumyl peroxide, t-butylcumyl peroxide, benzoyl peroxide, dilauryl peroxide, cumene hydroperoxide, t-butyl hydroperoxide, 1,1-bis(t-butylperoxy)-3,5,5-trimethylcyclohexane, 1,1-bis(t-butylperoxy)-cyclohexane, cyclohexanone peroxide, t-butyl peroxybenzoate, t-butyl peroxyisobutylate, t-butyl peroxy-3,5,5-trimethylhexanoate, t-butyl peroxy-2-ethylhexanoate, t-butyl peroxyisopropylcarbonate, cumyl peroxyoctoate, potassium persulfate, ammonium persulfate and hydrogen peroxide, azo compounds such as 2,2′-azobis(4-methoxy-2,4-dimethylvaleronitrile), 2,2′-azobis(2,4-dimethylvaleronitrile), 2,2′-azobis(2-methylpropionitrile), 2,2′-azobis(2-methylbutyronitrile), 1,1′-azobis(cyclohexane-1-carbonitrile), 1-[(1-cyano-1-methylethyl) azo]formamide, dimethyl 2,2′-azobis(2-methylpropionate), 4,4′-azobis(4-cyanovaleric acid), 2,2′-azobis(2,4,4-trimethylpentane), 2,2′-azobis {2-methyl-N-[1,1′-bis(hydroxymethyl)-2-hydroxyethyl]propionamide}, 2,2′-azobis {2-(imidazolin-2-yl) propane}dihydrochloride, 2,2′-azobis {2-(2-imidazolin-2-yl) propane}disulfate dihydrate, 2,2′-azobis {2-[1-(2-hydroxyethyl)-2-imidazolin-2-yllpropane]}dihydrochloride, 2,2′-azobis(1-imino-1-pyrrolidino-2-methylpropane) dihydrochloride, 2,2′-azobis(2-methylpropionamidine) dihydrochloride and 2,2′azobis [N-(2-carboxyethyl)-2-methylpropionamidine]tetrahydrate, and photopolymerization initiators such as 4,4′-bis(diethylamino)benzophenone, 2-hydroxy-2-methyl-1-phenyl-1-propanone, ethyl-4-(dimethylamino)-benzoate, [4-(methylphenylthio)phenyl]-phenylmethane, ethylhexyl-4-dimethyl aminobenzoate, benzophenone, methyl-o-benzoyl benzoate, o-benzoylbenzoic acid, 4-methylbenzophenone, 1-hydroxycyclohexyl phenyl ketone, methyl benzoylformate, 2,4,6-trimethylbenzoyldiphenylphosphine oxide, bis(2,4,6-trimethylbenzoyl)-phenylphosphine oxide, 2,2-dimethoxy-2-phenyl acetophenone, 1-[4-(2-hydroxyethoxy)phenyl]-2-hydroxy-2-methylpropane and 2-benzyl-2-dimethylamino-1-(4-morpholinophenyl)-butanone-1,2-methyl-[4-(methylthio)phenyl]-2-morpholino-1-propane. If necessary, a reducing agent such as ascorbic acid, erythorbic acid, aniline, tertiary amine, rongalite, hydrosulfite, sodium sulfite or sodium thiosulfate may be used in combination.

The amount of the radical polymerization initiator used is typically 0.1 parts by weight to 15 parts by weight per 100 parts by weight of all monomers.

While the polymerization conditions are not limited, heating may be performed in an inert gas atmosphere at 20° C. to 120° C. for 4 hours to 50 hours, with an adjustment made as appropriate by a polymerization solvent, a monomer composition and a polymerization initiator species. In the case of photopolymerization, the polymerization may be performed at 10° C. to 60° C. for 0.1 hours to 5 hours with ultraviolet light having a wavelength of 250 nm to 450 nm and a lighting intensity of 20 mW/cm² to 1,000 mW/cm².

The monomer other than a styrenesulfonic acid compound, which is used in production of the polystyrenesulfonic acid compound of the present invention, is not limited as long as it can be copolymerized with a styrenesulfonic acid compound. Examples thereof include styrene compounds such as styrene, chlorostyrene, dichlorostyrene, bromostyrene, dibromostyrene, fluorostyrene, trifluorostyrene, nitrostyrene, cyanostyrene, cx-methylstyrene, p-chloromethylstyrene, p-acetoxystyrene, p-styrenesulfonyl chloride, styrenesulfonyl bromide, styrenesulfonyl fluoride, p-butoxystyrene, 4-vinyl benzoate, 3-isopropenyl-α, α′-dimethylbenzyl isocyanate and vinylbenzyltrimethylammonium chloride, vinyl ether compounds such as butyl vinyl ether, propyl vinyl ether, ethyl vinyl ether, 2-phenyl vinyl alkyl ether, nitrophenyl vinyl ether, cyanophenyl vinyl ether, chlorophenyl vinyl ether and chloroethyl vinyl ether, acrylic acid ester compounds such as methyl acrylate, ethyl acrylate, propyl acrylate, butyl acrylate, pentyl acrylate, hexyl acrylate, decyl acrylate, lauryl acrylate, octyl acrylate, dodecyl acrylate, stearyl acrylate, 2-ethylhexyl acrylate, cyclohexyl acrylate, bornyl acrylate, 2-ethoxyethyl acrylate, 2-butoxyethyl acrylate, 2-hydroxyethyl acrylate, tetrahydrofurfuryl acrylate, methoxyethylene glycol acrylate, ethyl carbitol acrylate, 2-hydroxypropyl acrylate, 4-hydroxybutyl acrylate, 3-(trimethoxysilyl) propyl acrylate, polyethylene glycol acrylate, glycidyl acrylate, 2-(acryloyloxy)ethyl phosphate, 2,2,3,3-tetrafluoropropyl acrylate, 2,2,2-trifluoroethyl acrylate, 2,2,3,3,3-pentafluoropropyl acrylate and 2,2,3,4,4,4-hexafluorobutyl acrylate, methacrylic acid ester compounds such as methyl methacrylate, t-butyl methacrylate, sec-butyl methacrylate, i-butyl methacrylate, i-propyl methacrylate, decyl methacrylate, lauryl methacrylate, octyl methacrylate, dodecyl methacrylate, stearyl methacrylate, cyclohexyl methacrylate, bornyl methacrylate, benzyl methacrylate, phenyl methacrylate, glycidyl methacrylate, polyethylene glycol methacrylate, 2-hydroxyethyl methacrylate, tetrahydrofuryl methacrylate, methoxyethylene glycol methacrylate, ethyl carbitol methacrylate, 2-hydroxypropyl methacrylate, 4-hydroxybutyl methacrylate, 2-(methacryloyloxy)ethyl phosphate, 2-(dimethylamino)ethyl methacrylate, 2-(diethylamino)ethyl methacrylate, 3-(dimethylamino) propyl methacrylate, 2-(isocyanate)ethyl methacrylate, 2,4,6-tribromophenyl methacrylate, 2,2,3,3-tetrafluoropropyl methacrylate, 2,2,2-trifluoroethyl methacrylate, 2,2,3,3,3-pentafluoropropyl methacrylate, 2,2,3,4,4,4-hexafluorobutyl methacrylate, diacetone methacrylate, methacryloxypropyl trimethoxysilane and methacryloxypropyl dimethoxysilane, 1,3-butadiene compounds such as isoprenesulfonic acid, 1,3-butadiene, 2-methyl-1,3-butadiene, 2-chloro-1,3-butadiene, 2,3-dichloro-1,3-butadiene, 2-cyano-1,3-butadiene, 1-chloro-1,3-butadiene, 2-(N-piperidylmethyl)-1,3-butadiene, 2-triethoxymethyl-1,3-butadiene, 2-(N,N-dimethylamino)-1,3-butadiene, N-(2-methylene-3-butenoyl) morpholine and diethyl 2-methylene-3-butenylphosphonate, maleimide compounds such as N-phenylmaleimide, N-(chlorophenyl) maleimide, N-(methylphenyl) maleimide, N-(isopropylphenyl) maleimide, N-(sulfophenyl) maleimide, N-methylphenylmaleimide, N-bromophenylmaleimide, N-naphthylmaleimide, N-hydroxyphenylmaleimide, N—N-methoxyphenylmaleimide, N-carboxyphenylmaleimide, (nitrophenyl) maleimide, N-benzylmaleimide, N-(4-acetoxy-1-naphthyl) maleimide, N-(4-oxy-1-naphthyl) maleimide, N-(3-fluoranthyl) maleimide, N-(5-fluoresceinyl) maleimide, N-(1-pyrenyl) maleimide, N-(2,3-xylyl) maleimide, N-(2,4-xylyl) maleimide, N-(2,6-xylyl) maleimide, N-(aminophenyl) maleimide, N-(tribromophenyl) maleimide, N-[4-(2-benzoimidazolyl)phenyl]maleimide, N-(3,5-dinitrophenyl) maleimide, N-(9-acrydinyl) maleimide, maleimide, N-(sulfophenyl) maleimide, N-cyclohexylmaleimide, N-methylmaleimide, N-ethylmaleimide and N-methoxyphenylmaleimide, fumaric acid diester compounds such as dibutyl fumarate, dipropyl fumarate, diethyl fumarate and dicyclohexyl fumarate, fumaric acid monoester compounds such as butyl fumarate, propyl fumarate and ethyl fumarate, maleic acid diester compounds such as dibutyl maleate, dipropyl maleate and diethyl maleate, maleic acid monoester compounds such as butyl maleate, propyl maleate, ethyl maleate and dicyclohexyl maleate, acid anhydrides such as maleic anhydride and citraconic anhydride, acrylamide compounds such as acrylamide, N-methylacrylamide, N-ethylacrylamide, 2-hydroxyethylacrylamide, N,N-diethylacrylamide, acryloyl morpholine, N,N-dimethylaminopropylacrylamide, isopropylacrylamide, N-methylolacrylamide, sulfophenylacrylamide, 2-acrylamide-2-methylpropanesulfonic acid, 2-acrylamide-1-methylsulfonic acid, diacetone acrylamide and acrylamide alkyl trialkyl ammonium chloride, methacrylamide compounds such as methacrylamide, N-methylmethacrylamide, N-ethylmethacrylamide, 2-hydroxyethylmethacrylamide, N,N-diethylmethacrylamide, N,N-dimethylmethacrylamide, N-methylolmethacrylamide, methacryloyl morpholine, N,N-dimethylaminopropylmethacrylamide, isopropylmethacrylamide, 2-methacrylamide-2-methylpropanesulfonic acid and methacrylamide alkyl trialkyl ammonium chloride, and others such as vinylpyridine, vinyl chloride, vinylidene chloride, vinylpyrrolidone, sulfophenylitaconimide, acrylonitrile, methacrylonitrile, fumaronitrile, o-cyanoethyl acrylate, citraconic acid, vinylacetic acid, vinyl propionate, vinyl pivalate, vinyl versatate, crotonic acid, itaconic acid, fumaric acid, maleic acid, mono 2-(methacryloyloxy)ethyl phthalate, mono 2-(methacryloyloxy)ethyl succinate, mono 2-(acryloyloxy)ethyl succinate, acrolein, vinyl methyl ketone, N-vinylacetamide, N-vinylformamide, vinyl ethyl ketone, vinylsulfonic acid, allylsulfonic acid, dehydroalanine, sulfur dioxide, isobutene, N-vinylcarbazole, vinylidene dicyanide, paraquinodimethane, chlorotrifluoroethylene, tetrafluoroethylene, norbornene, N-vinylcarbazole, acrylic acid and methacrylic acid. Among them, (meth)acrylic acid, (meth)acrylic acid ester, N-substituted maleimide, (meth)acrylamide, styrene compounds and vinylpyridine are preferable when the ability to copolymerize with a styrenesulfonic acid compound, availability, and the like are taken into consideration. The monomer used in production of a crosslinked film and crosslinked particles is not limited, and examples thereof include substituted styrene compounds such as divinylbenzene, bis-(4-styrenesulfonyl)imide and divinylbenzenesulfonic acid, (meth)acrylic acid ester compounds such as polyethylene glycol dimethacrylate, ethylene glycol dimethacrylate, diethylene glycol dimethacrylate, triethylene glycol dimethacrylate, polyethylene glycol diacrylate, ethylene glycol diacrylate, diethylene glycol diacrylate and triethylene glycol diacrylate, (meth)acrylamide compounds such as N,N′-methylenebisacrylamide, N-[tris(3-acrylamidepropoxymethyl)-methyl]acrylamide, N,N-bis(2-acrylamideethyl) acrylamide, N,N′-[oxybis(1,2-ethanediyloxy-3,1-propanediyl)]bisacrylamide, N,N′-1,2-ethanediylbis {N-[2-(acryloylamino)ethyl]acrylamide} and N,N′-methylenebismethacrylamide, and substituted maleimide compounds such as 1,2-bismaleimideethane, 4,4′-bismaleimidediphenylmethane, 1,6-bismaleimidehexane, 1,4-bismaleimidebutane, N,N′-1,4-phenylenedimaleimide and N,N′-1,3-phenylenedimaleimide.

The use ratio of the monomer that can be copolymerized with the styrenesulfonic acid compound is 0.0 mol % to 99.0 mol % with respect to the total amount of monomers. For example, when the polystyrenesulfonic acid compound is used as a dopant for a conductive polymer dispersion, the use ratio of the above-mentioned monomer is 0.0 mol % to 50.0 mol % because the amount of the monomer is preferably small from the viewpoint of the dispersion stability and the conductivity, and preferably large from the viewpoint of the water resistance and the durability of the conductive film.

When the polystyrenesulfonic acid compound is used for a polymer electrolyte film, properties such as an ion-exchange capacity depend on the concentration of sulfonic acid groups, and therefore, the amount of the above-mentioned monomer used is limited, for example, 0.0 mol % to 30 mol %.

On the other hand, for example, when the polystyrenesulfonic acid compound is used as fine spacer particles for a liquid crystal display, the use ratio of the above-mentioned monomer is 50.0 mol % to 99.0 mol % because the monomer is a main component and the styrenesulfonic acid compound is a stabilizer for use in production of the fine particles, that is, a minor component (secondary component).

The type of copolymerization is not limited, and not only a random copolymer, an alternate copolymer and a graft copolymer can be produced, but also a block copolymer can be produced when the above-described controlled polymerization method is applied.

BEBS having a reduced amount of nuclear-brominated BEBS, which is produced according to the present invention, is extremely useful as a precursor for use in production of a styrenesulfonic acid compound having a reduced amount of bonded bromine, and a polymer thereof. The polymer of the styrenesulfonic acid compound may be directly used, or may be subjected to the following chemical treatment to further decrease the amount of bonded bromine.

That is, to an aqueous solution of the polystyrenesulfonic acid compound obtained as described above, an alkali and a reducing agent are added, and the mixture is heated at 80° C. to 150° C. for 5 hours to 30 hours while the solution is maintained at a pH of 13 or more, whereby bonded bromine present in the polymer is released, followed by purification of the polymer. It is more preferable to perform heat treatment at 90° C. to 110° C. for 10 hours to 25 hours for decreasing the amount of bonded bromine without degradation, such as coloring, of the polymer.

The atmosphere in which the chemical treatment is performed may be an air atmosphere, but is preferably an inert gas atmosphere such as a nitrogen or argon atmosphere from the viewpoint of suppressing deactivation of the reducing agent and degradation of the polystyrenesulfonic acid compound. Examples of the alkali include sodium hydroxide, potassium hydroxide, lithium hydroxide, tetramethylammonium hydroxide, and tetraethylammonium hydroxide, and examples of the reducing agent include sodium sulfite, rongalite, hydrosulfite, sodium thiosulfate, and sodium hypophosphite.

The amount of the reducing agent added is 0.5 times to 1.5 times that of the alkali on a molar basis. Examples of the other chemical treatment method include those with a combination of a reducing agent such as sodium formate or hydrazine and a palladium-on-carbon catalyst. For example, 1.0 wt % to 5.0 wt % of a reducing agent with respect to the net content of the polystyrenesulfonic acid compound and 1.0 wt % to 20 wt % of palladium on carbon (with a Pd content of 5 wt %) with respect to the reducing agent are added, and the mixture is allowed to stand at 80° C. to 110° C. for 5 hours to 30 hours.

As a method for purification after the treatment, a method using a cation-exchange resin, an anion-exchange resin, a cation-exchange filter, an anion-exchange filter, chelate fiber, an ultrafiltration membrane and activated carbon, or reprecipitation purification can be applied, and a cation-exchange resin, an anion-exchange resin and an ultrafiltration membrane are preferably used when applicability to a high-viscosity polymer aqueous solution is considered.

The aqueous solution of the polystyrenesulfonic acid of the present invention can be directly used for various purposes, but it is preferable that as a stabilizer, a phenolic antioxidant be added at 20 ppm to 2,000 ppm with respect to the net content of the polystyrenesulfonic acid for suppressing cleavage of polymer chains in storage for a long period. The phenolic antioxidant is not limited, but is preferably one that dissolves in a polystyrenesulfonic acid aqueous solution, and examples thereof include 2-methoxyphenol, 3-methoxyphenol, 4-methoxyphenol, 2,6-di-tert-butylphenol, 2,4-di-tert-butylphenol, 2,6-di-tert-butyl-4-methylphenol, 4-tert-butylcatechol, hydroquinone, methoxyhydroquinone, and ethoxyhydroquinone.

When the polystyrenesulfonic acid of the present invention is neutralized with ammonia, amine, tetramethylammonium hydroxide, tetraethylammonium hydroxide or the like, and used as an ammonium salt, that is, the aqueous solution is at a neutral or higher pH, the antioxidant is not necessarily added.

A styrenesulfonic acid compound having a decreased amount of bonded bromine and a polymer thereof according to the present invention, which have a decreased amount of labile bonded bromine that is released under mild conditions, thus are extremely useful, in particular, for electronic material applications such as members for batteries, members for organic EL, members for photoresists, dispersants also serving as dopants for conductive polymers and carbon nanotubes, dispersants for chemical mechanical polishing slurry and semiconductor cleaning agents.

### EXAMPLES

The present invention will be described in further detail by way of Examples below, which should not be construed as limiting the present invention.

In Examples below, compounds were analyzed and evaluated under the following conditions.

### <Analysis of Iron Content in BEB and Organic Solvent by Inductively Coupled Plasma Atomic Emission Spectroscopy (ICP-AES)>

Apparatus: Nexlon (registered trademark) 300S manufactured by PerkinElmer
Sample preparation: About 0.1 g of the sample was taken in a 25 ml plastic measuring flask while being precisely weighed, 1 ml of 68% high-purity nitric acid was added, and the mixture was diluted with ultrapure water to obtain a measurement sample.

### <Analysis of Moisture Content in BEB and Organic Solvent by Karl Fischer Method>

Apparatus: MKC-610 manufactured by Kyoto Electronics Manufacturing Co., Ltd.
Anode solution: KEMAQUA Anode Solution AGE (manufactured by Kyoto Electronics Manufacturing Co., Ltd.)
Cathode solution: KEMAQUA Cathode Solution CGE (manufactured by Kyoto Electronics Manufacturing Co., Ltd.)

### <Analysis of the Hydrogen Bromide in BEB and Organic Solvent by Ion Chromatography (IC)>

Apparatus: IC-2001 manufactured by Tosoh Corporation
Column: TSKgel (registered trademark) SuperIC-AP
Detector: Electric conductivity, flow rate: 0.8 ml/min, column temperature: 40° C.
Calibration curve: Absolute calibration curve method using an anion standard solution
Sample preparation: The sample was extracted with ultrapure water under shaking, and then centrifuged, and the aqueous layer was passed through a pretreatment cartridge (TOYOPAK (registered trademark) ODS M manufactured by Tosoh Corporation) to obtain a measurement sample. The dilution factor was adjusted to, for example, to a sample/ultrapure water of 2 ml/200 ml to 2 ml/4 ml by the HBr concentration.

### <Measurement of BEB Conversion Rate by High-Performance Liquid Chromatography (HPLC)>

The conversion rate (area %) of BEB in the sulfonation reaction of BEB was measured under the following conditions.
Apparatus: manufactured by Tosoh Corporation
Column: TSKgel (registered trademark) ODS-80TM (4.6 mm I.D.×25 cm)
Eluent: A) 20 mM NaH2P04 (pH=2.4) aqueous solution/acetonitrile=90/10 volume ratio
B) 20 mM NaH2P04 (pH=2.4) aqueous solution/acetonitrile=60/40 volume ratio
Gradient: A→B (linear-gradient for 60 minutes, followed by continuation with solution B for 30 minutes)
Detector: Ultraviolet ray UV 230 m, column temperature: 25° C., flow rate: 0.8 ml/min, injection amount: 20 μl

### <Analysis of Concentration of BEBS Aqueous Solution by High-Performance Liquid Chromatography (HPLC)>

The concentration of the BEBS aqueous solution was analyzed under the following conditions.
Column: TSKgel (registered trademark) ODS-80TsQA (4.6 mm I.D.×25 cm)
Eluent: Water/acetonitrile=80/20 volume ratio+0.1 wt % trifluoroacetic acid
Detector: ultraviolet ray UV 230 m
Column temperature: 40° C., flow rate: 0.8 ml/min
Calibration curve: Absolute calibration curve method using a standard substance obtained by collecting crystals from the BEBS aqueous solution prepared in Example 2

### <Analysis of Impurities in BEBS by High-Performance Liquid Chromatography (HPLC)>

The nuclear-brominated form (area %) in the BEBS aqueous solution was analyzed under the following conditions.
Apparatus: manufactured by Tosoh Corporation
Column: TSKgel (registered trademark) ODS-80TM (4.6 mm I.D.×25 cm)
Eluent: A) 20 mM NaH2P04 (pH=2.4) aqueous solution/acetonitrile=90/10 volume ratio
B) 20 mM NaH2P04 (pH=2.4) aqueous solution/acetonitrile=60/40 volume ratio
Gradient: A→B (linear-gradient for 60 minutes)
Detector: Ultraviolet ray UV 230 m, column temperature: 25° C., flow rate: 0.8 ml/min, injection amount: 20 μl
Sample preparation: BEBS aqueous solution (concentration 69.0 to 73.0 wt %) 5 mg/eluent 1 ml

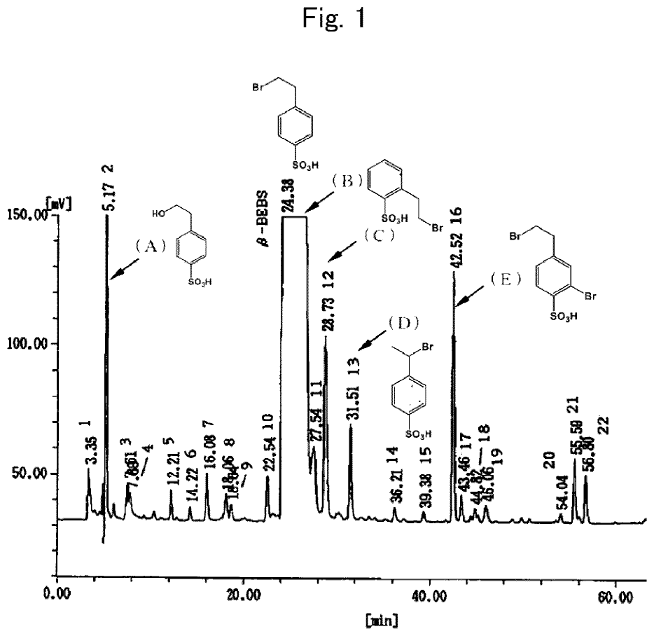

UK components in BEBS produced by a conventional method (Comparative Example 4 shown below), that is, peaks (A), (C), (D) and (E) in FIG. 1, were identified in advance by the method described below. The components were separately taken in columns, the sulfonic acid groups were methyl-esterified with diazomethane, and gas chromatography-mass spectrometry (M-80B manufactured by Hitachi, Ltd.), Fourier transform infrared analysis (System 2000 manufactured by PerkinElmer), organic elementary analysis (CHN Corder MT-3 manufactured by Yanako Technical Science Co. Ltd.) and nuclear magnetic resonance analysis (VXR-300 manufactured by Varian Company) were performed to identify the components. For (E), the esterification was not performed, and the component was directly identified by TOF-MS.

### <Identification of Nuclear-Brominated BEBS by Time-of-Flight Mass Spectrometry>

TOF-MS measurement was performed on the peak (E) detected in HPLC.
Apparatus: Bruker Daltonics microTOF
Ion source: ESI
Measurement mode: Negative mode
Sample preparation: The sample was dissolved in methanol, followed by dilution to a methanol/water volume ratio of 1/1.

### <Analysis of CISS Toluene Solution Concentration by Proton Nuclear Magnetic Resonance Method (¹H-NMR)>

The ¹H-NMR of a styrenesulfonyl chloride (CISS) solution was measured under the following conditions.
Apparatus: AV-400M manufactured by Bruker
Solvent: Deuterated dimethyl sulfoxide
From the area ratio of two protons at meta positions of toluene to one proton of CH₂=in the CISS vinyl group CH₂═CH—, the CISS concentration was calculated by the following expression.
CISS concentration=100×202.65/202.65+92× toluene area ratio/2)

### <Analysis of ETSS by Gas Chromatography (GC)>

The GC purity (area %) of ethyl styrenesulfonate (ETSS) was analyzed under the following conditions.
Apparatus: GC-2014 manufactured by Shimadzu Corporation
Column: NEUTRABOND-1 (¢0.32 mm×30 m, 0.4 μm)
Injection: 220° C., injection amount 0.2 μl
Carrier gas: Helium, linear velocity: 30 cm/min, sprit ratio: 100
Detector: FID, 250° C.
Heating condition: held at 80° C. for 10 minutes, then heated at 5° C./min to 250° C., and held for 6 minutes
Sample: Neat

### <Analysis of NaSS by High-Performance Liquid Chromatography (HPLC)>

Various organic impurities and isomers which may be contained in sodium 4-styrenesulfonate were analyzed under the following conditions which are the same as in a patent document (International Publication No. WO 2014/061357).
Apparatus: manufactured by Tosoh Corporation
Column: TSKgel (registered trademark) ODS-80TM (4.6 mm I.D.×25 cm)
Eluent: A) 5 vol % acetonitrile aqueous solution (containing 0.1% trifluoroacetic acid)
B) 20 vol % acetonitrile aqueous solution (containing 0.1% trifluoroacetic acid)
Gradient: Liquid A 100% (0-55 min)→liquid B 100 (55-95 min)
Detector: Ultraviolet ray UV 230 m, column temperature: 25° C., flow rate: 0.8 ml/min, injection amount: 20 μl
Sample preparation: The sample is dissolved in the eluent A to prepare a solution with an as-prepared concentration of 0.5 mg/1 ml.

### <Measurement of Net Content of Sodium Styrenesulfonate>

By redox titration, active double bonds were quantified as a content of sodium styrenesulfonate (including para-isomers as well as ortho- and met-isomers) in the sample.

### (1) Reagents

1) Bromine solution: 22.00 g of potassium bromide and 3.00 g of potassium bromate were dissolved in pure water such that the total amount was 1,000 ml.
2) Sulfuric acid aqueous solution (concentrated sulfuric acid/pure water=1/1 on a volume basis)
3) Potassium iodide aqueous solution (200 g/L)
4) 0.1 mol/L Sodium thiosulfate aqueous solution
5) Starch aqueous solution: 6.00 g of starch was dissolved in pure water such that the total amount was 1,000 ml.

### (2) Procedures

1) 20 g of the sample is taken in a weighing bottle while being weighed to one decimal place in milligram.
2) The sample is washed off into a 500 ml measuring flask with pure water, and the liquid amount is adjusted to about 400 ml.
3) A magnetic rotor is put in the liquid, and stirring is performed to dissolve the sample.
4) The rotor is removed, pure water is added so that the liquid level matches the marked line, and the liquid is shaken to obtain a test liquid.
5) To a 500 ml conical flask with a stopper which contains 200 ml of pure water, 25 ml of a bromine solution is added.
6) 5 ml of the test liquid is added, 10 ml of a sulfuric acid aqueous solution is then added, the flask is stoppered, and the mixture is left to stand for 20 minutes.
7) 10 ml of a potassium iodide aqueous solution is promptly added, and the mixture is left to stand for 20 minutes.
8) The reaction liquid is titrated with a sodium thiosulfate aqueous solution, 1 ml of a starch solution is added as an indicator after the yellowness of the solution is reduced, and the titration is continued until the developed blue color from iodo-starch is eliminated.
9) Meanwhile, as a blank test, 200 ml of pure water is added, 25 ml of a bromine solution is added to a conical flask with a stopper, 10 ml of a potassium iodide aqueous solution and 10 ml of a sulfuric acid aqueous solution are promptly added, and the procedure 8) is carried out.

### (3) Calculation

The content of sodium styrenesulfonate is calculated by the following expression.

$$A = 100 \times [ 0.01031 \times ( a - b ) \times f / ( S \times 5 / 500 )$$

A: content of sodium styrenesulfonate (%)
a: sodium thiosulfate aqueous solution required for blank test (ml)
b: sodium thiosulfate aqueous solution required for main test (ml)
f: titer of sodium thiosulfate aqueous solution
S: sample amount (g)

### <Analysis of Halogen in Styrenesulfonic Acid Compound by Combustion Decomposition Ion Chromatography>

The bromine content and the chlorine content in the styrenesulfonic acid compound and the polystyrenesulfonic acid were quantified under the following conditions.
Combustion apparatus: AQF-2100H manufactured by Mitsubishi Chemical Analytech Co., Ltd.
Combustion temperature: inlet=900° C., outlet=1,000° C.
IC apparatus: IC-2010 manufactured by Tosoh Corporation
Column: TSK-guard column Super IC-AHS+TSK-gel super IC-Anion HS
Eluent: Carbonate buffer
Absorption liquid: 900 ppm hydrogen peroxide water
Detector: Electric conductivity
Flow rate: 1.5 ml/min, temperature: 40° C.
Measurement mode: Suppressor type
Calibration curve: Absolute calibration curve method using an anion standard solution

### <Analysis of Halogen Ions in Polystyrenesulfonic Acid Aqueous Solution by Ion Chromatography>

Halogen ions in the polystyrenesulfonic acid aqueous solution were quantified under the following conditions.
Apparatus: IC-2001 manufactured by Tosoh Corporation
Column: TSKgel (registered trademark) Super IC-AP
Detector: Electric conductivity
Sample preparation: 20 g of the sample was diluted with ultrapure water to adjust the concentration to 20 mg/ml, and the polymer component was then removed with an ultrafiltration cartridge (molecular weight cut off: 3,000 or 10,000) to obtain an analysis sample.

### <Quantification of Moisture Content in 4-Styrenesulfonic Acid Salt>

About 2 g of the sample was taken in a weighing bottle (55 mm in diameter and 30 mm in height) while being weighed to one decimal place in milligram, followed by drying in a dryer (105±5° C.) for 90 minutes. The sample was immediately transferred into a desiccator, and allowed to cool to room temperature, the mass of the weighing bottle was then measured to one decimal place in milligram, and the moisture content was calculated from the following expression.

$$Moisture content ( wt \% ) = 100 \times ( a - b ) / S$$

a: weight (g) of sample and weighing bottle before drying, b: weight (g) of sample and weighing bottle after drying, S: sample amount (g)

### <Quantification of Bromide Ions in 4-Styrenesulfonic Acid Compound>

Apparatus: IC-2010 manufactured by Tosoh Corporation
Column: TSKgel (registered trademark) guard column Super IC-AHS (4.6 mm I.D.×1 cm)+TSKgel SuperIC-Anion HS (4.6 mm I.D.×10 cm)
Column temperature: 40° C., injection amount: 30 μl, flow rate: 1.5 ml/min
Eluent: Carbonate buffer (7.5 mM-NaHCO₃+0.8 mM-Na2CO3)
Preparation of styrenesulfonic acid ester compound sample: 5 ml of ultrapure water and 5 g of the sample were taken in a screw-top test tube, extracted under shaking for 30 minutes, and then centrifuged (2,800 rpm, 30 min). The aqueous layer was passed through a pretreatment cartridge (TOYOPAK ODSM) to obtain a measurement sample.
Preparation of styrenesulfonic acid salt compound sample: The solid sample was dissolved in and diluted 10-fold with ultrapure water, and passed through a pretreatment cartridge (TOYOPAK (registered trademark) ODSM) to obtain a measurement sample.
Calibration curve: Absolute calibration curve method using a standard solution

### <Analysis of Polymerization Conversion Rate of Styrenesulfonic Acid Ester and Molecular Weight of Formed Polymer by Gel Permeation Chromatography (GPC)>

The conversion rate and the molecular weight on an area basis were under the following conditions.
Model: HLC-8320GPC manufactured by Tosoh Corporation
Column: TSK guard column Super AW-H+TSK Super AW-6000+TSK Super AW-4000+TSK Super AW-2500
Eluent: N,N-dimethylformamide (lithium bromide 10 mM)
Column temperature: 40° C., flow rate: 0.5 ml/min
Detector: RI detector, injection amount: 10 μl
Calibration curve: prepared from weight average molecular weights and elution times using Standard Polystyrene Kit PSt Quick C, D, E manufactured by Tosoh Corporation.
Conversion rate: The polymerization conversion rate was calculated by the following expression from a peak area (a) derived from a monomer and a peak area (b) derived from a polymer.

$$Conversion rate ( area \% ) = 100 \times [ 1 - \{ a / ( a + b ) \} ]$$

### <Analysis of Polymerization Conversion Rate of Styrenesulfonic Acid Salt and Molecular Weight of Formed Polymer by Gel Permeation Chromatography (GPC)

The conversion rate and the molecular weight on an area basis were under the following conditions.
Model: HLC-8320GPC manufactured by Tosoh Corporation
Column: TSK guard column AW-H+TSK AW6000+TSK AW3000+TSK AW2500
Eluent: 0.05 M sodium sulfate aqueous solution/acetonitrile=65/35 volume ratio
Flow rate: 0.6 ml/min, injection amount: 10 μl, column temperature: 40° C.
Detector: UV detector (wavelength 230 nm)
Calibration curve: prepared from peak top molecular weights at peak tops (3K, 15K, 41K, 300K, 1000K, 2350K, 5000K) and elution times using standard sodium polystyrenesulfonate (manufactured by Sowa Kagaku Ltd.)
Conversion rate: The polymerization conversion rate was calculated by the following expression from a peak area (a) derived from a monomer and a peak area (b) derived from a polymer.

$$Conversion rate ( area \% ) = 100 \times [ 1 - \{ a / ( a + b ) \} ]$$

### <Reagents Used>

2-Bromoethylbenzene: manufactured by Tosoh Finechem Corporation, purity 99.1%
1,2-Dichloroethane: manufactured by Tosoh Corporation, purity 99.9%
Anhydrous sulfuric acid: Nisso Sulfan (registered trademark) manufactured by Nisso Metallochemical Co., Ltd., purity 99.4%
Acetic acid: manufactured by Tokyo Chemical Industry Co., Ltd., purity>99.5%
Anhydrous barium hydroxide: manufactured by FUJIFILM Wako Pure Chemical Corporation
Triethyl orthoacetate: manufactured by Tokyo Chemical Industry Co., Ltd., purity>96%
t-Butylcatechol: manufactured by FUJIFILM Wako Pure Chemical Corporation, purity 98%
Potassium t-butoxide: manufactured by Tokyo Chemical Industry Co., Ltd., purity>97%
N,N-dimethylformamide: manufactured by Tokyo Chemical Industry Co., Ltd., purity>99.5%
Irganox 1010: manufactured by BASF Japan Ltd.
Thionyl chloride: manufactured by Tokyo Chemical Industry Co., Ltd., purity>98%
Neopentyl alcohol: manufactured by Tokyo Chemical Industry Co., Ltd., purity>98%
Trifluoromethanesulfonamide: manufactured by Tokyo Chemical Industry Co., Ltd., purity>98%
Pyridine: manufactured by Tokyo Chemical Industry Co., Ltd., purity>99%
4-Dimethylaminopyridine: manufactured by Tokyo Chemical Industry Co., Ltd., purity>98%
Sodium carbonate: manufactured by Tokyo Chemical Industry Co., Ltd., purity>99%
Ethyl acetate: manufactured by Tokyo Chemical Industry Co., Ltd., purity>98%
Toluene: manufactured by Tokyo Chemical Industry Co., Ltd., purity>99.5%
Sodium formate: manufactured by FUJIFILM Wako Pure Chemical Corporation, purity>95%
Palladium on carbon: manufactured by FUJIFILM Wako Pure Chemical Corporation, Pd content 5 wt %

### Abbreviations of Compounds

BEB: 2-bromoethylbenzene
BEBS: 4-(2-bromoethyl)benzenesulfonic acid
NaSS: Sodium 4-styrenesulfonate
LiSS: Lithium 4-styrenesulfonate
Poly NaSS: poly(sodium 4-styrenesulfonate)
Poly LiSS: poly(lithium 4-styrenesulfonate)
PSS: poly(4-styrenesulfonic acid)
CISS: 4-styrenesulfonyl chloride
ETSS: ethyl 4-styrenesulfonate
Poly ETSS: poly(ethyl 4-styrenesulfonate)
NPSS: neopentyl 4-styrenesulfonate
Poly NPSS: poly(neopentyl 4-styrenesulfonate)
TfNS-Na: 4-styrenesulfonyl(trifluoromethylsulfonylimide) sodium
Poly TfNS-Na: poly [4-styrenesulfonyl(trifluoromethylsulfonylimide) sodium]
Poly TfNS-H: poly [4-styrenesulfonyl(trifluoromethylsulfonylimide)]
BVBSI-Li: lithium bis-(4-styrenesulfonyl)imide

### Example 1: Production of 4-(2-bromoethyl)benzenesulfonic acid (BEBS) (1)

A 1 L four-necked glass flask equipped with a reflux cooling tube, a nitrogen inlet tube, a thermometer insertion tube and a drop funnel was charged with 233.80 g (1.25 mol) of 2-bromoethylbenzene (manufactured by Tosoh Finechem Corporation) and 250.30 g of 1,2-dichloroethane (manufactured by Tosoh Corporation). The drop funnel was charged with a mixed solution of 111.30 g (1.38 mol) of anhydrous sulfuric acid, 8.00 g (0.13 mol) of acetic acid and 250.10 g of 1,2-dichloroethane. Note that the 2-bromoethylbenzene was washed with pure water, then treated with CHELEST FIBER (registered trademark) IRY-LC12 (manufactured by CHELEST CORPORATION) having a cation exchange property, dried with molecular sieves, and confirmed to contain an iron content at less than 1 ppm, a hydrogen bromide content at 20 ppm and a moisture content at 20 ppm in advance. The 1,2-dichloroethane was dried with molecular sieves, and confirmed to contain a moisture content at 63 ppm, an iron content at less than 1 ppm and a hydrogen bromide content at less than 1 ppm in advance. In a nitrogen atmosphere, the mixture was sufficiently stirred with a magnetic rotor, and a mixed solution of anhydrous sulfuric acid and anhydrous acetic acid was added dropwise over 1 hour while the internal temperature was controlled to be 30 to 40° C. After the dropwise addition, the mixture was aged at 40° C. for 1 hour. The concentration of the anhydrous sulfuric acid supplied to the reactor was 0.00 to 12.96 wt % until completion of the reaction after the start of the reaction, and the molar ratio of the anhydrous sulfuric acid to the 2-bromoethylbenzene was 0.00 to 1.10 (the reaction conversion rate of the 2-bromoethylbenzene after completion of the aging is 97.6%).

After completion of the aging, 162.80 g of pure water was added while an internal temperature of 30 to 40° C. was maintained. The mixture was sufficiently stirred, and then left to stand, and a downside aqueous solution containing BEBS was collected using a separating funnel. Remaining 1,2-dichloroethane and water were distilled away from the aqueous solution with a rotary evaporator to obtain 437.32 g of a concentrated BEBS aqueous solution. The BEBS concentration measured by HPLC was 70.4 wt %, that is, the yield with respect to the charge 2-bromoethylbenzene was 92.8%.

The BEBS aqueous solution was analyzed by HPLC, and the results showed that the purity was 96.2 area %, and the peak area of nuclear-brominated BEBS when the peak area of BEBS is defined as 100 was 0.01%. It is thus evident that as shown in Table 1, the BEBS aqueous solution has a smaller content of nuclear-brominated BEBS as compared to Comparative Examples 1 to 7 shown in Table 3.

Note that an intermediate product (industrial product) obtained in a production process for sodium styrenesulfonate (SPINOMAR (registered trademark) NaSS manufactured by Tosoh Finechem Corporation) was used as the 2-bromoethylbenzene.

### Examples 2 to 5: Production of 4-(2-bromoethyl)benzenesulfonic acid (BEBS) (2 to 5)

Using the same raw materials as in Example 1, BEBS aqueous solutions were prepared by a similar procedure except that the compositions of initial charge and drop solutions, the drop rate and the reaction temperature were changed. As shown in Table 1, Example 2, in which the anhydrous sulfuric acid concentration in the reaction system was low, thus showed a smaller amount of nuclear-brominated BEBS as compared to Example 1, and Example 3, in which the anhydrous sulfuric acid concentration was high in the reaction system, and Example 4, in which the molar ratio of anhydrous sulfuric acid to 2-bromoethylbenzene was high, thus showed a slightly larger amount of nuclear-brominated BEBS as compared to Example 1. Example 5, in which the reaction temperature was high, nevertheless showed the same level of nuclear-brominated BEBS as in Example 3 possibly because the anhydrous sulfuric acid concentration was low. It is evident that these Examples all show a smaller content of nuclear-brominated BEBS as compared to Comparative Examples 1 to 7 shown in Table 3.

### Examples 6 to 7: Production of 4-(2-bromoethyl)benzenesulfonic acid (BEBS) (6 and 7)

BEBS aqueous solutions were synthesized by carrying out exactly the same procedure as in Examples 4 and 5 except that the 1,2-dichloroethane used in the reaction in Examples 1 to 5 was collected, then washed with water, and used as a reaction solvent.

As shown in Table 1, it is evident that Examples 6 and 7, which show a larger content of nuclear-brominated BEBS as compared to Examples 1 to 5 because the 1,2-dichloroethane contains a high amount of a moisture content, nevertheless show a smaller content of nuclear-brominated BEBS as compared to Comparative Examples 1, 2, 4, 6 and 7 in which the amount of moisture content is larger (Table 3).

### Examples 8 and 9: Production of 4-(2-bromoethyl)benzenesulfonic acid (BEBS) (8 and 9)

BEBS solutions were synthesized by carrying out exactly the same procedure as in Examples 1 to 5 except that 2-bromoethylbenzene was not washed with water and dried with molecular sieves.

As shown in Table 1, it is evident that Examples 8 and 9, which show a high content of nuclear-brominated BEBS as compared to Examples 1 to 5 because the 2-bromoethylbenzene contains a high amount of a hydrogen bromide content, nevertheless show a smaller content of nuclear-brominated BEBS as compared to Comparative Example 3 in which the amounts of hydrogen bromide and an iron content are large, and Comparative Examples 4 and 7 in which the amounts of a hydrogen bromide content, an iron content and a moisture content are large (Table 3).

Note that an intermediate product (industrial product) obtained in a production process for sodium styrenesulfonate (SPINOMAR (registered trademark) NaSS manufactured by Tosoh Finechem Corporation) was used as the 2-bromoethylbenzene.

TABLE 1
Examples 1 to 9 Example of production of BEBS

| | | Range of | Example 1 | Example 2 | Example 3 | Example 4 |
|---|---|---|---|---|---|---|
| | | claim | Effect of SO₃ concentration | | | Effect of SO₃ molar ratio |
| [Initial charge solution] | | | | | | |
| 2-BEB¹⁾ | | | 233.80 | 61.00 | 230.00 | 61.00 |
| EDC²⁾ | g | | 250.30 | 220.00 | 40.00 | 550.00 |
| [Dropped solution] | | | | | | |
| SO₃ | g | | 111.30 | 30.50 | 110.00 | 51.00 |
| Acetic acid | g | | 8.00 | 3.10 | 7.00 | 7.00 |
| EDC | g | | 250.10 | 250.00 | 220.00 | |
| [Reaction condition] | | | | | | |
| SO₃/BEB³⁾ | molar ratio | ≤2.0 | 1.10 | 1.16 | 1.11 | 1.94 |
| SO₃ concentration³⁾ | wt % | ≤20 | 12.96 | 5.37 | 18.01 | 7.58 |
| Drop time | h | 0.5~10 | 1.0 | 0.5 | 1.0 | 0.5 |
| Reaction temperature | ° C. | 10~60 | 30~40 | 30~40 | 20~30 | 20~30 |
| Aging time | h | | 1.0 | 1.0 | 1.0 | 1.0 |
| [Impurity in BEB] | | | | | | |
| Iron content | ppm | ≤5 | <1 | <1 | <1 | <1 |
| Hydrogen bromide | ppm | ≤100 | 20 | 20 | 20 | 20 |
| Moisture content | ppm | ≤1000 | 20 | 20 | 20 | 20 |
| [Impurity in EDC] | | | | | | |
| Iron content | ppm | ≤5 | <1 | <1 | <1 | <1 |
| Hydrogen bromide | ppm | ≤100 | <1 | <1 | <1 | <1 |
| Moisture content | ppm | ≤1000 | 63 | 63 | 63 | 63 |
| [After reaction] | | | | | | |
| Pure water⁴⁾ | g | | 162.8 | 48.0 | 165.0 | 50.0 |
| [Result] | | | | | | |
| BEB conversion rate | % | | 97.6 | 98.0 | 96.6 | 99.3 |
| Yield of concentrated | g | | 437.32 | 115.36 | 422.08 | 116.38 |
| BEBS⁵⁾ (aq.) | | | | | | |
| Concentration of | wt % | | 70.4 | 69.9 | 71.0 | 70.2 |
| concentrated BEBS⁶⁾ (aq.) | | | | | | |
| Purity of BEBS⁷⁾ | area % | | 96.2 | 97.1 | 95.9 | 95.7 |
| Nuclear-brominated form⁸⁾ | area % | ≤0.1 | 0.01 | ND | 0.02 | 0.05 |

| | | Example 5 | Example 6 | Example 7 | Example 8 | Example 9 |
|---|---|---|---|---|---|---|
| | | Effect of SO₃ molar ratio | Effect of moisture content | | Effect of HBr | |
| [Initial charge solution] | | | | | | |
| 2-BEB¹⁾ | | 130.00 | 233.30 | 233.80 | 235.00 | 230.00 |
| EDC²⁾ | g | 170.00 | 497.70 | 497.70 | 500.00 | 150.00 |
| [Dropped solution] | | | | | | |
| SO₃ | g | 40.00 | 111.00 | 111.60 | 111.10 | 111.10 |
| Acetic acid | g | 7.00 | 8.01 | 7.99 | 8.00 | 7.00 |
| EDC | g | | | | | 150.00 |
| [Reaction condition] | | | | | | |
| SO₃/BEB³⁾ | molar ratio | 0.71 | 1.10 | 1.11 | 1.10 | 1.11 |
| SO₃ concentration³⁾ | wt % | 11.46 | 12.98 | 13.03 | 12.93 | 16.90 |
| Drop time | h | 1.0 | 1.0 | 1.0 | 1.0 | 1.0 |
| Reaction temperature | ° C. | 50~60 | 20~30 | 30~40 | 30~40 | 30~40 |
| Aging time | h | 2.0 | 1.0 | 1.0 | 1.0 | 1.0 |
| [Impurity in BEB] | | | | | | |
| Iron content | ppm | <1 | <1 | <1 | <1 | <1 |
| Hydrogen bromide | ppm | 20 | 20 | 20 | 73 | 144 |
| Moisture content | ppm | 20 | 20 | 20 | 69 | 39 |
| [Impurity in EDC] | | | | | | |
| Iron content | ppm | <1 | <1 | <1 | <1 | <1 |
| Hydrogen bromide | ppm | <1 | <1 | <1 | <1 | <1 |
| Moisture content | ppm | 63 | 754 | 1,455 | 63 | 63 |
| [After reaction] | | | | | | |
| Pure water⁴⁾ | g | 70.0 | 162.8 | 162.8 | 162.8 | 165.0 |
| [Result] | | | | | | |
| BEB conversion rate | % | 65.0 | 96.0 | 95.5 | 96.2 | 97.3 |
| Yield of concentrated | g | 163.52 | 421.91 | 422.38 | 427.66 | 427.54 |
| BEBS⁵⁾ (aq.) | | | | | | |
| Concentration of | wt % | 69.7 | 71.6 | 71.3 | 71.3 | 70.6 |
| concentrated BEBS⁶⁾ (aq.) | | | | | | |
| Purity of BEBS⁷⁾ | area % | 96.0 | 94.2 | 93.5 | 95.0 | 94.2 |
| Nuclear-brominated form⁸⁾ | area % | 0.02 | 0.06 | 0.08 | 0.04 | 0.06 |

¹⁾2-BEB = 2-bromoethylbenzene,
²⁾EDC = 1,2-dichloroethane
³⁾Molar ratio and concentration at the end of dropping of SO³,
⁴⁾Pure water for use in extraction of BEBS from reaction solution
⁵⁾BEBS = 4-(2-bromoethyl)benzenesulfonic acid,
⁶⁾EDC and water are distilled away to adjust the concentration
⁷⁾HPLC peak area % of BEBS,
⁸⁾Peak area % of nuclear-brominated BEBS when the HPLC peak area of BEBS is defined as 100

### Example 10: Production of 4-(2-bromoethyl)benzenesulfonic acid (BEBS) (10)

A 1 L glass flask equipped with a reflux cooling tube, a nitrogen inlet tube and a thermometer insertion tube was supplied separately with a solution of 2-bromoethylbenzene in 1,2-dichloroethane (a mixed solution of 233.50 parts by weight of 2-bromoethylbenzene and 200.00 parts by weight of 1,2-dichloroethane) at a rate of 433.50 parts by weight per hour and a solution of anhydrous sulfuric acid in 1,2-dichloroethane (a mixed solution of 111.40 parts by weight of anhydrous sulfuric acid, 8.00 parts by weight of acetic acid and 300.00 parts by weight of 1,2-dichloroethane) at a rate of 419.40 parts by weight per hour, while the mixture was reacted at an internal temperature of 40 to 50° C. under stirring. The reaction liquid was intermittently extracted by a pump every 10 minutes in an amount equivalent 852.90 parts by weight per hour. Here, the apparent retention time of the reaction liquid is 1 hour, the anhydrous sulfuric acid concentration in the reactor is 12.98 wt %, and the molar ratio of anhydrous sulfuric acid to 2-bromoethylbenzene is 1.11. The reaction conversion rate of BEB was 98.2%.

Note that the 2-bromoethylbenzene and the 1,2-dichloroethane were the same as those used in Examples 1 to 5.

To 852.90 parts by weight of the extracted reaction liquid, 162.80 parts by weight of pure water was added, the mixture was sufficiently stirred, a downside aqueous solution containing BEBS was then collected. Remaining 1,2-dichloroethane and water were distilled away from the aqueous solution with a rotary evaporator to obtain 438.86 parts by weight of a concentrated BEBS aqueous solution. The BEBS concentration measured by HPLC was 70.4 wt % (the yield with respect to BEB is 93.3%).

The BEBS aqueous solution was analyzed by HPLC, and the results showed that as shown in Table 2, the purity of BEBS was 96.8 area %, and the peak area of nuclear-brominated BEBS when the peak area of BEBS is defined as 100 is 0.01%. It is thus evident that the BEBS aqueous solution has a smaller content of nuclear-brominated BEBS as compared to Comparative Examples 1 to 7 shown in Table 3.

### Example 11: Production of 4-(2-bromoethyl)benzenesulfonic acid (BEBS) (11)

A 1 L glass flask equipped with a reflux cooling tube, a nitrogen inlet tube and a thermometer insertion tube was supplied separately with 2-bromoethylbenzene at a rate of 233.50 parts by weight per hour and a solution of anhydrous sulfuric acid in 1,2-dichloroethane (a mixed solution of 111.60 parts by weight of anhydrous sulfuric acid, 8.00 parts by weight of acetic acid and 500.00 parts by weight of 1,2-dichloroethane) at a rate of 619.60 parts by weight per hour, while the mixture was reacted at an internal temperature of 40 to 50° C. under stirring. The reaction liquid was intermittently extracted by a pump every 10 minutes in an amount equivalent to 853.10 parts by weight per hour. Here, the apparent retention time of the reaction liquid is 1 hour, the anhydrous sulfuric acid concentration in the reactor is 13.00 wt %, and the molar ratio of anhydrous sulfuric acid to 2 bromethylbenzene is 1.11. The reaction conversion rate of BEB was 97.90%.

Note that the 2-bromoethylbenzene and the 1,2-dichloroethane were the same as those used in Examples 1 to 5.

To 853.10 parts by weight of the extracted reaction liquid, 163.00 parts by weight of pure water was added, the mixture was sufficiently stirred, a downside aqueous solution containing BEBS was then collected. Remaining 1,2-dichloroethane and water were distilled away from the aqueous solution with a rotary evaporator to obtain 440.65 parts by weight of a concentrated BEBS aqueous solution. The BEBS concentration measured by HPLC was 69.90 wt % (the yield with respect to BEB is 93.01%).

The BEBS aqueous solution was analyzed by HPLC, and the results showed that as shown in Table 2, the purity of BEBS was 96.3 area %, and the peak area of nuclear-brominated BEBS when the peak area of BEBS is defined as 100 is 0.01%. It is thus evident that the BEBS aqueous solution has a smaller content of nuclear-brominated BEBS as compared to Comparative Examples 1 to 7 shown in Table 3.

TABLE 2
Examples 10 to 11 Example of production of BEBS

| | | | Example | Example |
|---|---|---|---|---|
| | | | 10 | 11 |
| | | Range of | Concentration of | |
| | | claim | supplied liquid | |
| [Supplied solution A] | | | | |
| 2-BEB¹⁾ | parts by weight | | 233.50 | 233.50 |
| EDC²⁾ | parts by weight | | 200.00 | |
| Supply rate | parts by weight/h | | 433.50 | 233.50 |
| [Supplied solution B] | | | | |
| SO₃ | parts by weight | | 111.40 | 111.60 |
| Acetic acid | parts by weight | | 8.00 | 8.00 |
| EDC | parts by weight | | 300.00 | 500.00 |
| Supply rate | parts by weight/h | | 419.40 | 619.60 |
| [Reaction condition] | | | | |
| SO3/BEB³⁾ | molar ratio | 1.00~2.0 | 1.11 | 1.11 |
| SO₃ concentration³⁾ | wt % | 5~20 | 12.98 | 13.00 |
| Reaction temperature | ° C. | 10~60 | 40~50 | 40~50 |
| Reaction liquid extraction | parts by weight/h | 0.5~3 | 852.90 | 853.10 |
| rate | | | | |
| [Impurity in BEB] | | | | |
| Iron content | ppm | ≤5 | <1 | <1 |
| Hydrogen bromide | ppm | ≤100 | 20 | 20 |
| Moisture content | ppm | ≤1000 | 20 | 20 |
| [Impurity in EDC] | | | | |
| Iron content | ppm | ≤5 | <1 | <1 |
| Hydrogen bromide | ppm | ≤100 | <1 | <1 |
| Moisture content | ppm | ≤1000 | 63 | 63 |
| [After reaction] | | | | |
| Pure water²⁾ | parts by weight | | 162.8 | 163.0 |
| [Result] | | | | |
| BEB conversion rate | % | | 98.2 | 97.9 |
| Yield of concentrated | g | | 438.86 | 440.65 |
| BEBS⁵⁾ (aq.) | | | | |
| Concentration of | wt % | | 70.4 | 69.9 |
| concentrated BEBS⁶⁾ (aq.) | | | | |
| Purity of BEBS⁷⁾ | area % | | 96.8 | 96.3 |
| Nuclear-brominated | area % | ≤0.1 | 0.01 | 0.01 |
| form⁸⁾ | | | | |

¹⁾2-BEB = 2-bromoethylbenzene,
²⁾EDC = 1,2-dichloroethane
³⁾Molar ratio and concentration at the end of dropping of SO³
⁴⁾Pure water for use in extraction of BEBS from reaction solution
⁵⁾BEBS = 4-(2-bromoethyl)benzenesulfonic acid
⁶⁾EDC and water are distilled away to adjust the concentration
⁷⁾HPLC peak area % of BEBS
⁸⁾Peak area % of nuclear-brominated BEBS when the HPLC peak area of BEBS is defined as 100

### Comparative Example 1: Production of 4-(2-bromoethyl)benzenesulfonic acid (BEBS) (12)

A 1 L four-necked glass flask equipped with a reflux cooling tube, a nitrogen inlet tube, a thermometer insertion tube and a drop funnel was charged with 233.30 g (1.25 mol) of 2-bromoethylbenzene and 497.70 g of 1,2-dichloroethane. The drop funnel was charged with a mixed solution of 111.30 g (1.38 mol) of anhydrous sulfuric acid and 8.00 g (0.13 mol) of acetic acid. Note that the 2-bromoethylbenzene was washed with pure water, then treated with CHELEST FIBER (registered trademark) IRY-LC12 (manufactured by CHELEST CORPORATION) having a cation exchange property, dried with molecular sieves, and confirmed to contain an iron content at less than 1 ppm, a hydrogen bromide content at 20 ppm and a moisture content at 69 ppm in advance. Note that the 1,2-dichloroethane was obtained by collecting the 1,2-dichloroethane used in Examples 10 and 11, and performing washing with water, and contained an iron content and a hydrogen bromide content each at less than 1 ppm, and a water content at 2145 ppm.

In a nitrogen atmosphere, the mixture was sufficiently stirred with a magnetic rotor, and a mixed solution of anhydrous sulfuric acid and anhydrous acetic acid was added dropwise over 1 hour while the internal temperature was controlled to be 30 to 40° C. After the dropwise addition, the mixture was aged at 40° C. for 1 hour. The concentration of the anhydrous sulfuric acid supplied to the reactor was 0.00 to 13.01 wt % until completion of the reaction after the start of the reaction, and the molar ratio of the anhydrous sulfuric acid to the 2-bromoethylbenzene was 0.00 to 1.11. The reaction conversion rate of BEB was 96.4%.

After completion of the aging, 163.00 g of pure water was added while an internal temperature of 30 to 40° C. was maintained. The mixture was sufficiently stirred, and then left to stand, and a downside aqueous solution containing BEBS was collected using a separating funnel. Remaining 1,2-dichloroethane and water were distilled away from the aqueous solution with a rotary evaporator to obtain 420.30 g of a concentrated BEBS aqueous solution. The BEBS concentration measured by HPLC was 72.1 wt % (the yield with respect to BEB is 91.6%).

The BEBS aqueous solution was analyzed by HPLC, and the results showed that as shown in Table 3, the purity of BEBS is 91.3 area %, and the peak area of nuclear-brominated BEBS when the peak area of BEBS is defined as 100 was 0.31%. It is thus evident that the BEBS aqueous solution has a markedly high content of nuclear-brominated BEBS as compared to Examples 1 to 11 shown in Tables 1 and 2. This may be because the amount of a moisture content in the reaction system was high, and side reactions were promoted.

Note that an intermediate product (industrial product) obtained in a production process for sodium styrenesulfonate (SPINOMAR NaSS™ manufactured by Tosoh Finechem Corporation) was used as the 2-bromoethylbenzene.

### Comparative Example 2: Production of 4-(2-bromoethyl)benzenesulfonic acid (BEBS) (13)

A concentrated BEBS aqueous solution was synthesized by carrying out exactly the same procedure as in Comparative Example 1 except that a recycled product of 1,2-dichloroethane with a different amount of a moisture content was used as a reaction solvent.

As shown in Table 3, it is evident that Comparative Example 2, in which the 1,2-dichloroethane contained a still higher amount of a moisture content as compared to Comparative Example 1, thus showed an increased content of nuclear-brominated BEBS. This may be because the amount of a moisture content in the reaction system was higher, and side reactions were further promoted.

### Comparative Example 3: Production of 4-(2-bromoethyl)benzenesulfonic acid (BEBS) (14)

A concentrated BEBS aqueous solution was synthesized by carrying out exactly the same procedure as in Examples 4 and 5 except that 2-bromoethylbenzene which had not been subjected to washing with water, cation-exchange filter treatment and drying with molecular sieves was used. As shown in Table 3, it is evident that Comparative Example 3, in which the 2-bromoethylbenzene contained higher amounts of an iron content, a hydrogen bromide content and a moisture content as compared to Examples 1 to 11, thus showed a markedly increased content of nuclear-brominated BEBS. This may be because impurities in the reaction system further promoted side reactions.

Note that an intermediate product (industrial product) obtained in a production process for sodium styrenesulfonate (SPINOMAR NaSS (registered trademark) manufactured by Tosoh Finechem Corporation) was used as the 2-bromoethylbenzene.

### Comparative Example 4: Production of 4-(2-bromoethyl)benzenesulfonic acid (BEBS) (15)

A concentrated BEBS aqueous solution was synthesized by carrying out exactly the same procedure as in Comparative Example 3 except that a recycled product of 1,2-dichloroethane with a large amount of a moisture content was used. As shown in Table 3, it is evident that Comparative Example 4, which was identical in iron content and hydrogen bromide content in 2-bromoethylbenzene to Comparative Example 3, nevertheless showed a further increased content of nuclear-brominated BEBS because the amount of a moisture content increased. This may be because the multiplicative effect of impurities in the reaction system further promoted side reactions.

### Comparative Example 5: Production of 4-(2-bromoethyl)benzenesulfonic acid (BEBS) (16)

A concentrated BEBS aqueous solution was synthesized by carrying out exactly the same procedure as in Examples 4 and 5 except that the concentration of anhydrous sulfuric acid added dropwise to the reactor and the molar ratio of the anhydrous sulfuric acid to 2-bromoethylbenzene were increased. As shown in Table 3, it is evident that Comparative Example 5, which is equivalent in iron content, hydrogen bromide content and moisture content in the reaction system to Examples 1 to 11, nevertheless shows a high content of nuclear-brominated BEBS. This may be because the high concentration of anhydrous sulfuric acid promoted side reactions.

### Comparative Example 6: Production of 4-(2-bromoethyl)benzenesulfonic acid (BEBS) (17)

A concentrated BEBS aqueous solution was synthesized by carrying out exactly the same procedure as in Comparative Example 5 except that a recycled product of 1,2-dichloroethane with a large amount of a moisture content was used. As shown in Table 3, it is evident that Comparative Example 6, in which the amount of a moisture content increased, thus showed a further increased content of nuclear-brominated BEBS.

### Reference Example 1: Production of 4-(2-bromoethyl)benzenesulfonic acid (BEBS) (18)

A concentrated BEBS aqueous solution was synthesized by carrying out exactly the same procedure as in Examples 4 and 5 except that the concentration of anhydrous sulfuric acid in the reactor and the molar ratio of anhydrous sulfuric acid to 2-bromoethylbenzene were decreased. As shown in Table 3, it is evident that Reference Example 1, in which the amounts of an iron content, a hydrogen bromide content and a moisture content are low, thus shows a low content of nuclear-brominated BEBS, but is not suitable for production of an industrial raw material (precursor) of sodium 4-styrenesulfonate because a large number of raw material 2-bromoethylbenzene exists due to an extremely low reaction conversion rate of 35% although the reaction temperature is high.

### Comparative Example 7: Production of 4-(2-bromoethyl)benzenesulfonic acid (BEBS) (19)

A concentrated BEBS aqueous solution was synthesized by carrying out exactly the same procedure as in Reference Example 1 except that a recycled product of 1,2-dichloroethane with a large amount of a moisture content, and 2-bromoethylbenzene with a large amount of an iron content, a hydrogen bromide content and a moisture content as in Comparative Examples 3 and 4. It is evident that even when the concentration and the molar ratio of anhydrous sulfuric acid are low, the content of nuclear-brominated BEBS increases if the concentrations of an iron content, a hydrogen bromide content and a moisture content in the reaction system exceed a certain level. The decreased amount of anhydrous sulfuric acid leads to an extremely low reaction conversion rate of 29.7%, which is not suitable at all for practical use.

TABLE 3
Comparative Examples 1 to 7, Synthesis Example 1 Example of production of BEBS

| | | | Comparative | Comparative | Comparative | Comparative |
|---|---|---|---|---|---|---|
| | | Range of | Example 1 | Example 2 | Example 3 | Example 4 |
| | | claim | Effect of moisture content | | Effect of iron content | |
| [Initial charge solution] | | | | | | |
| 2-BEB¹⁾ | g | | 233.30 | 233.20 | 230.20 | 230.20 |
| EDC²⁾ | g | | 497.70 | 497.70 | 497.70 | 497.60 |
| [Dropped solution] | | | | | | |
| SO₃ | g | | 111.30 | 111.60 | 111.40 | 111.50 |
| Acetic acid | g | | 8.00 | 8.00 | 8.00 | 8.00 |
| EDC | g | | | | | |
| [Reaction condition] | | | | | | |
| SO₃/BEB³⁾ | molar ratio | ≤2.0 | 1.11 | 1.11 | 1.12 | 1.12 |
| SO₃ concentration³⁾ | wt % | ≤20 | 13.01 | 13.04 | 13.07 | 13.08 |
| Drop time | h | 0.5~10 | 1.0 | 2.0 | 2.0 | 1.0 |
| Reaction temperature | ° C. | 10~60 | 30~40 | 30~40 | 30~40 | 30~40 |
| Aging time | h | | 1.0 | 1.0 | 1.0 | 1.0 |
| [Impurity in BEB] | | | | | | |
| Iron content | ppm | ≤5 | <1 | <1 | 6 | 6 |
| Hydrogen bromide | ppm | ≤100 | 20 | 20 | 178 | 178 |
| Moisture content | ppm | ≤1000 | 69 | 69 | 398 | 398 |
| [Impurity in EDC] | | | | | | |
| Iron content | ppm | ≤5 | <1 | <1 | <1 | <1 |
| Hydrogen bromide | ppm | ≤100 | <1 | <1 | <1 | <1 |
| Moisture content | ppm | ≤1000 | 2,145 | 2,731 | 63 | 2,145 |
| [After reaction] | | | | | | |
| Pure water⁴⁾ | g | | 163.0 | 163.2 | 163.2 | 165.0 |
| [Result] | | | | | | |
| BEB conversion rate | % | | 96.4 | 96.2 | 96.8 | 99.3 |
| Yield of concentrated | g | | 420.30 | 426.95 | 417.02 | 426.01 |
| BEBS⁵⁾ (aq.) | | | | | | |
| Concentration of | wt % | | 72.1 | 70.8 | 72.0 | 72.3 |
| concentrated BEBS⁶⁾ (aq.) | | | | | | |
| Purity of BEBS⁷⁾ | area % | | 91.3 | 90.5 | 89.8 | 89.2 |
| Nuclear-brominated | area % | ≤0.1 | 0.31 | 0.54 | 0.91 | 2.01 |
| form⁸⁾ | | | | | | |

| | | Comparative | Comparative | Comparative | Reference |
|---|---|---|---|---|---|
| | | Example 5 | Example 6 | Example 7 | Example 1 |
| | | Effect of SO₃ amount and moisture content | | | |
| [Initial charge solution] | | | | | |
| 2-BEB¹⁾ | g | 130.00 | 130.00 | 130.00 | 130.00 |
| EDC²⁾ | g | 170.00 | 170.00 | 170.00 | 170.00 |
| [Dropped solution] | | | | | |
| SO₃ | g | 110.00 | 111.60 | 25.00 | 25.00 |
| Acetic acid | g | 7.00 | 7.99 | 1.80 | 1.80 |
| EDC | g | | | | |
| [Reaction condition] | | | | | |
| SO₃/BEB³⁾ | molar ratio | 1.96 | 1.99 | 0.45 | 0.45 |
| SO₃ concentration³⁾ | wt % | 26.22 | 26.44 | 7.60 | 7.60 |
| Drop time | h | 1.0 | 1.0 | 1.0 | 1.0 |
| Reaction temperature | ° C. | 30~40 | 30~40 | 50~60 | 50~60 |
| Aging time | h | 1.0 | 1.0 | 1.0 | 1.0 |
| [Impurity in BEB] | | | | | |
| Iron content | ppm | <1 | <1 | 6 | <1 |
| Hydrogen bromide | ppm | 20 | 20 | 178 | 20 |
| Moisture content | ppm | 20 | 20 | 398 | 20 |
| [Impurity in EDC] | | | | | |
| Iron content | ppm | <1 | <1 | <1 | <1 |
| Hydrogen bromide | ppm | <1 | <1 | <1 | <1 |
| Moisture content | ppm | 63 | 2,145 | 2,145 | 63 |
| [After reaction] | | | | | |
| Pure water⁴⁾ | g | 100.0 | 100.0 | 50.0 | 50.0 |
| [Result] | | | | | |
| BEB conversion rate | % | 99.1 | 99.4 | 29.7 | 35.0 |
| Yield of concentrated | g | 249.05 | 242.50 | 74.32 | 87.71 |
| BEBS⁵⁾ (aq.) | | | | | |
| Concentration of | wt % | 69.7 | 71.8 | 69.7 | 69.7 |
| concentrated BEBS⁶⁾ (aq.) | | | | | |
| Purity of BEBS⁷⁾ | area % | 91.0 | 90.5 | 91.2 | 96.0 |
| Nuclear-brominated | area % | 0.19 | 0.61 | 0.31 | 0.01 |
| form⁸⁾ | | | | | |

¹⁾2-BEB = 2-bromoethylbenzene,
²⁾EDC = 1,2-dichloroethane
³⁾Molar ratio and concentration at the end of dropping of SO3,
⁴⁾Pure water for use in extraction of BEBS from reaction solution
⁵⁾BEBS = 4-(2-bromoethyl)benzenesulfonic acid,
⁶⁾EDC and water are distilled away to adjust the concentration
⁷⁾HPLC peak area % of BEBS,
⁸⁾Peak area % of nuclear-brominated BEBS when the HPLC peak area of BEBS is defined as 100

### Example 12: Production of High-Purity Sodium 4-Styrenesulfonate (1)

### <Synthesis of NaSS>

A 2 L cylindrical glass separable flask equipped with a reflux cooling tube, a nitrogen inlet tube and a stirrer was charged with 276.00 g of a 12% sodium hydroxide aqueous solution and 0.80 g of sodium nitrite, and the mixture was heated to 70° C. with stirring. An internal temperature of 90° C. was maintained, and in a nitrogen atmosphere, 462.00 g of a 48% sodium hydroxide aqueous solution and 708.40 g of a 70.4 wt % BEBS aqueous solution obtained in Example 1 were each added dropwise over 3 hours with stirring. The obtained slurry of NaSS was cooled to 30° C., and separated into solid and liquid fractions with a centrifuge to obtain 310.80 g of a NaSS wet cake.

The NaSS contains impurities such as sodium bromide. Thus, in Examples and Comparative Examples below, the following purification was performed for quantifying the amount of bonded bromine.

### <Purification of NaSS>

A 1 L cylindrical glass separable flask equipped with a reflux cooling tube, a nitrogen inlet tube and a stirrer was charged with 6.16 g of sodium hydroxide, 293.00 g of pure water, 0.28 g of sodium nitride, and 308.00 g of NaSS obtained as described above, and the mixture was stirred at 60° C. for 1 hour in a nitrogen atmosphere. Thereafter, this was cooled to room temperature over 3 hours, and then subjected to solid-liquid separation with a centrifuge to obtain 272.10 g of a purified NaSS wet cake.

About 100 g of the purified NaSS cake obtained as described above was dissolved in pure water to obtain a 5 wt % aqueous solution (in terms of a net content), which was passed through a strong acid cation-exchange resin (Amberlite IR-120B manufactured by Organo Corporation, regenerated with hydrochloric acid) column and a strong base anion-exchange resin (Amberlite IRA-402BL manufactured by Organo Corporation, regenerated with sodium hydroxide) column in this order to obtain a styrenesulfonic acid aqueous solution. Since styrenesulfonic acid after cation exchange easily undergoes spontaneous polymerization, the aqueous solution discharged from the column was maintained at 5° C. or lower, and neutralized with sodium hydroxide immediately after anion exchange. The aqueous solution was concentrated with a rotary evaporator to precipitate crystals, which were separated by filtration, and dried under vacuum at 60° C. for 5 hours to obtain 64.60 g of high-purity NaSS crystals having a purity of 99.5 wt % and containing a moisture content at 0.5 wt %.

The bromine content, that is, the inorganic (non-bonding) bromine content, in the high-purity NaSS, which was quantified by ion chromatography, was at less than 1 ppm.

Organic impurities such as isomers which may be contained in the high-purity NaSS crystals were analyzed by HPLC, and the results showed that the NaSS crystals contained (a) sodium ortho-styrenesulfonate at 0.00%, (b) sodium 4-(2-bromoethyl)benzenesulfonate at 0.00%, (c) sodium meta-styrenesulfonate at 0.01%, (d) sodium bromostyrenesulfonate at 0.01% and (e) sodium 4-(2-hydroxyethyl)benzenesulfonate at 0.00% (where each value is an area ratio when the sum of HPLC peak areas of the organic impurities and NaSS is defined as 100).

From the molecular weight of NaSS which is 206.2 g/mol, the molecular weight of sodium bromostyrenesulfonate which is 285.1 g/mol, the atomic weight of Br which is 79.9 g/mol, and the HPLC area ratio (≈assumed to be a molar ratio), an approximate amount of the bromine content derived from sodium bromostyrenesulfonate contained in high-purity NaSS can be calculated as follows.

$$1 , 000 , 000 \times 79.9 \times 0 \text{.01} / ( 28 \; 5.1 \times 0.01 + 206.2 \times 99.99 ) \approx 39 ppm$$

On the other hand, the total bromine content, that is, bonded bromine, in the high-purity NaSS, which was quantified by combustion decomposition ion chromatography was 108 ppm, a value much higher than the above. In other words, a quantitation error caused by the extremely small peak of the sodium bromostyrenesulfonate, or the presence of bonded bromine other than that of sodium bromostyrenesulfonate, such as a positional isomer, was suggested. However, the total bromine amount is evidently smaller as compared to that in Comparative Examples 8 to 11 (Table 4). This may be because BEBS having a smaller content of nuclear-brominated BEBS was used as a precursor.

Further, by the following method, the high-purity NaSS was polymerized to form PSS, and a change in bromide ion concentration with time (the presence of labile bonded bromine) was examined.

### <Synthesis of Poly NaSS>

A 500 ml glass flask equipped with a reflux cooling tube, a nitrogen inlet tube and a stirrer was charged with 250.00 g of pure water, 30.02 g of the high-purity NaSS obtained as described above, and 1.00 g of a water-soluble azo-based radical polymerization initiator V-50, and the mixture was dissolved at ordinary temperature. Subsequently, the solution was deoxidized by repeating evacuation with an aspirator and introduction of nitrogen, and polymerization was then performed for 24 hours in a hot bath at 60° C. with stirring in a nitrogen atmosphere. The polymerization conversion rate of NaSS, which was determined at this time, was 100%.

Subsequently, under nitrogen flow, the stirring was continued at 60° C. for 24 hours with the solution maintained at a pH of 13 or more by adding 1.64 g of a 48 wt % sodium hydroxide aqueous solution.

The poly NaSS had a number average molecular weight Mn of 114,000 and a weight average molecular weight Mw of 285,000 (Mw/Mn=2.50) as determined by GPC.

### <Preparation of PSS and Confirmation of Stability>

The poly NaSS aqueous solution obtained as described above was treated with an ultrafiltration module (Vivaflow 200 manufactured by Sartorius AG, molecular weight cut off: 50,000), and then passed through a strong acid cation-exchange resin (Amberlite IR-120B manufactured by Organo Corporation, regenerated with hydrochloric acid) column and a strong base anion-exchange resin (Amberlite IRA-402BL manufactured by Organo Corporation, regenerated with sodium hydroxide) column in this order to obtain a PSS aqueous solution. About 1 g of the aqueous solution was precisely weighed, and dried under vacuum at 100° C. for 3 hours, the resin content was quantified, and the resin content was adjusted with pure water to obtain 230.01 g of a 10.00 wt % polystyrenesulfonic acid aqueous solution. In the PSS, the number average molecular weight was 114,000, the weight average molecular weight was 282,000 (Mw/Mn=2.47), the bromide ion concentration determined by ion chromatography was less than 1 ppm, and the sodium content quantified by ICP-AES was at less than 1 ppm. That is, free (non-bonding) bromine was sufficiently removed. On the other hand, about 10 g of the PSS aqueous solution was taken, and dried under vacuum at 110° C. for 3 hours to acquire solid PSS, and the total bromine content was analyzed by decomposition combustion ion chromatography. The result showed that the total bromine content was at 111 ppm. The total chlorine content in the solid PSS was at less than 1 ppm.

The PSS aqueous solution was divided among glass sample bottles, sealed, and aged in an oven at 70° C. A change in bromide ion concentration was monitored by ion chromatography. The results are shown in Table 4, which reveals that an increase in bromide ions with time is considerably suppressed as compared to Comparable Examples 8 to 11. This may be because the amount of bonded bromine contained in NaSS was small, that is, the amount of nuclear-brominated forms which may be contained in BEBS as a precursor was decreased.

Note that it is unlikely that chlorine remains in NaSS and a polymer thereof as long as BEBS is used as a precursor. On the other hand, if 4-(2-chloroethyl)benzenesulfonic acid (e.g., Japanese Patent Laid Open No. 1997-40633) is used as a precursor, inorganic and organic chlorine may remain as in the case of BEBS.

### Example 13: Production of High-Purity Sodium 4-Styrenesulfonate (2)

### <Synthesis of NaSS>

Under exactly the same conditions of charge weight and the like as in Example 12 except that the 69.9 wt % BEBS aqueous solution obtained in Example 2 was used, a reaction and so on are carried out to obtain 302.20 g of a NaSS wet cake.

### <Purification of NaSS>

Under exactly the same conditions as in Example 12 except that the NaSS wet cake obtained as described above was used, purification was performed to obtain 66.02 g of high-purity NaSS dry crystals. The purity was 99.5 wt %, the moisture content was at 0.5 wt %, the bromine content in the high-purity NaSS, that is, the inorganic bromine content analyzed in the form of an aqueous solution was at less than 1 ppm, and the total bromine content was at 46 ppm. It is evident that the crystals have a smaller total bromine amount as compared to Comparative Examples 8 to 11 (Table 4). This may be because BEBS having a smaller content of nuclear-brominated BEBS was used as a precursor.

Subsequently, as in Example 12, NaSS was polymerized to form PSS, and a change in bromide ion concentration with time was examined.

### <Synthesis of Poly NaSS>

Under exactly the same conditions of charge weight and the like as in Example 12 except that the high-purity NaSS crystals obtained as described above were used, NaSS was polymerized to obtain an aqueous solution of poly NaSS having a number average molecular weight Mn of 112,000 and a weight average molecular weight Mw of 281,000 (Mw/Mn=2.51).

### <Preparation of PSS and Confirmation of Stability>

Except that the poly NaSS aqueous solution obtained as described above was used, exactly the same ultrafiltration and ion-exchange treatment as in Example 12 were performed to obtain 238.96 g of a 10.00 wt % PSS aqueous solution. The number average molecular weight was 112,000, the weight average molecular weight was 281,000 (Mw/Mn=2.51), the bromide ion concentration was less than 1 ppm, and the sodium content was at less than 1 ppm. As in Example 12, solid PSS was acquired, and the total bromine content was analyzed. The result showed that the total bromine content was at 47 ppm, a value slightly smaller than that in Example 12. Note that the total chlorine content in the solid PSS was at less than 1 ppm.

Subsequently, as in Example 12, the PSS aqueous solution was aged, and a change in bromide ion concentration was monitored. The results are shown in Table 4, which reveals that an increase in bromide ions with time is considerably suppressed as compared to Comparable Examples 8 to 11. This may be because the amount of bonded bromine contained in NaSS was small, that is, the amount of nuclear-brominated forms which may be contained in BEBS as a precursor was decreased.

### Example 14: Production of High-Purity Sodium 4-Styrenesulfonate (3)

### <Synthesis of NaSS>

Under exactly the same conditions of charge weight and the like as in Example 12 except that the 71.3 wt % BEBS aqueous solution obtained in Example 7 was used, a reaction and so on were carried out to obtain 316.10 g of a NaSS wet cake.

### <Purification of NaSS>

Under exactly the same conditions of charge weight and the like as in Example 12 except that the NaSS obtained as described above was used, purification was performed to obtain 63.60 g of high-purity NaSS dry crystals. The purity was 99.5 wt %, the moisture content was at 0.5 wt %, the bromine content in the high-purity NaSS, that is, the inorganic bromine content analyzed in the form of an aqueous solution was at less than 1 ppm, and the total bromine content was at 302 ppm. Since BEBS having a large amount of nuclear-brominated BEBS content was used, the crystals have a larger amount of a total bromine content as compared to Examples 12 and 13, which nevertheless, is evidently smaller than that in Comparative Examples 8 to 11.

Subsequently, as in Example 12, NaSS was polymerized to form PSS, and a change in bromide ion concentration (the presence of labile bonded bromine) with time was examined.

### <Synthesis of Poly NaSS>

Under exactly the same conditions of charge weight and the like as in Example 12 except that the high-purity NaSS crystals obtained as described above were used, NaSS was polymerized to obtain an aqueous solution of poly NaSS having a number average molecular weight Mn of 113,000 and a weight average molecular weight Mw of 283,000 (Mw/Mn=2.50).

### <Preparation of PSS and Confirmation of Stability>

Except that the poly NaSS aqueous solution obtained as described above was used, exactly the same ultrafiltration and ion-exchange treatment as in Example 12 were performed to obtain 241.95 g of a 10.00 wt % PSS aqueous solution. The number average molecular weight was 113,000, the weight average molecular weight was 283,000 (Mw/Mn=2.50), the bromide ion concentration was less than 1 ppm, and the sodium content was at less than 1 ppm. As in Example 12, solid PSS was acquired, and the total bromine content was analyzed. The result showed that the total bromine content was at 315 ppm, and the total chlorine content was at less than 1 ppm.

Subsequently, as in Example 12, the PSS aqueous solution was aged, and a change in bromide ion concentration was monitored. The results are shown in Table 4, which reveals that an increase in bromide ions with time is considerably suppressed as compared to Comparable Examples 8 to 11. This may be because the amount of bonded bromine contained in NaSS was small, that is, the amount of nuclear-brominated forms which may be contained in BEBS as a precursor was decreased.

### Example 15: Production of Ethyl Styrenesulfonate (ETSS) (1)

<Synthesize of 4-styrenesulfonyl chloride (CISS)>

A 3 L four-necked glass flask equipped with a reflux cooling tube, a nitrogen inlet tube and a stirrer was charged with 300.00 g (1.45 mol) of the high-purity NaSS crystals obtained under the conditions of Example 12, 600.00 g of toluene, 106.00 g (1.44 mol) of N,N-dimethylformamide and 0.12 g (0.1 mmol) of an antioxidant Irganox (registered trademark) 1010, and the mixture was stirred for 30 minutes in a nitrogen atmosphere while an internal temperature of 0° C. was maintained. Subsequently, while control was performed so that the internal temperature did not exceed 5° C., 236.0 g (1.94 mol) of thionyl chloride was added dropwise over 2 hours, and the stirring was continued for 3 hours. Next, while control was performed so that the internal temperature did not exceed 20° C., 750.00 g of pure water was added, and the mixture was sufficiently stirred, and then left to stand to allow separation into two layers, out of which the aqueous layer was discarded. To the organic layer left, 750.00 g of a 20 wt % sodium chloride aqueous solution was added, and the mixture was sufficiently stirred, and then left to stand to allow separation into two layers, out of which the aqueous layer was discarded. Thereafter, control was performed so that the internal temperature did not exceed 10° C., and nitrogen was fed into the reaction liquid with stirring for 12 hours to remove components derived from thionyl chloride. Thereafter, 750.00 g of pure water was added to the organic layer again, and washing of the organic layer with water was repeated until the aqueous layer had an electric conductivity of 1 μS/cm or less, thereby obtaining 760.00 g of a CISS solution. The CISS concentration determined by ¹H-NMR was 36.2 wt %. That is, the net content of CISS was 275.10 g (1.36 mol), and the yield with respect to charge NaSS was 94%.

### <Synthesis of ETSS>

A 200 ml four-necked glass flask equipped with a reflux cooling tube, a nitrogen inlet tube and a stirrer was charged with 38.5 g (0.069 mol) of the CISS solution obtained as described above, 9.2 g (0.200 mol) of ethanol and 6 mg (0.005 mmol) of an antioxidant Irganox (registered trademark) 1010, and the internal temperature was controlled so as not to exceed 0° C. Thereto, an aqueous solution composed of 15.4 g (0.132 mol) of a 48 wt % potassium hydroxide aqueous solution and 9.2 g of pure water was added dropwise over 5 hours, and the mixture was aged for 5 hours to perform esterification. Meanwhile, control was performed so that the internal temperature did not exceed 20° C. Subsequently, 100.00 g of pure water was added, the mixture was stirred, and then left to stand, and the aqueous layer containing potassium chloride was discarded, followed by washing with a 20 wt % sodium chloride solution. Further, pure water was added, the mixture was washed until the aqueous layer had an electric conductivity of 1 μS/cm or less, and the organic layer was collected. Toluene was distilled away under reduced pressure at 40° C. with a rotary evaporator to obtain 12.01 g of ETSS. The purity on an area % basis which was determined by gas chromatography was 94.00% (the major impurity was toluene contained in the CISS solution), and the yield with respect to CISS was 77%.

The bromine content in the ETSS which was quantified by ion chromatography, that is, the inorganic bromine content extracted with pure water, is at less than 1 ppm, and the total bromine content quantified by combustion decomposition ion chromatography is at 84 ppm. It is thus evident that the amount of a bromine content is smaller as compared to that in Comparative Examples 12 to 14 (Table 4). This may be because NaSS derived from BEBS having a low content of nuclear-brominated BEBS was used as a raw material.

Subsequently, by the following method, the ETSS was polymerized to form PSS, and a change in bromide ion concentration with time (the presence of labile bonded bromine) was examined.

### <Synthesis of Poly ETSS>

A 300 ml four-necked glass flask equipped with a reflux cooling tube, a nitrogen inlet tube and a stirrer was charged with 10.75 g (46.80 mmol) of ETSS obtained as described above, 40.00 g of anisole, 0.15 g (0.94 mmol) of 2,2,6,6-tetramethyl-1-piperidinyloxy (TEMPO) and 0.40 g (2.39 mmol) of azobis-isobutyronitrile, the mixture was deoxidized by repeating decompression and introduction of nitrogen with stirring with a magnetic rotor, and polymerization was then performed at 110° C. for 20 hours and then at 135° C. for 2 hours in a nitrogen atmosphere to prepare poly ETSS.

### <Preparation of PSS and Confirmation of Stability>

The reaction liquid was allowed to cool to 100° C., 75.00 g (93.75 mmol) of a 5 wt % sodium hydroxide was then added, the mixture was stirred for 5 hours, the insoluble content was separated by filtration, and the aqueous layer containing poly NaSS was collected by a liquid separation operation. The aqueous solution was slowly added dropwise to 2 L of acetone with vigorous stirring, and the precipitated polymer was dried under vacuum at 110° C. for 10 hours to collect 3.67 g of poly NaSS (yield with respect to ETSS: 38%). The poly NaSS had a number average molecular weight of 9,000 and a weight average molecular weight of 11,000 (Mw/Mn=1.22). The poly NaSS was dissolved in pure water, the solution was treated with an ultrafiltration module (Vivaflow 200 manufactured by Sartorius AG, molecular weight cut off: 5,000), and ion-exchange treatment was then performed as in Example 12 to obtain 26.77 g of a 10.00 wt % PSS aqueous solution. The number average molecular weight was 9,000, the weight average molecular weight was 11,000 (Mw/Mn=1.22), and the bromide ion concentration was less than 1 ppm, and the sodium content was at less than 1 ppm. Thus, it can be said that free bromine which is not bonded to the polymer was sufficiently removed by the ion-exchange treatment.

As in Example 12, the PSS was aged, and a change in bromide ion concentration was monitored. The results are shown in Table 4, which reveals that an increase in bromide ions with time is considerably suppressed as compared to Comparable Examples 12 to 14. This may be because the amount of bonded bromine contained in ETSS was small, that is, the amount of nuclear-brominated forms which may be contained in BEBS as a precursor was decreased.

### Example 16: Production of Ethyl Styrenesulfonate (ETSS) (2)

### <Synthesis of CISS>

Under exactly the same conditions as in Example 15 except that the high-purity NaSS crystals obtained in Example 13 were used, and the scale was reduced to one tenth, 75.00 g of a CISS solution was obtained. The CISS concentration determined by ¹H-NMR was 35.9 wt %. That is, the amount of the net content was 26.93 g, and the yield with respect to charge NaSS was 92%.

### <Synthesis of ETSS>

Under exactly the same conditions as in Example 15 except that the CISS obtained as described above was used, ETSS was synthesized to obtain 11.90 g of ETSS. The purity on an area % basis which was determined by gas chromatography was 95.00% (the major impurity is toluene contained in the CISS solution), and the yield with respect to CISS was 78%.

The bromine content in the ETSS which was quantified by ion chromatography, that is, the inorganic bromine content extracted with pure water, is at less than 1 ppm, and the total bromine content quantified by combustion decomposition ion chromatography is at 51 ppm. It is thus evident that the amount of a bromine content is smaller as compared to that in Comparative Examples 12 to 14 (Table 4). This may be because NaSS derived from BEBS having a low content of nuclear-brominated BEBS was used as a raw material.

Subsequently, as in Example 15, ETSS was polymerized to form PSS, and a change in bromide ion concentration with time was examined.

### <Synthesis of Poly ETSS>

Under exactly the same conditions as in Example 15 except that the ETSS obtained as described above was used, ETSS was polymerized to prepare poly ETSS.

### <Preparation of PSS and Confirmation of Stability>

Except that the poly ETSS obtained as described above was used, exactly the same procedure as in Example 15 was carried out to obtain 26.98 g of a 10.00 wt % PSS aqueous solution. The number average molecular weight was 9,000, the weight average molecular weight was 11,000 (Mw/Mn=1.22), the bromide ion concentration was less than 1 ppm, and the sodium content was at less than 1 ppm.

As in Example 12, the PSS aqueous solution was aged, and a change in bromide ion concentration was monitored. The results are shown in Table 4, which reveals that an increase in bromide ions with time is considerably suppressed as compared to Comparable Examples 12 to 14. This may be because the amount of bonded bromine contained in ETSS was small, that is, the amount of nuclear-brominated forms which may be contained in BEBS as a precursor was decreased.

### Example 17: Production of Ethyl Styrenesulfonate (ETSS) (3)

### <Synthesis of CISS>

Under exactly the same conditions of charge weight and the like as in Example 16 except that the high-purity NaSS crystals obtained in Example 14 were used, a reaction and the like were carried out to obtain 76.30 g of a CISS solution. The CISS concentration determined by ¹H-NMR was 35.50 wt %. That is, the amount of the net content of CISS was 27.09 g, and the yield with respect to charge NaSS was 92%.

### <Synthesis of ETSS>

Under exactly the same conditions as in Example 16 except that the CISS obtained as described above was used, a reaction and the like were carried out to obtain 12.10 g of ETSS. The purity on an area % basis which was determined by gas chromatography was 95.00% (the major impurity is toluene contained in the CISS solution), and the yield with respect to CISS was 80%.

The bromine content in the ETSS which was quantified by ion chromatography, that is, the inorganic bromine content extracted with pure water, is at less than 1 ppm, and the total bromine content quantified by combustion decomposition ion chromatography is at 338 ppm. It is thus evident that the amount of a bromine content is smaller as compared to that in Comparative Examples 12 to 14 (Table 4). However, since BEBS having a large amount of a nuclear-brominated BEBS content was used as the high-purity NaSS raw material, the ETSS had a larger amount of a total bromine content as compared to Examples 15 and 16.

Subsequently, as in Example 15, ETSS was polymerized to form PSS, and a change in bromide ion concentration with time was examined.

### <Synthesis of Poly ETSS>

By exactly the same procedure as in Example 16 except that the ETSS obtained as described above was used, ETSS was polymerized to prepare poly ETSS.

### <Preparation of PSS and Confirmation of Stability>

By exactly the same procedure as in Example 16 except that the poly ETSS obtained as described above was used, 26.65 g of a 10.00 wt % PSS aqueous solution was obtained. The number average molecular weight was 9,000, the weight average molecular weight was 11,000 (Mw/Mn=1.22), the bromide ion concentration was less than 1 ppm, and the sodium content was at less than 1 ppm.

As in Example 15, the PSS aqueous solution was aged, and a change in bromide ion concentration was monitored. The results are shown in Table 4, which reveals that an increase in bromide ions with time is considerably suppressed as compared to Comparable Examples 12 to 14. This may be because the amount of bonded bromine contained in ETSS was small, that is, the amount of nuclear-brominated forms which may be contained in BEBS as a precursor was decreased.

### Example 18: Production of High-Purity Neopentyl Styrenesulfonate (NPSS) (1)

### <Synthesis of NPSS>

A 1 L four-necked glass flask equipped with a reflux cooling tube, a nitrogen inlet tube and a stirrer was charged with 54.00 g (0.60 mol) of neopentyl alcohol and 171.3 g (2.14 mol) of pyridine. While an internal temperature of 0° C. was maintained, the mixture was stirred with a magnetic rotor, thereby being dissolved. While control was performed so that the internal temperature did not exceed 0° C., 323.21 g (0.58 mol) of the 36.20 wt % CISS solution obtained in Example 15 was added dropwise to a reactor over 2.5 hours to carry out a reaction. Excess pyridine and toluene were distilled away with an evaporator, the contents were then put in 1 L of hexane, and the mixture was cooled to −15° C. to collect white crystals. The white crystals were purified by recrystallization using a mixed solvent of hexane and toluene at 1:1, thereby obtaining 37.5 g of white crystals of NPSS. The yield with respect to CISS was 25%, and the purity determined by ¹H-NMR (internal standard substance: 1,3,5-trimethylbenzene) was 97.5%. The bromine content in the NPSS which was quantified by ion chromatography, that is, the inorganic bromine content extracted with pure water, is at less than 1 ppm, and the total bromine content quantified by combustion decomposition ion chromatography is at 122 ppm. It is thus evident that the amount of a bromine content is smaller as compared to that in Comparative Example 15 (Table 4). This may be because NaSS derived from BEBS having a low content of nuclear-brominated BEBS was used as a raw material.

Further, by the following method, the NPSS was polymerized to form PSS, and a change in bromide ion concentration with time (the presence of labile bonded bromine) was examined.

### <Synthesis of Poly NPSS>

A 200 ml four-necked glass flask equipped with a reflux cooling tube, a nitrogen inlet tube and a stirrer was charged with 10.00 g (39.32 mmol) of the NPSS obtained, 40.00 g of N,N-dimethylformamide and 325 mg (1.98 mmol) of azobis-isobutyronitrile, the mixture was deoxidized by repeating decompression and introduction of nitrogen with stirring, and polymerization was then performed at 70° C. for 25 hours in a nitrogen atmosphere. The polymerization solution was slowly added dropwise to 1 L of hexane with vigorous stirring to isolate poly NPSS. The polymer was dissolved in chloroform again, and the solution was added dropwise to hexane as a poor solvent to purify the polymer. The wet polymer was dried under vacuum at 90° C. for 10 hours, and 7.10 g of poly NPSS was collected (yield with respect to NPSS: 71%). The polymer had a number average molecular weight Mn of 18,000 and a weight average molecular weight Mw of 45,000 (Mw/Mn=2.50) as measured by GPC.

### <Synthesis of PSS and Confirmation of Stability>

7.10 g of the poly NPSS obtained as described above was dissolved in 60.0 g of dichloromethane, 14.76 g of trimethyl iodide (2.5 equivalents with respect to neopentyl sulfonate groups) was then added, and the mixture was stirred at room temperature for 4 hours. Subsequently, the dichloromethane was distilled away under reduced pressure to collect a polymer, the polymer was put in a mixed solution composed of 40 ml of 1 N hydrochloric acid and 40 ml of methanol, the mixture was stirred at room temperature for 2 hours, and the solvent was then distilled away under reduced pressure to obtain PSS.

The PSS was dissolved in ion-exchange water, the solution was treated with an ultrafiltration module (Vivaflow 200 manufactured by Sartorius AG, molecular weight cut off: 10,000), and ion-exchange treatment was then performed as in Example 15 to obtain 56.80 g of a 10.00 wt % PSS aqueous solution. The number average molecular weight was 18,000, the weight average molecular weight was 45,000 (Mw/Mn=2.50), and the bromide ion concentration was less than 1 ppm, and the sodium content was at less than 1 ppm. Thus, it can be said that free bromine compound which is not bonded to the polymer was sufficiently removed by the purification treatment.

As in Example 12, the PSS aqueous solution was aged, and a change in bromide ion concentration was monitored. The results are shown in Table 4, which reveals that an increase in bromide ions with time is considerably suppressed as compared to Comparable Example 15. This may be because the amount of bonded bromine contained in NPSS was small, that is, the amount of nuclear-brominated forms which may be contained in BEBS as a precursor was decreased.

### Example 19: Production of High-Purity 4-Styrenesulfonyl(Trifluoromethylsulfonylimide) Sodium (TfNS-Na) (1)

### <Synthesis of TfNS-Na>

A 500 ml four-necked glass flask equipped with a reflux cooling tube, a nitrogen inlet tube and a stirrer was charged with 14.92 g (98.07 mmol) of trifluoromethanesulfonamide, 116.00 g of ethyl acetate, 0.62 g (4.97 mmol) of 4-dimethylaminopyridine and 1.02 g of tert-butylcatechol, the mixture was stirred at ordinary temperature and dissolved, and 21.24 g (198.39 mmol) of sodium carbonate was then added. The internal temperature was raised to 50° C., and 54.88 g (98.03 mmol) of the 36.2 wt % CISS solution obtained in Example 15 was then added dropwise over 1 hour while an internal temperature of 50 to 60° C. was maintained. Further, the mixture was aged at 60° C. for 4 hours, and then cooled to 35° C., 90.00 g of ion-exchange water was added, the mixture was vigorously stirred, and left to stand, followed by liquid separation, and the aqueous layer containing sodium chloride was discarded. Further, 50.00 g of a 20 wt % sodium chloride aqueous solution was added, the mixture was vigorously stirred, and left to stand, followed by liquid separation, and the aqueous layer was discarded. To the organic layer, 60.00 g of toluene which is a poor solvent was added to obtain a homogeneous solution, and ethyl acetate which is a good solvent was then distilled away under reduced pressure. The precipitated crystals were separated by filtration, and dried under vacuum at room temperature for 24 hours to obtain 25.34 g of TfNS-Na (yield 73%).

### <Purification of TfNS-Na>

The TfNS-Na obtained as described above was dissolved in ion-exchange water to obtain a 5 wt % aqueous solution. Cation-exchange and anion-exchange treatments were performed as in Example 12 while care is taken to ensure that the temperature of the aqueous solution did not exceed 10° C., thereby obtaining a TfNS-H aqueous solution. Since TfNS-H after cation exchange easily undergoes spontaneous polymerization, the aqueous solution discharged from the column was maintained at 5° C. or lower, and neutralized with sodium hydroxide immediately after anion exchange.

Water was distilled away from the aqueous solution with a rotary evaporator to precipitate crystals, which were separated by filtration, and dried under vacuum at 60° C. for 5 hours to obtain 21.30 g of high-purity TfNS-Na. The purity was 99.5 wt % and the moisture content was at 0.5 wt % when determined by ¹H-NMR (internal standard substance: 1,3,5-trimethylbenzene), the bromine content quantified by ion chromatography, that is, the inorganic bromine content analyzed in the form of an aqueous solution was at less than 1 ppm, and the total bromine quantified by combustion decomposition ion chromatography was at 101 ppm. It is thus evident that the amount of total bromine is smaller as compared to that in Comparative Example 16 (Table 4). This may be because sodium styrenesulfonate derived from BEBS having a low content of nuclear-brominated BEBS was used as a raw material.

Further, by the following method, the TENS-Na was polymerized, and a change in bromide ion concentration with time (the presence of labile bonded bromine) was examined.

### <Synthesis of Poly TfNS>

A monomer aqueous solution obtained by dissolving 20.00 g (58.71 mmol) of the TfNS-Na obtained as described above in 90.00 g of ion-exchange water and a radical polymerization initiator aqueous solution obtained by dissolving 0.10 g (0.44 mmol) in ammonium persulfate in 10.00 g of ion-exchange water were each deoxidized by repeating a procedure in which

decompression with an aspirator was performed, followed by introduction of nitrogen. Polymerization was performed at the bath temperature of 85° C. with the aqueous solutions simultaneously added dropwise in 3 hours to a 200 ml four-necked glass flask equipped with a reflux cooling tube, a nitrogen inlet tube and a stirrer. Thereafter, the polymerized product was aged at 85° C. for 2 hours. The polymerization conversion rate was 98.7%, the number average molecular weight was 35,000 and the weight average molecular weight was 82,000 (Mw/Mn=2.34) when measured by GPC.

The poly TfNS-Na aqueous solution was subjected to ultrafiltration and ion-exchange treatment as in Example 18 to obtain 146.88 g of a 10.00 wt % poly TfNS-H aqueous solution. The bromide ion concentration determined by ion chromatography was less than 1 ppm, and the sodium content quantified by the ICP-AES method was at less than 1 ppm.

### <Stability of Poly TfNS-H>

A change in bromide ion concentration was monitored by ion chromatography as in Example 12. The results are shown in Table 4, which reveals that an increase in bromide ions with time is considerably suppressed as compared to Comparative Example 16. This may be because the amount of bonded bromine in TINS-Na was small, that is, the amount of nuclear-brominated forms which may be contained in BEBS as a precursor was decreased.

### Example 20: Production of High-Purity Lithium 4-Styrenesulfonate (LiSS) (1)

### <Synthesis of LiSS>

A 1 L cylindrical glass separable flask equipped with a reflux cooling tube, a nitrogen inlet tube and a stirrer was charged with 130.60 g (3.23 mol) of lithium hydroxide monohydrate, 0.60 g (0.009 mol) of sodium nitrite and 351.00 g of ion-exchange water, and the mixture was heated to 70° C. with stirring. An internal temperature of 90° C. was maintained, 457.75 g (1.21 mol) of the 70.20 wt % BEBS aqueous solution obtained in Example 4 was added over 1 hour with stirring in a nitrogen atmosphere, and the mixture was aged for 0.5 hours. The reaction solution was cooled to 20° C. over 3 hours, and then aged for 2 hours, and the obtained slurry of LiSS was subjected to solid-liquid separation with a centrifuge to obtain 199.70 g (0.89 mol) of a LiSS wet cake having a purity of 85.0%.

### <Purification of LiSS>

The wet cake and 1 L of acetone were taken in a 2 L glass beaker, and stirred at ordinary temperature for 1 hour, and wet LiSS was then collected using a Buchner funnel. The wet LiSS was put in 1 L acetone again, the mixture was stirred at ordinary temperature for 1 hour, and wet LiSS was collected using a Buscher funnel, and dried under vacuum in an oven at 60° C. for 10 hours to obtain 73.50 g of high-purity LiSS. The purity was 98.7 wt %, the moisture content was at 1.30 wt %, the bromine content in the high-purity LiSS which was quantified by ion chromatography, that is, the inorganic bromine content analyzed in the form of an aqueous solution was at less than 1 ppm, and the total bromine content quantified by combustion decomposition ion chromatography was 296 ppm. It is thus evident that the amount of the total bromine content is smaller as compared to that in Comparative Example 17 (Table 4). This may be because BEBS having a low content of nuclear-brominated BEBS was used as a raw material.

Further, by the following method, the LiSS was polymerized to form PSS, and a change in bromide ion concentration with time (the presence of labile bonded bromine) was examined.

### <Synthesis of Poly LiSS>

A 500 ml glass flask equipped with a reflux cooling tube, a nitrogen inlet tube and a stirrer was charged with 250.00 g of pure water, 30.00 g (0.16 mol) of the high-purity LiSS obtained as described above and 2.10 g (0.008 mol) of a water-soluble azo-based radical polymerization initiator V-50, and the mixture was dissolved at ordinary temperature. Subsequently, the solution was deoxidized by repeating evacuation with an aspirator and introduction of nitrogen, and polymerization was then performed for 24 hours in a hot bath at 60° C. with stirring in a nitrogen atmosphere. At this time, the polymerization conversion rate of LiSS was 100%.

Subsequently, under nitrogen flow, the stirring was continued at 60° C. for 24 hours with the solution maintained at a pH of 13 or more by adding 1.70 g of a 40 wt % lithium hydroxide aqueous solution, thereby obtaining a poly LiSS aqueous solution. The number average molecular weight Mn was 65,000 and the weight average molecular weight Mw was 161,000 (Mw/Mn=2.48).

### <Preparation of PSS and Confirmation of Stability>

The poly LiSS aqueous solution obtained as described above was subjected to ultrafiltration and ion-exchange treatment under the same conditions as in Example 18 to obtain 236.88 g of a 10.00 wt % PSS aqueous solution. The bromide ion concentration was less than 1 ppm, and the sodium content was at less than 1 ppm.

As in Example 12, a change in bromide ion concentration was monitored by ion chromatography. The results are shown in Table 4, which reveals that an increase in bromide ions with time is considerably suppressed as compared to Comparable Example 17. This may be because the amount of bonded bromine contained in LiSS was small, that is, the amount of nuclear-brominated forms which may be contained in BEBS as a precursor was decreased.

### Production of Sodium Styrenesulfonate/Styrene Copolymer (ST-3510)

<Synthesis of NaSS/styrene copolymer>

A 500 ml three-necked flask equipped with a reflux cooling tube, a nitrogen inlet tube and a stirrer was charged with 26.60 g of the unpurified NaSS obtained in Example 12 (purity: 88.5%, 114.17 mmol), 121.00 g of ion-exchange water, 69.89 g of 2-propanol, 6.37 g (60.55 mmol) of styrene and 0.92 g (3.36 mmol) of a water-soluble azo-based radical polymerization initiator V-50, the mixture was dissolved, and the solution was deoxidized by repeating degassing under reduced pressure and introduction of nitrogen. Thereafter, the flask was immersed in a hot bath at 60° C., followed by polymerization for 25 hours with stirring. The polymerization conversion rate of the sodium styrenesulfonate was 100%, the polymerization conversion rate of the styrene was 98%, the number average molecular weight was 33,000, the weight average molecular weight was 74,000 (Mw/Mn=2.24), and the copolymer composition calculated from the polymerization conversion rates was NaSS/St=66/34 on a molar ratio basis. The isopropanol and water were distilled away under reduced pressure to obtain a 15 wt % polymer aqueous solution.

### <Synthesis of Styrenesulfonic Acid/Styrene Copolymer>

The NaSS/styrene copolymer was subjected to ultrafiltration and ion-exchange treatment in the same conditions as in Example 19 to obtain 231.28 g of a 10.0 wt % aqueous solution of a styrenesulfonic acid/styrene copolymer. The bromide ion concentration was less than 1 ppm, and the sodium content was at less than 1 ppm.

### <Stability of Styrenesulfonic Acid/Styrene Copolymer>

As in Example 12, the styrenesulfonic acid/styrene copolymer aqueous solution obtained as described above was aged, and a change in bromide ion concentration was monitored. The results are shown in Table 4, which reveals that stability equivalent to that in Example 12 is exhibited. This may be because the amount of bonded bromine contained in NaSS was small, that is, the amount of nuclear-brominated forms which may be contained in BEBS as a precursor was decreased.

### Example 22: Production of Sodium Styrenesulfonate/Methacrylic Acid Copolymer

### <Synthesis of NaSS/Methacrylic Acid Copolymer>

A 500 ml three-necked flask equipped with a reflux cooling tube, a nitrogen inlet tube and a stirrer was charged with 35.00 g of the unpurified NaSS obtained in Example 12 (purity: 88.5%, 150.22 mmol), 250.00 g of ion-exchange water, 3.35 g (38.52 mmol) of methacrylic acid and 2.50 g (9.13 mmol) of a water-soluble azo-based radical polymerization initiator V-50, the mixture was dissolved, and the solution was deoxidized by repeating degassing under reduced pressure and introduction of nitrogen. Thereafter, the flask was immersed in a hot bath at 60° C., followed by polymerization for 25 hours with stirring. The polymerization conversion rate of NaSS was 100%, the polymerization conversion rate of the methacrylic acid was 96%, the number average molecular weight was 46,000, the weight average molecular weight was 129,000 (Mw/Mn=2.80), and the copolymer composition calculated from the polymerization conversion rates was NaSS/MAA=80/20 on a molar ratio basis. Water was distilled away under reduced pressure with a rotary evaporator to obtain a 15 wt % polymer aqueous solution.

### <Synthesis of Styrenesulfonic Acid/Methacrylic Acid Copolymer>

The NaSS/methacrylic acid copolymer was subjected to ultrafiltration and ion-exchange treatment under the same conditions as in Example 19 to obtain 262.26 g of a 10.0 wt % aqueous solution of a styrenesulfonic acid/methacrylic acid copolymer. The bromide ion concentration was less than 1 ppm, and the sodium content was at less than 1 ppm.

### <Stability of Styrenesulfonic Acid/Methacrylic Acid Copolymer>

As in Example 12, the styrenesulfonic acid/methacrylic acid copolymer aqueous solution was aged, and a change in bromide ion concentration was monitored. The results are shown in Table 4, which reveals that stability equivalent to that in Example 12 is exhibited. This may be because the amount of bonded bromine contained in NaSS was small, that is, the amount of nuclear-brominated forms which may be contained in BEBS as a precursor was decreased.

### Example 23: Production of lithium bis-(4-styrenesulfonyl)imide (BVBSI-Li) (1)

### <Synthesis of 4-Styrenesulfonamide>

A 300 mL glass flask reactor equipped with a reflux cooling tube, a nitrogen inlet tube and a drop tube was charged with 30.0 g (54.20 mmol) of the CISS solution synthesized in Example 15 and 30.00 g of tetrahydrofuran, and the mixture was stirred and dissolve at room temperature. The solution was cooled to 0° C., and 30.00 g (493.25 mmol) of a 28% ammonia aqueous solution (manufactured by Kishida Chemical Co., Ltd.) was added dropwise over 1 hour. After completion of the dropwise addition, the mixture was stirred at room temperature for 2 hours. After completion of the reaction, 30.00 g of ion-exchange water and 30.00 g of ethyl acetate were added, and a liquid separation operation was carried out to obtain an organic layer containing 4-styrenesulfonamide. The organic layer was concentrated to obtain 6.05 g of a white solid of 4-styrenesulfonamide (yield 65%).

### <Synthesis of BVBSI-Li>

A 100 ml glass flask reactor equipped with a reflux cooling tube, a nitrogen inlet tube and a drop tube was charged with 5.00 g (28.96 mmol) of the 4-styrenesulfonamide obtained as described above, 0.55 g (65.72 mmol) of lithium hydride and 30.00 g of dehydrated tetrahydrofuran, and the mixture was stirred at room temperature in a nitrogen atmosphere.

Next, 16.00 g (28.59 mmol) of the CISS solution synthesized in Example 15 was added dropwise to the slurry solution at ordinary temperature. After completion of the dropwise addition, the mixture was heated to 60° C., and stirred for 3 hours. The solvent was distilled away with a rotary evaporator to collect a white solid. The white solid was washed with diethyl ether, and then recrystallized using methanol to obtain 6.74 g of white crystals of BVBSI-Li. The yield with respect to CISS was 64%, and the purity determined by ¹H-NMR (internal standard substance: 1,3,5-trimethylbenzene) was 94.0%. The bromine content in the high-purity BVBSI-Li which was quantified by ion chromatography, that is, the inorganic bromine content analyzed in the form of an aqueous solution was at less than 1 ppm, and the total bromine content quantified by combustion decomposition ion chromatography was at 121 ppm.

### Synthesis Example 2: Production of Lithium Polystyrenesulfonate Crosslinked Product

2.00 g (10.21 mmol) of the LiSS obtained in Example 20, 0.50 g (1.06 mmol) of BVBSI-Li obtained in Example 23, and 0.005 g (0.018 mmol) of a water-soluble azo-based polymerization initiator V-50 were dissolved in 3.50 g of ion-exchange water to prepare a mixed monomer solution. The monomer solution was added dropwise to a transparent glass plate (thickness 1.5 mm, 5 cm×5 cm square) on which a PET film spacer (a 0.5 mm-thick film of 5 cm×5 cm square with a hole of 3 cm×3 cm in the center) was placed, and the same transparent glass plate was then superposed thereon to eliminate an excess monomer solution. The two glass plates were fixed with a metal clip, and then irradiated with 365 nm wavelength light from a distance of 5 cm in a direction perpendicular to the glass surface for 3.0 hours. Note that the illuminance at a position of 5 cm from the LED-irradiated surface in the perpendicular direction was 100 mW/cm². The metal clip was removed, and the glass plates were placed in a 1 L plastic beaker filled with ion-exchange water, and were ultrasonicated at ordinary temperature for 10 minutes with the beaker placed in an ultrasonic washing machine. As a result, the glass plates were detached to obtain a swollen sheet-shaped crosslinked product.

Thus, by using LiSS and BVBSI-Li having a decreased amount of bonded bromine, an electrolyte film and a coating film in which release of bromine over time is suppressed can be easily formed.

TABLE 4
Example of production of styrenesulfonic acid compound

| | | | | BEBS property | | Monomer property | |
|---|---|---|---|---|---|---|---|
| | | | | | Nuclear- | | Total |
| | | Raw | Raw | | brominated | | bonded |
| | Monomer | material | material | | form | Purity | bromine |
| | type¹⁾ | CISS¹⁾ | NaSS | No. | (area %) | (%) | (ppm) |
| Example 12 | NaSS | — | — | Example 1 | 0.01 | 99.5 | 108 |
| Example 13 | NaSS | — | — | Example 2 | ND | 99.5 | 46 |
| Example 14 | NaSS | — | — | Example 7 | 0.08 | 99.5 | 302 |
| Example 15 | ETSS | Example 15 | Example 12 | Example 1 | 0.01 | 94.0 | 84 |
| Example 16 | ETSS | Example 16 | Example 13 | Example 2 | ND | 95.0 | 51 |
| Example 17 | ETSS | Example 17 | Example 14 | Example 7 | 0.08 | 95.0 | 338 |
| Example 18 | NPSS | Example 15 | Example 12 | Example 1 | 0.01 | 97.5 | 122 |
| Example 19 | TfNS-Na | Example 15 | Example 12 | Example 1 | 0.01 | 99.5 | 101 |
| Example 20 | LiSS | — | Example 12 | Example 4 | 0.05 | 98.7 | 296 |
| Example 21 | NaSS | — | Example 12 | Example 1 | 0.01 | 99.5 | 108 |
| Example 22 | NaSS | — | Example 12 | Example 1 | 0.01 | 99.5 | 108 |
| Example 23 | BVBSl-Li | Example 15 | Example 12 | Example 1 | 0.01 | 94.0 | 121 |
| Synthesis | Crosslinked | (Example 20, 23) | | Example4, 1 | 0.05, 0.01 | — | — |
| Example 2 | product | | | | | | |

| | | | Bromide ion concentration in PSS | | | |
|---|---|---|---|---|---|---|
| | Chemical treatment after | | aqueous solution (ppm) | | | |
| | polymerization | | | Immediately | 70° | 70° |
| | | Heating | | after | C. × | C. × |
| | Additive²⁾ | condition | Mn | preparation | 7 days | 20 days |
| Example 12 | NaOH | 60° C. × 24 h | 114,000 | <1 | 4 | 16 |
| Example 13 | NaOH | 60° C. × 24 h | 112,000 | <1 | 3 | 10 |
| Example 14 | NaOH | 60° C. × 24 h | 113,000 | <1 | 12 | 26 |
| Example 15 | NaOH | 100° C. × 5 h | 9,000 | <1 | 2 | 15 |
| Example 16 | NaOH | 100° C. × 5 h | 9,000 | <1 | 2 | 9 |
| Example 17 | NaOH | 100° C. × 5 h | 9,000 | <1 | 13 | 24 |
| Example 18 | TMS-I | room | 18,000 | <1 | 3 | 15 |
| | | temperature × 4 h | | | | |
| Example 19 | — | — | 35000⁵⁾ | <1 | 3 | 16 |
| Example 20 | NaOH | 60° C. × 24 h | 65,000 | <1 | 10 | 23 |
| Example 21 | — | — | 33,000³⁾ | <1 | 3 | 13 |
| Example 22 | — | — | 46,000⁴⁾ | <1 | 3 | 12 |
| Example 23 | — | — | — | — | — | — |
| Synthesis | — | — | — | — | — | — |
| Example 2 | | | | | | |

¹⁾CISS = 4-styrenesulfonyl chloride
NaSS = sodium styrenesulfonate
LiSS = 4-lithium styrenesulfonate
ETSS = ethyl 4-styrenesulfonic
NPSS = neopentyl 4-styrenesulfonate
TfNS-Na = 4-styrenesulfonyl(trifluoromethylsulfonylimide) sodium
P-TfNS = poly[4-styrenesulfonyl(trifluoromethylsulfonylimide)]
BVBSl-Li = lithium bis-(4-styrenesulfonyl)imide
Crosslinked product = LiSS/BVBSI-Li copolymer
PSS = poly(4-styrenesulfonic acid) (10 wt % aqueous solution)
St = styrene
MAA = methacrylic acid
²⁾TMS-I = trimethylsilyl iodide
P = sodium hypophosphite
Pd-C = palladium carbon (Pd5%)
³⁾NaSS/St copolymer
⁴⁾NaSS/MAA copolymer
⁵⁾P-TfNS

### Comparative Example 8: Production of Sodium 4-Styrenesulfonate (NaSS) (4)

### <Production of NaSS>

Except that the 69.7 wt % BEBS aqueous solution obtained in Comparative Example 5 was used, exactly the same procedure as in Example 12 was carried out to obtain 311.60 g of a NaSS wet cake.

### <Purification of NaSS>

Except that the NaSS wet cake obtained as described above was used, exactly the same procedure as in Example 12 was carried out to obtain 273.70 g of a wet cake of purified NaSS.

Ion-exchange treatment was performed on the purified NaSS by the same procedure as in Example 12 to obtain 32.80 g of dry crystals of high-purity NaSS. The purity was 99.3 wt %, the moisture content was at 0.7 wt %, and organic impurities such as isomers which were analyzed by high-performance chromatography (HPLC) were at 0.00% for (a) sodium ortho-styrenesulfonate, at 0.00% for (b) sodium 4-(2-bromoethyl)benzenesulfonate, at 0.32% for (c) sodium meta-styrenesulfonate, at 0.01% for (d) sodium bromostyrenesulfonate, and at 0.00% for (e) sodium 4-(2-hydroxyethyl)benzenesulfonate (where each value is an area ratio when the sum of HPLC peak areas of the organic impurities and NaSS is defined as 100). That is, the content of sodium bromostyrenesulfonate in the high-purity NaSS was the same as in Example 12. However, the total bromine content, that is, bonded bromine, which was quantified by combustion decomposition ion chromatography, was 413 ppm, a much larger value as compared to that in Examples 12 to 14 (Table 5). This may be because BEBS having a high content of nuclear-brominated BEBS was used as a raw material.

Thereafter, as in Examples, the high-purity NaSS was polymerized to form PSS, and a change in bromide ion concentration with time (the presence of labile bonded bromine) was examined.

### <Production of Poly NaSS>

The high-purity NaSS obtained as described above was polymerized under the same conditions as in Example 12 to obtain a poly NaSS aqueous solution having a number average molecular weight Mn of 112,000 and a weight average molecular weight Mw of 285,000.

### <Preparation of PSS and Confirmation of Stability>

The poly NaSS aqueous solution obtained as described above was purified under the same conditions as in Example 12 to obtain 235.97 g of a 10.00 wt % PSS aqueous solution. The number average molecular weight was 112,000, the weight average molecular weight was 285,000 (Mw/Mn=2.54), the bromide ion concentration was less than 1 ppm, and the sodium content was at less than 1 ppm.

The PSS aqueous solution obtained as described above was aged, and a change in bromide ion concentration was monitored. The results are shown in Table 5, which reveals that an increase in bromide ions with time is marked as compared to Examples 12 to 14. This may be because the amount of bonded bromine contained in NaSS is large, that is, the amount of nuclear-brominated forms contained in BEBS as a precursor is large.

### Comparative Example 9: Synthesis of Sodium 4-Styrenesulfonate (5)

### <Synthesis of NaSS>

Under exactly the same conditions as in Example 12 except that the 72.1 wt % BEBS aqueous solution obtained in Comparative Example 1 was used as a raw material, 325.46 g of wet crystals of NaSS were obtained.

### <Purification of NaSS>

Ion-exchange treatment was performed on the obtained NaSS under the same conditions as in Example 12 to obtain 32.50 g of crystals of high-purity NaSS. After drying, the purity was 99.5 wt %, the moisture content was at 0.5 wt %, bromide ions were at less than 1 ppm, and the total bromine content was at 665 ppm. It is evident that the total bromine amount is larger as compared to that in Examples 12 to 14 (Table 5). This may be because BEBS having a high content of nuclear-brominated BEBS was used as a raw material.

As in Example 12, the high-purity NaSS was polymerized to form PSS, and a change in bromide ion concentration with time (the presence of labile bonded bromine) was examined.

### <Synthesis of Poly NaSS>

Under exactly the same conditions as in Example 12 except that the high-purity NaSS obtained as described above was used, a poly NaSS aqueous solution was obtained. The number average molecular weight Mn was 113,000 and the weight average molecular weight Mw was 291,000 (Mw/Mn=2.56).

### <Preparation of PSS and Confirmation of Stability>

Under exactly the same conditions as in Example 12 except that the poly NaSS aqueous solution obtained as described above was used, ultrafiltration and ion-exchange treatment were performed to obtain 238.48 g of a 10.0 wt % PSS aqueous solution. The number average molecular weight was 113,000, the weight average molecular weight was 291,000, the bromide ion concentration was less than 1 ppm, and the sodium content was at less than 1 ppm.

The PSS aqueous solution obtained as described above was aged, and a change in bromide ion concentration was monitored. The results are shown in Table 5, which reveals that an increase in bromide ions with time is marked as compared to Examples 12 to 14. This may be because the amount of bonded bromine in NaSS is large, that is, the amount of nuclear-brominated forms contained in BEBS as a precursor is large.

### Comparative Example 10: Synthesis of Sodium 4-Styrenesulfonate (6)

### <Synthesis of NaSS>

Under exactly the same conditions as in Example 12 except that the 70.8 wt % BEBS aqueous solution obtained in Comparative Example 2 was used as a raw material, 315.02 g of wet crystals of NaSS were obtained.

### <Purification of NaSS>

Ion-exchange treatment was performed on the obtained NaSS under the same conditions as in Example 12 to obtain 32.00 g of crystals of high-purity NaSS. After drying, the purity was 99.5 wt %, the moisture content was at 0.5 wt %, bromide ions were at less than 1 ppm, and the total bromine content was at 1,264 ppm. It is evident that the total bromine amount is larger as compared to that in Examples 12 to 14 (Table 5). This may be because BEBS having a high content of nuclear-brominated BEBS was used as a raw material.

As in Example 12, the NaSS was polymerized to form PSS, and a change in bromide ion concentration with time (the presence of labile bonded bromine) was examined.

### <Synthesis of Poly NaSS>

Under exactly the same conditions as in Example 12 except that the high-purity NaSS obtained as described above was used, a poly NaSS aqueous solution was obtained. The number average molecular weight Mn was 112,000 and the weight average molecular weight Mw was 283,000 (Mw/Mn=2.53).

### <Preparation of PSS and Confirmation of Stability>

Under exactly the same conditions as in Example 12 except that the poly NaSS aqueous solution obtained as described above was used, ultrafiltration and ion-exchange treatment were performed to obtain 238.48 g of a 10.0 wt % PSS aqueous solution. The number average molecular weight was 112,000, the weight average molecular weight was 283,000 (Mw/Mn=2.53), the bromide ion concentration was less than 1 ppm, and the sodium content was at less than 1 ppm.

As in Example 12, the PSS aqueous solution was aged, and a change in bromide ion concentration was monitored. The results are shown in Table 5, which reveals that an increase in bromide ions with time is marked as compared to Examples 12 to 14. This may be because the amount of bonded bromine in NaSS is large, that is, the amount of nuclear-brominated forms contained in BEBS as a precursor is large.

### Comparative Example 11: Synthesis of Sodium 4-Styrenesulfonate (7)

### <Synthesis of NaSS>

Under exactly the same conditions as in Example 12 except that the 72.3 wt % BEBS aqueous solution obtained in Comparative Example 4 was used as a raw material, 327.31 g of wet crystals of NaSS were obtained.

### <Purification of NaSS>

Ion-exchange treatment was performed on the obtained NaSS under the same conditions as in Example 12 to obtain 31.60 g of crystals of high-purity NaSS. After drying, the purity was 99.4 wt %, the moisture content was at 0.6 wt %, bromide ions were at less than 1 ppm, and the total bromine content was at 4,463 ppm. It is evident that the total bromine amount is larger as compared to that in Examples 12 to 14. This may be because BEBS having a high content of nuclear-brominated BEBS was used as a raw material.

As in Example 12, the NaSS was polymerized to form PSS, and a change in bromide ion concentration with time (the presence of labile bonded bromine) was examined.

### <Synthesis of Poly NaSS>

Under exactly the same conditions as in Example 12 except that the high-purity NaSS obtained as described above was used, a poly NaSS aqueous solution was obtained. The number average molecular weight Mn was 113,000 and the weight average molecular weight Mw was 285,000 (Mw/Mn=2.52).

### <Preparation of PSS and Confirmation of Stability>

Under exactly the same conditions as in Example 12 except that the poly NaSS aqueous solution obtained as described above was used, ultrafiltration and ion-exchange treatment were performed to obtain 244.69 g of a 10.0 wt % PSS aqueous solution. The number average molecular weight was 113,000, the weight average molecular weight was 285,000 (Mw/Mn=2.52), the bromide ion concentration was less than 1 ppm, and the sodium content was at less than 1 ppm.

As in Example 12, the PSS aqueous solution was aged, and a change in bromide ion concentration was monitored. The results are shown in Table 5, which reveals that an increase in bromide ions with time is marked as compared to Examples 12 to 14. This may be because the amount of bonded bromine in NaSS is large, that is, the amount of nuclear-brominated forms contained in BEBS as a precursor is large.

### Comparative Example 12: Synthesis of Ethyl 4-Styrenesulfonate (4)

### <Synthesis of Ethyl 4-Styrenesulfonate>

By exactly the same procedure as in Example 15 except that the high-purity NaSS obtained in Comparative Example 9 was used as a raw material, 11.80 g of ETSS was obtained. The purity on an area % basis which was determined by gas chromatography was 93.0%. The bromine content in the ETSS which was quantified by ion chromatography, that is, the inorganic bromine content extracted with pure water, is at less than 1 ppm, and the total bromine content quantified by combustion decomposition ion chromatography is at 651 ppm. It is thus evident that the amount of a bromine content is larger as compared to that in Examples 15 to 17 (Table 5). This may be because NaSS derived from BEBS having a high content of nuclear-brominated BEBS was used as a raw material.

Further, by the following method, the ETSS was polymerized to form PSS, and a change in bromide ion concentration with time (the presence of labile bonded bromine) was examined.

### <Synthesis of Poly ETSS>

Under exactly the same conditions as in Example 15 except that the ETSS obtained as described above was used as a raw material, ETSS was polymerized to obtain poly ETSS.

### <Preparation of PSS and Confirmation of Stability>

Under exactly the same conditions as in Example 15 except that the poly ETSS obtained as described above was used as a raw material, 26.88 g of a 10.0 wt % PSS aqueous solution was obtained. The number average molecular weight was 9,000, the weight average molecular weight was 12,000 (Mw/Mn=1.33), the bromide ion concentration was less than 1 ppm, and the sodium content was at less than 1 ppm.

As in Example 12, the PSS aqueous solution was aged, and a change in bromide ion concentration was monitored. The results are shown in Table 5, which reveals that an increase in bromide ions with time is marked as compared to Examples 15 to 17. This may be because the amount of bonded bromine in ETSS is large, that is, the amount of nuclear-brominated forms contained in BEBS as a precursor is large.

### Comparative Example 13: Synthesis of Ethyl 4-Styrenesulfonate (5)

### <Synthesis of ETSS>

Under exactly the same conditions as in Example 15 except that the high-purity NaSS obtained in Comparative Example 10 was used as a raw material, 11.90 g of ETSS was obtained. The purity on an area % basis which was determined by gas chromatography was 93.0%. The bromine content in the ETSS which was quantified by ion chromatography, that is, the inorganic bromine content extracted with pure water, is at less than 1 ppm, and the total bromine content quantified by combustion decomposition ion chromatography is at 1,331 ppm. It is thus evident that the amount of a bromine content is larger as compared to that in Examples 15 to 17 (Table 5). This may be because NaSS derived from BEBS having a high content of nuclear-brominated BEBS was used as a raw material.

Further, by the following method, the ETSS was polymerized to form PSS, and a change in bromide ion concentration with time (the presence of labile bonded bromine) was examined.

### <Synthesis of Poly ETSS>

Under exactly the same conditions as in Example 15 except that the ETSS obtained as described above was used as a raw material, ETSS was polymerized to obtain poly ETSS.

### <Preparation of PSS and Confirmation of Stability>

By exactly the same procedure as in Example 15 except that the poly ETSS obtained as described above was used as a raw material, 26.99 g of a 10.0 wt % PSS aqueous solution was obtained. The number average molecular weight was 9,000, the weight average molecular weight was 12,000 (Mw/Mn=1.33), the bromide ion concentration was less than 1 ppm, and the sodium content was at less than 1 ppm.

As in Example 12, the PSS aqueous solution was aged, and a change in bromide ion concentration was monitored. The results are shown in Table 5, which reveals that an increase in bromide ions with time is marked as compared to Examples 15 to 17. This may be because the amount of bonded bromine in ETSS is large, that is, the amount of nuclear-brominated forms contained in BEBS as a precursor is large.

### Comparative Example 14: Synthesis of Ethyl 4-Styrenesulfonate (6)

### <Synthesis of ETSS>

Under exactly the same conditions as in Example 15 except that the high-purity NaSS obtained in Comparative Example 11 was used as a raw material, 11.95 g of ETSS was obtained. The purity on an area % basis which was determined by gas chromatography was 94.0%. The bromine content in the ETSS which was quantified by ion chromatography, that is, the inorganic bromine content extracted with pure water, is at less than 1 ppm, and the total bromine content quantified by combustion decomposition ion chromatography is at 4,667 ppm. It is thus evident that the amount of a bromine content is larger as compared to that in Examples 15 to 17 (Table 5). This may be because NaSS derived from BEBS having a high content of nuclear-brominated BEBS was used as a raw material.

Further, by the following method, the ETSS was polymerized to form PSS, and a change in bromide ion concentration with time (the presence of labile bonded bromine) was examined.

<Synthesis of poly ETSS>

Under exactly the same conditions as in Example 15 except that the ETSS obtained as described above was used as a raw material, ETSS was polymerized to obtain poly ETSS.

### <Preparation of PSS and Confirmation of Stability>

By exactly the same procedure as in Example 15 except that the poly ETSS obtained as described above was used as a raw material, 26.10 g of a 10.0 wt % PSS aqueous solution was obtained. The number average molecular weight was 9,000, the weight average molecular weight was 11,000 (Mw/Mn=1.22), the bromide ion concentration was less than 1 ppm, and the sodium content was at less than 1 ppm.

The PSS aqueous solution was aged, and a change in bromide ion concentration was monitored. The results are shown in Table 5, which reveals that an increase in bromide ions with time is marked as compared to Examples 15 to 17. This may be because the amount of bonded bromine in ETSS is large, that is, the amount of nuclear-brominated forms contained in BEBS as a precursor is large.

### Comparative Example 15: Synthesis of Neopentyl 4-Styrenesulfonate (2)

### <Synthesis of NPSS>

A CISS solution was prepared under exactly the same conditions of charge weight and the like as in Example 15 except that the high-purity NaSS obtained in Comparative Example 9 was used as a raw material, and 35.10 g of white crystals of NPSS were obtained under the same conditions as in Example 18. The yield with respect to CISS was 24%, and the purity determined by ¹H-NMR (internal standard substance: 1,3,5-trimethylbenzene) was 97.3%. The bromine content in the NPSS which was quantified by ion chromatography, that is, the inorganic bromine content extracted with pure water, is at less than 1 ppm, and the total bromine content quantified by combustion decomposition ion chromatography is at 649 ppm. It is thus evident that the amount of a bromine content is larger as compared to that in Example 18 (Table 5). This may be because NaSS derived from BEBS having a high content of nuclear-brominated BEBS was used as a raw material.

As in Example 18, the NPSS was polymerized to form PSS, and a change in bromide ion concentration with time (the presence of labile bonded bromine) was examined.

### <Synthesis of Poly NPSS>

By exactly the same procedure as in Example 18 except that the NPSS obtained as described above was used as a raw material, 6.99 g of poly NPSS was obtained (the yield with respect to NPSS is 71%).

### <Preparation of PSS and Confirmation of Stability>

Except that poly NPSS obtained as described above was used as a raw material, exactly the same procedure as in Example 18 was carried out to obtain 57.32 g of a 10.00 wt % PSS aqueous solution. The number average molecular weight was 9,000, the weight average molecular weight was 11,000 (Mw/Mn=1.22), the bromide ion concentration was less than 1 ppm, and the sodium content was at less than 1 ppm.

As in Example 12, the PSS aqueous solution was aged, and a change in bromide ion concentration was monitored. The results are shown in Table 5, which reveals that an increase in bromide ions with time is marked as compared to Example 18. This may be because the amount of bonded bromine in NPSS is large, that is, the amount of nuclear-brominated forms which may be contained in BEBS as a precursor is large.

### Comparative Example 16: Production of 4-styrenesulfonyl(trifluoromethylsulfonylimide) sodium (TfNS-Na) (2)

### <Synthesis of TfNS-Na>

By exactly the same procedure as in Example 19 except that the high-purity NaSS obtained in Comparative Example 9 was used as a raw material, 24.65 g of TfNS-Na was obtained (yield 73%).

### <Purification of TfNS-Na>

Ion-exchange was performed on the obtained TfNS-Na by the same procedure as in Example 19, followed by neutralization with sodium hydroxide, thereby obtaining 20.60 g of crystals of high-purity TfNS-Na. The purity after drying which was determined by ¹H-NMR (internal standard substance: 1,3,5-trimethylbenzene) was 98.3 wt %, the moisture content was at 1.5 wt %, the bromide ion concentration determined by ion chromatography was less than 1 ppm, and the total bromine content determined by combustion decomposition ion chromatography was at 642 ppm. It is evident that the amount of bonded bromine is larger as compared to that in Example 19 (Table 5). This may be because NaSS derived from BEBS having a high content of nuclear-brominated BEBS was used as a raw material.

As in Example 19, poly TfNS-H was formed from TfNS-Na, and a change in bromide ion concentration with time (the presence of labile bonded bromine) was examined.

### <Preparation of Poly TfNS-H and Confirmation of Stability>

Under exactly the same conditions as in Example 19 except that the TfNS-Na obtained as described above was used, poly TfNS-Na was synthesized. The polymerization conversion rate was 98.7%, the number average molecular weight was 35,000, and the weight average molecular weight was 82,000 (Mw/Mn=2.34). Subsequently, ultrafiltration and ion-exchange treatment were performed under the same conditions as in Example 19 to obtain 149.79 g of a 10 wt % poly TfNS-H aqueous solution. The bromide ion concentration was less than 1 ppm, and the sodium content was at less 1 ppm.

As in Example 12, the poly TfNS aqueous solution was aged, and a change in bromide ion concentration was monitored. The results are shown in Table 5, which reveals that an increase in bromide ions with time is marked as compared to Example 19. This may be because the amount of bonded bromine in TINS-Na is large, that is, the amount of nuclear-brominated forms which may be contained in BEBS as a precursor is large.

### Comparative Example 17: Production of Lithium 4-Styrenesulfonate (2)

### <Synthesis of LiSS>

Under exactly the same conditions as in Example 20 except that the 72.3 wt % BEBS aqueous solution of Comparative Example 4 was used as a raw material, 203.50 g of a wet cake of LiSS was obtained.

### <Purification of LiSS>

The LiSS was purified under the same conditions as in Example 20 to obtain 73.50 g of dry LiSS.

The purity was 98.6 wt %, the moisture content was at 1.40 wt %, bromide ions were at less than 1 ppm, and the total bromine content was at 4,339 ppm. It is evident that the total bromine amount is larger as compared to that in Example 20 (Table 5). This may be because the amount of nuclear-brominated BEBS in BEBS used as a raw material was large.

As in Example 20, LiSS was polymerized to form PSS, and a change in bromide ion concentration with time (the presence of labile bonded bromine) was examined.

### <Synthesis of Poly LiSS>

Polymerization was performed under exactly the same conditions as in Example 20 except that the LiSS obtained as described above was used. The polymerization conversion rate was 99.7%, the number average molecular weight was 39,000, and the weight average molecular weight was 91,000 (Mw/Mn=2.33).

### <Preparation of PSS and Confirmation of Stability>

Ultrafiltration and ion-exchange treatment were performed on the obtained poly LiSS aqueous solution under the same conditions as in Example 20 to obtain 242.56 g of a 10.0 wt % PSS aqueous solution. The bromide ion concentration was less than 1 ppm, and the sodium content was at less than 1 ppm.

As in Example 20, the PSS aqueous solution obtained as described above was aged, and a change in bromide ion concentration was monitored. The results are shown in Table 5, which reveals that an increase in bromide ions with time is larger as compared to Example 20. This may be because the amount of bonded bromine in LiSS is large, that is, the amount of nuclear-brominated forms which may be contained in BEBS as a precursor is large.

### Comparative Example 18: Production of lithium bis-(4-styrenesulfonyl)imide (BVBSI-Li)

<Synthesis of 4-vinylbenzenesulfonamide>

Under exactly the same conditions of charge weight and the like as in Example 23 except that the CISS solution synthesized in Comparative Example 14 was used as a raw material, 6.10 g of a white solid of 4-styrenesulfonamide was obtained (yield 66%).

### <Synthesis of BVBSI-Li>

Under exactly the same conditions of charge weight and the like as in Example 23 except that the 4-styrenesulfonamide obtained as described above and the CISS solution synthesized in Comparative Example 14 were used, 6.25 g of white crystals of BVBSI-Li were obtained. The yield with respect to CISS was 60%, and the purity determined by ¹H-NMR (internal standard substance: 1,3,5-trimethylbenzene) was 93.3%. The bromine content in the high-purity BVBSI-Li which was quantified by ion chromatography, that is, the inorganic bromine content analyzed in the form of an aqueous solution, is at less than 1 ppm, and the total bromine content quantified by combustion decomposition ion chromatography is at 4,556 ppm.

### Example 24: Production of polystyrenesulfonic acid (PSS) (1)

### <Synthesis of Poly NaSS>

Except that after high-purity NaSS was polymerized, 1.65 g of a 48 wt % sodium hydroxide aqueous solution and 1.86 g of sodium hypophosphite monohydrate were added, and the mixture was continuously stirred at 110° C. for 15 hours while the solution was maintained at a pH of 13 or more, instead of adding 1.64 g of a 48 wt % sodium hydroxide aqueous solution and heating the mixture at 60° C. for 24 hours, under nitrogen flow, the same procedure as in Example 12 was carried out to obtain a poly NaSS aqueous solution.

The poly NaSS had a number average molecular weight Mn of 114,000 and a weight average molecular weight Mw of 285,000 (Mw/Mn=2.50).

### <Preparation of PSS and Confirmation of Stability>

The poly NaSS aqueous solution was purified by the same procedure as in Example 12 to obtain 230.05 g of a 10.00 wt % PSS aqueous solution. The number average molecular weight was 114,000, the weight average molecular weight was 282,000 (Mw/Mn=2.47), the bromide ion concentration was less than 1 ppm, and the sodium content was at less than 1 ppm. As in Example 12, solid PSS was acquired, and the halogen content was analyzed. The results showed that the total bromine content was at 63 ppm, a value smaller than that in Example 12. This may be because a part of bonded bromine was released by appropriately performing chemical treatment before purification of the poly NaSS. Note that the total chlorine content in the solid PSS was at less than 1 ppm.

As in Example 12, the 10 wt % PSS aqueous solution was aged at 70° C., and a change in bromide ion concentration was monitored. The results are shown in Table 5, which reveals that an increase in bromide ions with time is further suppressed as compared to Example 12.

### Example 25: Production of polystyrenesulfonic acid (PSS) (2)

### <Synthesis of Poly NaSS>

Except that after high-purity NaSS was polymerized, 1.65 g of a 48 wt % sodium hydroxide aqueous solution was added, and the mixture was continuously stirred at 110° C. for 20 hours while the solution was maintained at a pH of 13 or more, instead of adding 1.64 g of a 48 wt % sodium hydroxide aqueous solution and heating the mixture at 60° C. for 24 hours, under nitrogen flow, the same procedure as in Example 13 was carried out to obtain a poly NaSS aqueous solution.

The poly NaSS had a number average molecular weight Mn of 114,000 and a weight average molecular weight Mw of 285,000 (Mw/Mn=2.50).

### <Preparation of PSS and Confirmation of Stability>

The poly NaSS aqueous solution obtained as described above was purified by the same procedure as in Example 13 to obtain 231.30 g of a 10.00 wt % PSS aqueous solution. In the PSS, the number average molecular weight was 112,000, the weight average molecular weight was 281,000 (Mw/Mn=2.51), the bromide ion concentration was less than 1 ppm, and the sodium content was at less than 1 ppm. As in Example 12, solid PSS was acquired, and the total bromine content was analyzed. The result showed that the total bromine content was at 35 ppm, a value smaller than that in Example 13. This may be because a part of bonded bromine was released by appropriately performing chemical treatment before purification of the poly NaSS. Note that the total chlorine content in the solid PSS was at less than 1 ppm.

As in Example 13, the 10 wt % PSS aqueous solution was aged at 70° C., and a change in bromide ion concentration was monitored. The results are shown in Table 5, which reveals that an increase in bromide ions with time is further suppressed as compared to Example 13.

### Example 26: Production of polystyrenesulfonic acid (PSS) (3)

### <Synthesis of Poly NaSS>

Except that after high-purity NaSS was polymerized, 2.01 g of a 48 wt % sodium hydroxide aqueous solution and 1.90 g of sodium hypophosphite monohydrate were added, and the mixture was continuously stirred at 110° C. for 15 hours while the solution was maintained at a pH of 13 or more, instead of adding 1.64 g of a 48 wt % sodium hydroxide aqueous solution and heating the mixture at 60° C. for 24 hours, under nitrogen flow, the same procedure as in Example 14 was carried out to obtain a poly NaSS aqueous solution.

The poly NaSS had a number average molecular weight Mn of 114,000 and a weight average molecular weight Mw of 285,000 (Mw/Mn=2.50).

### <Preparation of PSS and Confirmation of Stability>

The poly NaSS aqueous solution obtained as described above was purified by the same procedure as in Example 14 to obtain 232.02 g of a 10.00 wt % PSS aqueous solution. In the PSS, the number average molecular weight was 113,000, the weight average molecular weight was 282,000 (Mw/Mn=2.50), the bromide ion concentration was less than 1 ppm, and the sodium content was at less than 1 ppm. As in Example 12, solid PSS was acquired, and the total bromine content was analyzed. The result showed that the total bromine content was at 91 ppm, a value smaller than that in Example 14. This may be because a part of bonded bromine was released by appropriately performing chemical treatment before purification of the poly NaSS. Note that the total chlorine content in the solid PSS was at less than 1 ppm.

As in Example 14, the 10 wt % PSS aqueous solution was aged at 70° C., and a change in bromide ion concentration was monitored. The results are shown in Table 5, which reveals that an increase in bromide ions with time is further suppressed as compared to Example 14. This may be because a part of bonded bromine was released by appropriately performing chemical treatment before purification of the poly NaSS.

### Example 27: Production of Styrenesulfonic Acid/Styrene (SS/St) Copolymer

### <Synthesis of NaSS/Styrene Copolymer>

Except that 1.65 g of a 48 wt % sodium hydroxide aqueous solution was added to the 15 wt % NaSS/styrene copolymer aqueous solution before purification and the mixture was stirred at 90° C. for 24 hours while the solution was maintained at a pH of 13 or more, under nitrogen flow, the same procedure as in Example 21 was carried out to obtain a NaSS/styrene copolymer aqueous solution.

The copolymer had a number average molecular weight Mn of 33,000 and a weight average molecular weight Mw of 74,000 (Mw/Mn=2.24).

### <Preparation of PSS and Confirmation of Stability>

The NaSS/styrene copolymer obtained as described above was purified by the same procedure as in Example 21 to obtain 229.50 g of a 10.00 wt % styrenesulfonic acid/styrene copolymer aqueous solution. In the acid-type copolymer, the number average molecular weight Mn was 33,000, the weigh average molecular weight Mw was 74,000 (Mw/Mn=2.24), the bromide ion concentration was less than 1 ppm, and the sodium content was at less than 1 ppm.

The 10 wt % copolymer aqueous solution was aged at 70° C., and a change in bromide ion concentration was monitored. The results are shown in Table 5, which reveals that an increase in bromide ions with time is further suppressed as compared to Example 21. This may be because a part of bonded bromine was released by appropriately performing chemical treatment before purification of the polymer.

### Example 28: Production of Styrenesulfonic Acid/Methacrylic Acid (SS/MAA) Copolymer

### <Synthesis of NaSS/Methacrylic Acid Copolymer>

Except that 5.00 g of a 48 wt % sodium hydroxide aqueous solution was added to the 15 wt % NaSS/MAA copolymer aqueous solution before purification and the mixture was continuously stirred at 90° C. for 24 hours while the solution was maintained at a pH of 13 or more, under nitrogen flow, the same procedure as in Example 22 was carried out to obtain a NaSS/methacrylic acid copolymer. The number average molecular weight was 46,000, the weigh average molecular weight was 129,000 (Mw/Mn=2.80).

### <Synthesis of Styrenesulfonic Acid/Methacrylic Acid Copolymer and Confirmation of Stability>

Ultrafiltration and ion-exchange treatment were performed on the NaSS/methacrylic acid copolymer under the same conditions as in Example 22 to obtain 258.06 g of a 10.0 wt % aqueous solution of a styrenesulfonic acid/methacrylic acid copolymer. The bromide ion concentration was less than 1 ppm, and the sodium content was at less than 1 ppm. As in Example 12, a solid form of the styrenesulfonic acid/methacrylic acid copolymer was acquired, and the total bromine content was analyzed. The result showed that the total bromine content was at 45 ppm. The total chlorine content was at less than 1 ppm.

### <Stability of Styrenesulfonic Acid/Methacrylic Acid Copolymer>

As in Example 22, the 10 wt % styrenesulfonic acid/methacrylic acid copolymer aqueous solution was aged at 70° C., and a change in bromide ion concentration was monitored. The results are shown in Table 5, which reveals that an increase in bromide ions with time is further suppressed as compared to Example 22. This may be because a part of bonded bromine was released by appropriately performing chemical treatment before purification of the polymer.

### Example 29: Production of Polystyrenesulfonic Acid (PSS) (4)

### <Synthesis of Poly NaSS>

The same procedure as in Example 12 was carried out except that after high-purity NaSS was polymerized, 0.50 g of sodium formate and 0.05 g of palladium-on-carbon (Pd content: 5 wt %) were added and the mixture was continuously stirred at 90° C. for 24 hours instead of adding 1.64 g of a 48 wt % sodium hydroxide aqueous solution and heating the mixture at 60° C. for 24 hours, under nitrogen flow. Thereafter, the polymer solution was filtered through a membrane filter with a pore diameter of 0.45 μm to remove the palladium-on-carbon.

The poly NaSS had a number average molecular weight Mn of 114,000 and a weight average molecular weight Mw of 285,000 (Mw/Mn=2.50).

### <Preparation of PSS and Confirmation of Stability>

The poly NaSS aqueous solution obtained as described above was purified by the same procedure as in Example 12 to obtain 230.36 g of a 10.00 wt % PSS aqueous solution. In the PSS, the number average molecular weight was 114,000, the weight average molecular weight was 282,000, the bromide ion concentration was less than 1 ppm, and the sodium content was at less than 1 ppm. As in Example 12, a solid form of the copolymer was acquired, and the total bromine content was analyzed. The result showed that the total bromine content was at 51 ppm. The total chlorine content was at less than 1 ppm.

As in Example 12, the 10 wt % PSS aqueous solution was aged at 70° C., and a change in bromide ion concentration was monitored. The results are shown in Table 5, which reveals that an increase in bromide ions with time is further suppressed as compared to Example 14. This may be because a part of bonded bromine was released by chemical treatment of poly NaSS.

### Comparative Example 19: Production of Polystyrenesulfonic Acid

### <Synthesis of Poly NaSS>

Except that after high-purity NaSS was polymerized, 2.02 g of a 48 wt % sodium hydroxide aqueous solution was added, and the mixture was stirred at 110° C. for 15 hours while the solution was maintained at a pH of 13 or more, instead of adding 1.64 g of a 48 wt % sodium hydroxide aqueous solution and heating the mixture at 60° C. for 24 hours, under nitrogen flow, the same procedure as in Comparative Example 11 was carried out to obtain a poly NaSS aqueous solution. The number average molecular weight Mn was 113,000, the weigh average molecular weight Mw was 285,000 (Mw/Mn=2.52).

### <Preparation of PSS and Confirmation of Stability>

Under exactly the same conditions as in Comparative Example 11 except that the poly NaSS aqueous solution obtained as described above was used, ultrafiltration and ion-exchange treatment were performed to obtain 240.61 g of a 10.0 wt % PSS aqueous solution. The number average molecular weight was 113,000, the weight average molecular weight was 283,000, the bromide ion concentration was less than 1 ppm, and the sodium content was at less than 1 ppm. As in Example 12, solid PSS was acquired, and the total bromine content was analyzed. The result showed that the total bromine content was at 2,721 ppm. The total bromine content was at less than 1 ppm.

As in Comparative Example 11, the 10 wt % PSS aqueous solution was aged, and a change in bromide ion concentration was monitored. The results are shown in Table 5, which reveals that an increase in bromide ions with time is half that in Comparative Example 11, but is markedly larger as compared to that in Examples 12 to 14 and 25. This may be because the amount of bonded bromine in NaSS was too large to control, that is, the amount of nuclear-brominated forms contained in BEBS as a precursor was too large to control although poly NaSS was chemically treated under proper conditions.

TABLE 5
Table 5 Example of production of styrenesulfonic acid compound

| | | | | BEBS property | | Monomer property | |
|---|---|---|---|---|---|---|---|
| | | | | | Nuclear- | | Total |
| | | Raw | Raw | | brominated | | bonded |
| | Monomer | material | material | | form | Purity | bromine |
| | type¹⁾ | CISS¹⁾ | NaSS | No. | (area %) | (%) | (ppm) |
| Comparative | NaSS | — | | Comparative | 0.19 | 99.3 | 413 |
| Example 8 | | | | Example 5 | | | |
| Comparative | NaSS | — | | Comparative | 0.31 | 99.5 | 665 |
| Example 9 | | | | Example 1 | | | |
| Comparative | NaSS | — | | Comparative | 0.54 | 99.5 | 1,264 |
| Example 10 | | | | Example 2 | | | |
| Comparative | NaSS | — | | Comparative | 2.01 | 99.4 | 4,463 |
| Example 11 | | | | Example 4 | | | |
| Comparative | ETSS | Comparative | Comparative | Comparative | 0.31 | 93.0 | 651 |
| Example 12 | | Example 12 | Example 9 | Example 1 | | | |
| Comparative | ETSS | Comparative | Comparative | Comparative | 0.54 | 93.0 | 1,331 |
| Example 13 | | Example 13 | Example 10 | Example 2 | | | |
| Comparative | ETSS | Comparative | Comparative | Comparative | 2.01 | 94.0 | 4,667 |
| Example 14 | | Example 14 | Example 11 | Example 4 | | | |
| Comparative | NPSS | Comparative | Comparative | Comparative | 0.31 | 97.3 | 649 |
| Example 15 | | Example 12 | Example 9 | Example 1 | | | |
| Comparative | TfNS-Na | Comparative | Comparative | Comparative | 0.31 | 98.3 | 642 |
| Example 16 | | Example 12 | Example 9 | Example 1 | | | |
| Comparative | LISS | | | Comparative | 2.01 | 98.6 | 4,339 |
| Example 17 | | | | Example 4 | | | |
| Comparative | BVBSI-Li | Example 14 | Example 11 | Example 4 | 2.01 | 93.3 | 4,556 |
| Example 18 | | Comparative | Comparative | Comparative | | | |
| Example 24 | NaSS | — | Example 12 | Example 1 | 0.01 | 99.5 | 108 |
| Example 25 | NaSS | — | Example 13 | Example 2 | ND | 99.5 | 46 |
| Example 26 | NaSS | — | Example 14 | Example 7 | 0.08 | 99.5 | 302 |
| Example 27 | NaSS | — | Example 12 | Example 1 | 0.01 | 99.5 | 108 |
| Example 28 | NaSS | — | Example 12 | Example 1 | 0.01 | 99.5 | 108 |
| Example 29 | NaSS | — | Example 14 | Example 7 | 0.08 | 99.5 | 302 |
| Comparative | NaSS | — | Comparative | Comparative | 2.01 | 99.4 | 4,463 |
| Example 19 | | | Example 11 | Example 4 | | | |

| | | | Bromide ion concentration | | | |
|---|---|---|---|---|---|---|
| | Chemical treatment | | in PSS aqueous solution (ppm) | | | |
| | after polymerization | | | Immediately | | |
| | | Heating | | after | 70° C. × | 70° C. × |
| | Additive²⁾ | condition | Mn | preparation | 7 days | 20 days |
| Comparative | NaOH | 60° C. × | 112,000 | <1 | 21 | 55 |
| Example 8 | | 24 h | | | | |
| Comparative | NaOH | 60° C. × | 113,000 | <1 | 24 | 78 |
| Example 9 | | 24 h | | | | |
| Comparative | NaOH | 60° C. × | 112,000 | <1 | 36 | 101 |
| Example 10 | | 24 h | | | | |
| Comparative | NaOH | 60° C. × | 113,000 | <1 | 58 | 302 |
| Example 11 | | 24 h | | | | |
| Comparative | NaOH | 100° C. × | 9,000 | <1 | 23 | 67 |
| Example 12 | | 5 h | | | | |
| Comparative | NaOH | 100° C. × | 9,000 | <1 | 39 | 113 |
| Example 13 | | 5 h | | | | |
| Comparative | NaOH | 100° C. × | 9,000 | <1 | 60 | 334 |
| Example 14 | | 5 h | | | | |
| Comparative | TMS-I | room | 9,000 | <1 | 22 | 59 |
| Example 15 | | temper- | | | | |
| | | ature × | | | | |
| | | 4 h | | | | |
| Comparative | — | — | 35,000⁵⁾ | <1 | 23 | 61 |
| Example 16 | | | | | | |
| Comparative | NaOH | 60° C. × | 39,000 | <1 | 67 | 351 |
| Example 17 | | 24 h | | | | |
| Comparative | — | — | — | — | — | — |
| Example 18 | | | | | | |
| Example 24 | NaOH/P | 110° C. × | 114,000 | <1 | 1 | 3 |
| | | 15 h | | | | |
| Example 25 | NaOH | 110° C. × | 112,000 | <1 | 1 | 2 |
| | | 15 h | | | | |
| Example 26 | NaOH/P | 110° C. × | 113,000 | <1 | 3 | 6 |
| | | 15 h | | | | |
| Example 27 | NaOH | 90° C. × | 33,000³⁾ | <1 | 1 | 2 |
| | | 24 | | | | |
| Example 28 | NaOH | 90° C. × | 46,000⁴⁾ | <1 | 1 | 2 |
| | | 24 | | | | |
| Example 29 | formic | 90° C. × | 113,000 | <1 | 5 | 10 |
| | acid/Pd—C | 24 | | | | |
| Comparative | NaOH | 110° C. × | 113,000 | <1 | 34 | 163 |
| Example 19 | | 15 h | | | | |

¹⁾CISS = 4-styrenesulfonyl chloride
NaSS = 4-sodium styrenesulfonate
LiSS = 4-lithium styrenesulfonate
ETSS = 4-ethyl styrenesulfonate
NPSS = neopentyl 4-styrenesulfonate
TfNS-Na = 4-styrenesulfonyl(trifluoromethylsulfonylimide) sodium
P-TfNS = poly[4-styrenesulfonyl(trifluoromethylsulfonylimide)]
BVBSI-Li = lithium bis-(4-styrenesulfonyl)imide
Crosslinked product = LiSS/BVBSI-Li copolymer
PSS = poly(4-styrenesulfonic acid) (10 wt % aqueous solution)
St = styrene
MAA = methacrylic acid
²⁾TMS-I = trimethylsilyl iodide
P = sodium hypophosphite
Pd—C = palladium carbon (Pd5%)
³⁾NaSS/St copolymer
⁴⁾NaSS/MAA copolymer
⁵⁾P-TfNS

### Example 30: Production of Polystyrenesulfonic Acid Composition (1)

To the 10 wt % styrenesulfonic acid/styrene copolymer aqueous solution obtained in Example 27, hydroquinone was added (700 ppm with respect to the polymer net content). The mixture was divided among sample bottles, sealed, and aged in an oven at 70° C. to monitor changes in molecular weight and bromide ion concentration. The results are shown in Table 6, which reveals that an increase in bromide ions was small, and a decrease in molecular weight was markedly suppressed as compared to Comparative Example 20.

### Example 31: Production of Polystyrenesulfonic Acid Composition (2)

To the 10 wt % styrenesulfonic acid/styrene copolymer aqueous solution obtained in Example 27, 4-methoxyphenol was added (1500 ppm with respect to the polymer net content). As in Example 30, changes in weight average molecular weight and bromide ion concentration were monitored. The results are shown in Table 6, which reveals that an increase in bromide ions was small, and a decrease in molecular weight was markedly suppressed as compared to Comparative Example 20.

### Comparative Example 20: Production of Polystyrenesulfonic Acid Composition (3)

To the 10 wt % styrenesulfonic acid/styrene copolymer aqueous solution obtained in Example 27, 4-methoxyphenol was added (10 ppm with respect to the polymer net content). As in Example 30, changes in weight average molecular weight and bromide ion concentration were monitored. The results are shown in Table 6, which reveals that an increase in bromide ions is small, but a decrease in molecular weight is marked as compared to Examples 30 and 31.

TABLE 6

| | | | Comparative |
|---|---|---|---|
| | Example 30 | Example 31 | Example 20 |
| [Composition] | | | |
| 10 wt % styrenesulfonic | Example 27 | Example 27 | Example 27 |
| acid/styrene copolymer | | | |
| Phenolic antioxidant | HQ | MEHQ | MEHQ |
| addition amount(ppm) | 700 | 1,500 | 10 |
| [Stability] | | | |
| Weight average | | | |
| molecular weight Mw | | | |
| Immediately after preparation | 74,000 | 74,000 | 74,000 |
| After 300 days | 74,000 | 74,000 | 68,000 |
| at room temperature | | | |
| After 20 days at 70 [symbol] C. | 73,000 | 73,000 | 47,000 |
| Bromide ion | | | |
| concentration(ppm) | | | |
| Immediately after preparation | <1 | <1 | <1 |
| After 300 days | <1 | <1 | <1 |
| at room temperature | | | |
| After 20 days at 70 [symbol] C. | 3 | 3 | 4 |

HQ = hydroquinone
MEHQ = 4-methoxyphenol

### INDUSTRIAL APPLICABILITY

The 4-(2-bromoethyl)benzenesulfonic acid having a decreased amount of nuclear-brominated forms according to the present invention is useful as a precursor for use in production of a styrenesulfonic acid compound having a decreased amount of bonded bromine and a polymer thereof, and the styrenesulfonic acid compound having a decreased amount of bonded bromine and a polymer thereof are extremely useful for modifiers for secondary batteries, dopants for conductive polymers, additives for semiconductor polishing and cleaning agents, photoresists, and organic EL elements, and particularly for electronic materials.

### EXPLANATION OF REFERENCE SIGNS

A peak of 4-(2-hydroxyethyl)benzenesulfonic acid
B peak of para-isomer of BEBS
C peak of ortho-isomer of BEBS
D peak of 4-(1-bromoethyl)benzenesulfonic acid
E peak of 2-bromo-4-(2-bromoethyl)benzenesulfonic acid (nuclear-brominated BEBS)
a position of peak of sodium ortho-styrenesulfonate
b position of peak of sodium 4-(2-bromoethyl)benzenesulfonate
c position of peak of sodium meta-styrenesulfonate
d position of peak of sodium bromostyrenesulfonate
e position of peak derived from sodium 4-(2-hydroxyethyl)benzenesulfonate

1. A high-purity 4-(2-bromoethyl)benzenesulfonic acid composition in which a ratio of nuclear-brominated 2-bromoethylbenzenesulfonic acid represented by the following general formula (A) with respect to 4-(2-bromoethyl)benzenesulfonic acid is 0.10% or less [peak area % of nuclear-brominated 2-bromoethylbenzenesulfonic acid when the peak area of 4-(2-bromoethyl)benzenesulfonic acid is defined as 100% is determined by liquid chromatography (LC)]:

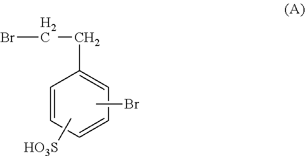

2. The high-purity 4-(2-bromoethyl)benzenesulfonic acid composition according to claim 1, wherein the nuclear-brominated 2-bromoethylbenzenesulfonic acid is 2-bromo-4-(2-bromoethyl)benzenesulfonic acid.

3. The high-purity 4-(2-bromoethyl)benzenesulfonic acid composition according to claim 1, wherein a purity of the 4-(2-bromoethyl)benzenesulfonic acid which is determined by liquid chromatography (LC) is 93 area % or more.

4. A method for producing the high-purity 4-(2-bromoethyl)benzenesulfonic acid composition according to claim 1, comprising continuously supplying 2-bromoethylbenzene or a solution of 2-bromoethylbenzene in an organic solvent and anhydrous sulfuric acid or a solution of anhydrous sulfuric acid in an organic solvent to a reactor, the method comprising carrying out the reaction while performing control so that the 2-bromoethylbenzene and the organic solvent each contain an iron content at 5 μg/g or less, hydrogen bromide at 100 ppm or less and a moisture content at 1,000 ppm or less, maintaining a weight percentage of the anhydrous sulfuric acid supplied at 5.00 wt % to 20.00 wt % with respect to the total reaction liquid inside the reactor, and maintaining a molar ratio of the anhydrous sulfuric acid at 0.50 to 2.00 with respect to the 2-bromoethylbenzene inside the reactor.

5. The production method according to claim 4, wherein the organic solvent is one or more organic solvents selected from the group consisting of a halogenated solvent, a nitrated solvent and an aliphatic hydrocarbon.

6. The production method according to claim 4, wherein the anhydrous sulfuric acid contains acetic acid or anhydrous acetic acid at 5 wt % to 10 wt % with respect to the anhydrous sulfuric acid.

7. The production method according to claim 4, wherein the anhydrous sulfuric acid or the solution of anhydrous sulfuric acid in an organic solvent is continuously supplied over 0.5 hours to 7 hours.

8. The production method according to claim 4, wherein in the reaction, a reaction temperature is 10 to 60° C. and a reaction time is 0.5 hours to 10 hours.

9. A method for producing the high-purity 4-(2-bromoethyl)benzenesulfonic acid composition according to claim 1, comprising continuously supplying anhydrous sulfuric acid or a solution of anhydrous sulfuric acid in an organic solvent to 2-bromoethylbenzene or a solution of 2-bromoethylbenzene in an organic solvent, the method comprising carrying out the reaction while performing control so that the 2-bromoethylbenzene and the organic solvent each contain an iron content at 5 g/g or less, hydrogen bromide at 100 ppm or less and a moisture content at 1,000 ppm or less, maintaining a weight percentage of the anhydrous sulfuric acid supplied at 20.00 wt % or less with respect to the total reaction liquid inside a reactor, and maintaining a molar ratio of the anhydrous sulfuric acid at 2.00 or less with respect to the 2-bromoethylbenzene inside the reactor.

10. A high-purity styrenesulfonic acid compound composition comprising a styrenesulfonic acid compound represented by the following general formula (B), wherein a bonded bromine content determined by combustion decomposition ion chromatography (CIC) is 400 ppm or less: 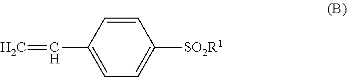
wherein R¹ represents the following general formula (C), the following general formula (D), an amino group, or a chlorine atom:
—OR² (C)
wherein R2 represents a substituted or unsubstituted alkyl group having 1 to 6 carbon atoms, a hydrogen atom, an alkali metal, a substituted or unsubstituted ammonium cation, or a substituted or unsubstituted phosphonium cation 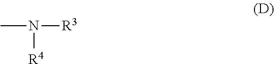
wherein R³ represents a substituted or unsubstituted alkyl group, a hydrogen atom, an alkali metal, or a substituted or unsubstituted ammonium cation, and R4 represents a trifluoromethylsulfonyl group, a perfluorobutylsulfonyl group, a fluorosulfonyl group, a trifluoromethylacetyl group, or a 4-ethenylphenylsulfonyl group.

11. A high-purity styrenesulfonic acid compound composition comprising a styrenesulfonic acid compound represented by the following general formula (B′), wherein the styrenesulfonic acid compound is sodium 4-styrenesulfonate, lithium 4-styrenesulfonate, potassium 4-styrenesulfonate, ammonium 4-styrenesulfonate, N,N-dimethylcyclohexylamine 4-styrenesulfonate, trioctylamine 4-styrenesulfonate, 4-styrenesulfonyl chloride, 4-styrenesulfonamide, ethyl 4-styrenesulfonate, neopentyl 4-styrenesulfonate, 4-styrenesulfonyl(trifluoromethylsulfonylimide), 4-styrenesulfonyl(perfluorobutylsulfonylimide), 4-styrenesulfonyl(fluorosulfonylimide) or lithium bis-(4-styrenesulfonyl)imide, and a bonded bromine content determined by combustion decomposition ion chromatography (CIC) is 400 ppm or less: 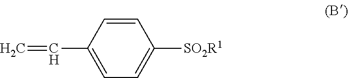
wherein R1 is the same as R1 in general formula (B) described in claim 10.

12. A polystyrenesulfonic acid compound composition having a decreased amount of bonded bromine, the polystyrenesulfonic acid compound composition comprising a polystyrenesulfonic acid compound having the following repeating structural unit (E), or a polystyrenesulfonic acid compound having the following repeating structural unit (E) and the following repeating structural unit (F), wherein a bromide ion concentration in a 10 wt % aqueous solution of the polystyrenesulfonic acid compound is 30 ppm or less when the aqueous solution is held at 70° C. for 20 days: 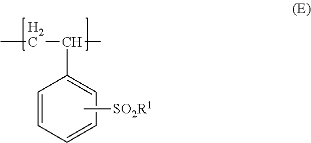
wherein R1 is the same as R1 in general formula (B) described in claim 10, 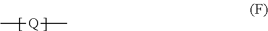
wherein Q represents a repeating structural unit derived from a vinyl monomer that can be copolymerized with a styrenesulfonic acid compound.

13. The polystyrenesulfonic acid compound composition according to claim 12, wherein a number average molecular weight of the polystyrenesulfonic acid compound is 500 to 5,000,000.

14. The polystyrenesulfonic acid compound composition according to claim 12, wherein Q in the repeating structural unit (F) comprises a repeating structural unit derived from a vinyl monomer which is one or a combination of two or more selected from the group consisting of (meth)acrylic acid, a (meth)acrylic acid ester, (meth)acrylamide, N-substituted maleimide, a styrene compound and vinyl pyridine.

15. The polystyrenesulfonic acid compound composition according to claim 12, wherein Q in the repeating structural unit (F) comprises a repeating structural unit derived from a crosslinkable monomer which is one or a combination of two or more selected from the group consisting of a substituted styrene compound, a (meth)acrylic acid ester compound, a (meth)acrylamide compound and a N-substituted maleimide compound.

16. The polystyrenesulfonic acid compound composition according to claim 12, wherein a bromide ion concentration in a 10 wt % aqueous solution of the polystyrenesulfonic acid compound is 10 ppm or less when the aqueous solution is held at 70° C. for 20 days.

17. A method for producing a polystyrenesulfonic acid compound, comprising chemically treating the polystyrenesulfonic acid compound composition according to claim 12 through the following step (i) or (ii):
(i) heating a solution of the polystyrenesulfonic acid compound composition at 90° C. to 110° C. for 5 hours to 30 hours while maintaining the solution at a pH of 13 or more by adding an alkali or an alkali and a reducing agent thereto, followed by purification of the polymer
(ii) adding a reducing agent and a palladium catalyst to a solution of the polystyrenesulfonic acid compound composition, and heating the mixture at 80° C. to 110° C. for 5 hours to 30 hours, followed by purification of the polymer.

18. A polystyrenesulfonic acid compound aqueous solution composition comprising the polystyrenesulfonic acid compound composition according to claim 12 and a phenolic antioxidant,
wherein a content of the phenolic antioxidant with respect to a net content of the polystyrenesulfonic acid compound is 20 ppm to 2,000 ppm.

19. The polystyrenesulfonic acid aqueous solution composition according to claim 18, wherein the phenolic antioxidant is at least one selected from the group consisting of 2-methoxyphenol, 3-methoxyphenol, 4-methoxyphenol, 2,6-di-tert-butylphenol, 2,4-di-tert-butylphenol, 2,6-di-tert-butyl-4-methylphenol, 4-tert-butylcatechol, hydroquinone and methoxyhydroquinone.
